SEQUENCE LISTING (38 sequences)

SEQ ID NO: 1 - protein, 7 aa, synthetic construct
QSGDLTR

SEQ ID NO: 2 - protein, 7 aa, synthetic construct
KHGNLSE

SEQ ID NO: 3 - protein, 7 aa, synthetic construct
KRCNLRC

SEQ ID NO: 4 - protein, 7 aa, synthetic construct
CPSHLTR

SEQ ID NO: 5 - protein, 7 aa, synthetic construct
RQFNRHQ

SEQ ID NO: 6 - DNA, 28 bp, synthetic construct
  source 1..28: mol_type = other DNA
agcagcagcagcagcagcagcagcagca

SEQ ID NO: 7 - protein, 7 aa, synthetic construct
SPEQLSR

SEQ ID NO: 8 - protein, 7 aa, synthetic construct
QWSTRKR

SEQ ID NO: 9 - protein, 7 aa, synthetic construct
KQGNLVE

SEQ ID NO: 10 - DNA, 828 bp, synthetic construct
  source 1..828: mol_type = other DNA
atggcccccaagaaaaagcggaaagtgggcatccacggggtacccgccgctatggctgagaggcccttccagtgtcgaatctgcatgcgtaacttcagttgtccgtcccacctgacccgccacatccgcacccacaccggcgagaagccttttgcctgtgacatttgtgggaggaaatttgcccagtccggcgacctgacccgccataccaagatacacacgcctaatcctcatcgccgcactgatcccagccataagcccttccagtgtcgaatctgcatgcgtaacttcagtaagcacggcaacctgtccgagcacatccgcacccacaccggcgagaagccttttgcctgtgacatttgtgggaggaaatttgccaagcgctgtaacctgcgctgtcataccaagatacacacgggctcccaatcccccttccagtgtcgaatctgcatgcgtaagtttgcccgccagttcaaccgccaccagcataccaagatacacctgcgccaaaaagatgcggcccggggatccggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagtttaa

SEQ ID NO: 11 - DNA, 828 bp, synthetic construct
  source 1..828: mol_type = other DNA
atggcccctaagaagaagagaaaagttggcatacatggagtccctgctgcaatggctgaaagaccattccagtgcagaatctgcatgaggaacttctcctgcccttcccacctgaccaggcacatcaggacccacactggagaaaagccctttgcctgtgatatctgtggaagaaaatttgcccagtcaggggacctcaccagacacaccaaaattcacacccccaacccccatagaagaactgacccctcccataagcccttccagtgccggatctgcatgcggaacttcagcaagcatggcaacctgtcagagcacattaggactcacactggggagaagcccttcgcctgtgacatctgtggccgcaagtttgccaagaggtgcaacctgaggtgccataccaagatccacactggatcccagtccccattccaatgcagaatttgcatgagaaagtttgctagacaattcaacagacaccagcacaccaagattcacctgaggcagaaggatgcagccagaggttcaggaatggatgccaagtccctcactgcctggagcagaaccctggtcaccttcaaggatgtgtttgtggacttcaccagagaagagtggaagctcctggacactgcccagcaaattgtgtacaggaatgtgatgctggagaactacaagaacctggtgtccctgggttaccagctcaccaagcctgatgtcatcctgaggctggaaaagggagaggaaccttggttggtggagagggaaatccaccaggaaactcaccctgactcagaaactgcctttgagatcaagagctctgtgtaa

SEQ ID NO: 12 - DNA, 828 bp, synthetic construct
  source 1..828: mol_type = other DNA
atggcccctaagaagaagagaaaagttggcatacatggagtccctgctgcaatggctgaaagacccttccaatgccggatttgcatgagaaacttctcctgcccctcccacctgaccagacacattaggactcacactggggagaagccttttgcctgtgacatctgtggaaggaagtttgctcagtcaggggacctcaccagacataccaagatccacacccccaacccccatagaagaactgacccctcccataagcctttccagtgccggatctgcatgaggaacttctccaagcatggaaacctgtcagaacacattagaacccacactggagaaaagcccttcgcctgtgatatctgtggtagaaagtttgccaagagatgcaacctgaggtgtcacaccaaaatccacactggcagccagagcccattccaatgcagaatctgcatgcgcaagtttgccagacagttcaacaggcaccagcacactaagatccacctgagacagaaagatgcagccaggggttcaggaatggatgccaagtccctcactgcctggtccaggaccctggtcaccttcaaggatgtctttgtggacttcaccagagaagagtggaagctcttggacactgcccagcaaattgtgtacaggaatgtgatgctggagaactacaagaacctggtgtccctgggctaccagctcaccaagcctgatgtgatcctgaggctggaaaagggagaggaaccctggcttgtggagagggaaattcaccaggaaacccaccctgactctgagactgcctttgagatcaagagctcagtgtaa

SEQ ID NO: 13 - DNA, 828 bp, synthetic construct
  source 1..828: mol_type = other DNA
atggcccctaagaagaagagaaaagttggcattcatggagtccctgcagctatggcagaaagaccattccagtgcagaatttgcatgagaaacttctcctgcccttcccacctgaccaggcatatcaggacccacactggggagaagccctttgcctgtgatatctgtggtagaaagtttgcccagtcaggggacctgaccagacacaccaaaattcacacccccaacccccatcggagaactgacccctcccataagcccttccagtgccggatctgcatgaggaacttcagcaagcatggaaacctctcagagcacatcagaactcacactggagaaaagcccttcgcctgtgacatctgtggaaggaagtttgccaagagatgcaacctgagatgccacactaagatccacactggtagccagtcccccttccaatgccgcatctgcatgcgcaagtttgctagacagttcaacaggcaccagcacaccaagatccacctgaggcagaaagatgcagccagaggctcaggaatggatgccaagtccctcactgcctggtcccggacccttgtgaccttcaaggatgtctttgtggacttcaccagagaagagtggaagctcctggacactgctcagcaaattgtgtacaggaatgtcatgctggagaactacaagaacctggtgtccctgggctaccaactcaccaagcctgatgtgatcctgaggctggaaaagggagaagaaccttggttggtggagagggaaattcaccaggaaacccaccctgactcagagactgcctttgagatcaagagctctgtgtaa

SEQ ID NO: 14 - DNA, 828 bp, synthetic construct
  source 1..828: mol_type = other DNA
atggcccctaagaagaagagaaaagttggcattcatggagtccctgcagctatggcagaaagacccttccaatgccggatctgcatgcggaacttctcctgcccctcccacctgaccagacacatcaggacccacactggagaaaagccctttgcttgtgacatctgtggtcggaagtttgcccagtcaggagacttgaccaggcataccaagattcacacccccaacccccatagaagaactgacccctcccacaagcccttccagtgccgcatctgcatgagaaacttcagcaagcatggcaacctgtcagaacacatcagaactcatactggagagaagccttttgcctgtgacatttgtggaagaaagtttgccaagagatgcaacctgaggtgccacaccaagatccacactggtagccagagcccattccagtgcagaatttgcatgaggaagtttgctaggcagttcaacaggcaccagcacaccaaaatccacctgaggcagaaagatgcagccaggggctctgggatggatgccaagtccctcactgcctggtccagaacccttgtgaccttcaaggatgtgtttgtggacttcaccagagaagagtggaagctcctggacactgcccagcaaattgtctacagaaatgtcatgctggagaactacaagaacctggtgtccctgggataccagctcaccaagcctgatgtgatcctccgcctggaaaagggggaagaaccttggttggtggagagggaaatccaccaagagactcaccctgactcagagactgcctttgagatcaagtcctcagtgtaa

SEQ ID NO: 15 - DNA, 828 bp, synthetic construct
  source 1..828: mol_type = other DNA
atggcccctaagaagaagagaaaagttggtatacatggagtccctgctgcaatggctgaaagacccttccagtgcagaatttgtatgagaaacttctcctgcccttcccacctgaccagacatatcaggactcacactggggaaaagccctttgcctgtgacatctgtggtagaaagtttgctcagtcaggggacctcaccagacacaccaagatccacacccccaacccccatagaagaactgacccctcccataagccattccaatgcagaatttgcatgaggaacttctccaagcatggcaacctgtcagaacacatcaggacccacactggagagaagccttttgcatgtgatatctgtggaaggaagtttgccaagagatgcaacttgagatgccacactaagattcacactggcagccagagcccattccagtgcaggatctgcatgagaaaatttgccagacagttcaacagacaccaacacaccaaaatccacctgaggcagaaagatgcagccaggggatctggaatggatgccaagtccctcactgcctggagcagaaccctggtcaccttcaaggatgtgtttgtggacttcaccagggaagagtggaagctcctggacactgcccagcaaattgtgtacaggaatgtgatgctggagaactacaagaacctggtgtccctgggctaccagctcaccaagcctgatgtcatcctgaggctggaaaagggagaggaaccctggcttgtggagagagagattcaccaggaaacccaccctgactcagaaactgcctttgagatcaagtcctcagtgtaa

SEQ ID NO: 16 - DNA, 828 bp, synthetic construct
  source 1..828: mol_type = other DNA
atggcccccaagaaaaagagaaaagttggcattcatggagtccctgctgcaatggctgaaagaccattccaatgcaggatctgcatgaggaacttcagctgcccctcccacctgaccaggcacattaggactcacactggagaaaagccctttgcctgtgacatctgtggcaggaagtttgcccagtcaggggacctcaccagacataccaagatccacacccccaaccctcatagaagaactgacccttcccacaagccattccagtgcagaatctgcatgagaaacttctccaagcatggaaacctgtcagagcacatcaggacccacactggggaaaagccttttgcttgtgacatttgtggtagaaaatttgccaagaggtgcaacctgagatgccacaccaagattcacactggatcccaaagccccttccagtgcagaatttgcatgagaaagtttgccaggcagttcaacaggcaccagcacactaagatccacctgagacagaaagatgcagccagaggctcaggaatggatgccaagagcctcactgcctggtccagaacccttgtgaccttcaaggatgtctttgtggacttcaccagagaagagtggaagctcctggacactgcccagcaaattgtgtacaggaatgtgatgctggagaactacaagaacctggtgtcactgggttaccagctcaccaagcctgatgtcatcctgagactggaaaagggagaggaaccctggttggtggagagagagatccatcaggaaacccaccctgactcagaaactgcctttgagatcaagtcctctgtgtaa

SEQ ID NO: 17 - DNA, 828 bp, synthetic construct
  source 1..828: mol_type = other DNA
atggcccctaagaagaagagaaaagttggcattcatggagtgccagcagcaatggctgagaggccattccagtgcagaatctgtatgaggaacttctcctgcccctcccacctcacaagacacatcaggacccatactggagagaagccctttgcctgtgacatctgtggtagaaagtttgcccagtcaggggacctgaccagacacactaagattcacacccccaacccacataggaggactgacccctcccataagcccttccagtgtagaatctgcatgaggaatttctccaagcatggcaacctgtcagaacacatcagaacccacactggggaaaagccttttgcttgtgatatctgtggaaggaaatttgccaagaggtgcaaccttagatgccataccaaaattcacactggaagccagagccccttccaatgtagaatttgcatgagaaaatttgccagacaattcaacagacaccagcacaccaagatccacctgagacaaaaggatgcagccagaggctctggaatggatgccaagagcctcactgcctggagcaggaccctggtcaccttcaaggatgtgtttgtggacttcaccagagaagagtggaagctgctggacactgcccagcaaattgtgtacagaaatgtcatgctggagaactacaagaacttggtgtccctgggttaccagctcaccaagcctgatgtgatcctgaggctggaaaagggagaggaaccttggttggtggagagggaaatccaccaggaaacccaccctgactcagaaactgcctttgagatcaagtccagtgtctaa

SEQ ID NO: 18 - DNA, 828 bp, synthetic construct
  source 1..828: mol_type = other DNA
atggcccccaagaagaaaagaaaagttggcattcatggggtgcctgctgcaatggcagaaaggccattccagtgcagaatctgcatgaggaacttctcctgcccctcccacctgaccagacacattagaacccacactggggagaagccctttgcctgtgacatctgtggcagaaaatttgcccagtctggggacctcaccagacacactaagattcacacccccaacccacatagaagaactgaccccagccacaagcccttccagtgtagaatctgtatgagaaacttcagcaagcatggaaacctgtcagagcacatcaggacccatactggagagaagccttttgcttgtgatatctgtggaaggaagtttgccaagaggtgcaacctgagatgccacaccaagatccacactggttcccaatccccattccaatgcagaatttgcatgagaaagtttgcaagacagttcaacaggcaccagcatactaagatccacctgaggcagaaagatgctgccaggggttcaggaatggatgccaagtccctcactgcctggagcagaacccttgtgaccttcaaggatgtctttgtggacttcacaagggaagagtggaagctcctggacactgcccagcaaattgtgtacaggaatgtcatgctggagaactacaagaaccttgtgtccctgggctaccagctgaccaagcctgatgtgatcctgagactggaaaagggagaggaaccttggttggtggagagggaaatccaccaggaaacccaccctgactcagagactgcctttgaaatcaagtcctcagtgtaa

SEQ ID NO: 19 - DNA, 828 bp, synthetic construct
  source 1..828: mol_type = other DNA
atggcccccaagaaaaagagaaaagtgggcattcatggagtgcctgcagctatggctgaaaggccattccagtgtagaatctgcatgagaaacttctcctgcccttcccacctgaccagacacatcaggacccacactggagagaaaccctttgcttgtgacatttgtggtagaaagtttgcacagtcaggggacctcaccaggcataccaagatccacaccccaaacccccatagaagaactgacccctcccataagcccttccagtgcagaatctgtatgaggaacttcagcaagcatggaaacctctcagagcatatcagaactcacactggggagaagccctttgcctgtgacatctgtgggaggaagtttgccaagagatgcaacctcagatgccacactaagattcacactggcagccagtccccattccaatgcagaatttgcatgaggaaatttgccagacaattcaataggcaccagcacaccaagattcacctgaggcagaaggatgctgccagaggctcaggaatggatgccaagtccctgactgcctggtccaggaccctggtcaccttcaaggatgtgtttgtggacttcaccagagaagagtggaagctgctggacacagcccagcaaattgtgtacaggaatgtcatgctggagaactacaagaacctggtgtccctgggctaccagcttaccaagcctgatgtcatcctgagactggaaaagggagaggaaccttggttggtggagagggaaatccaccaggaaacccaccctgactcagaaactgcctttgagatcaagagctctgtgtaa

SEQ ID NO: 20 - DNA, 828 bp, synthetic construct
  source 1..828: mol_type = other DNA
atggcccccaagaagaagaggaaagttggcattcatggagtgcctgctgcaatggctgagaggccattccagtgcagaatctgtatgaggaacttcagctgccccagccacctgaccagacacatcaggacccacactggagaaaagccctttgcctgtgacatttgtgggagaaaatttgcccagtcaggggacctcaccaggcacaccaagatccatacccccaacccacatagaagaactgacccttcccacaagcccttccaatgtagaatctgcatgagaaacttctccaagcatggaaacctctcagagcacattagaacccatactggggagaagccttttgcttgtgacatctgtggtagaaagtttgccaagaggtgcaacctgagatgccacactaagatccacactggtagccaaagcccattccaatgcagaatttgcatgaggaagtttgcaagacagttcaacagacaccagcacaccaaaatccacctgaggcagaaagatgcagccagaggctcaggaatggatgccaagtccttgacagcctggtccaggaccctggtcaccttcaaggatgtgtttgtggacttcaccagagaagagtggaagctgctggacactgcccagcaaattgtgtacaggaatgtcatgctggaaaactacaagaaccttgtgtccctgggctaccagctcaccaagcctgatgtgatcctgagactggaaaagggagaggaaccctggcttgtggagagggaaatccaccaggaaacccaccctgactctgagactgcctttgagatcaagtccagtgtctaa

SEQ ID NO: 21 - DNA, 828 bp, synthetic construct
  source 1..828: mol_type = other DNA
atggcccccaagaagaaaaggaaagttggcattcatggagtgcctgcagcaatggctgaaaggcccttccaatgcagaatctgtatgagaaacttctcctgcccctcccatctgactaggcacatcaggacccacactggagaaaagccatttgcctgtgacatttgtggtagaaagtttgcccagagtggagatctcaccagacacactaagatccacacccccaacccccatagaagaactgacccttcccacaagccattccagtgcaggatttgcatgagaaatttcagcaagcatgggaacctgtcagagcacatcagaacccatactggggaaaagccctttgcttgtgacatctgtggaagaaaatttgccaagaggtgcaacctgagatgccacaccaaaattcacacaggctcccagtccccattccaatgtagaatctgcatgaggaagtttgctaggcagttcaacaggcaccagcacaccaagatccacctgaggcagaaagatgcagccagaggctctggaatggatgccaagtccctcactgcctggagcagaaccctggtcaccttcaaggatgtgtttgtggacttcaccagagaagagtggaagctcctggacactgcccagcaaattgtgtacaggaatgtcatgcttgagaactacaagaacctggtgtccctgggctaccagttgaccaagcctgatgtgatcctgagactggaaaagggagaggaaccttggcttgtggagagagagatccaccaggaaacccaccctgactcagaaactgcctttgagatcaagagctcagtgtaa

SEQ ID NO: 22 - DNA, 828 bp, synthetic construct
  source 1..828: mol_type = other DNA
atggcccccaagaagaagagaaaagtgggcattcatggagtgcctgctgcaatggctgaaaggccattccagtgtagaatctgtatgaggaacttctcctgcccttcccacctgaccagacacattaggacccatactggagagaagccctttgcctgtgacatctgtggcaggaagtttgcccagtcaggggacctcactaggcacaccaagatccacacccccaacccacatagaagaactgaccccagccacaagccattccaatgcagaatttgtatgagaaacttctccaagcatggcaacctgtcagaacacatcaggacccacactggggagaagccttttgcttgtgacatttgtggtagaaaatttgccaagaggtgcaacctgagatgccacaccaaaatccacactggaagccagagccccttccagtgcagaatctgcatgagaaagtttgcaagacagttcaacagacatcaacacacaaagatccacctgaggcagaaagatgcagccaggggttctggaatggatgccaagtccctcactgcctggagcaggaccctggtcaccttcaaggatgtctttgtggacttcaccagagaagagtggaagctcctggacactgcccagcaaattgtgtacagaaatgtcatgctggagaactacaagaacctggtgtccttgggctaccagctcaccaagcctgatgtgatcctgagactggaaaagggagaggaaccctggcttgtggagagggaaatccaccaggaaacccaccctgactcagagactgcctttgaaatcaagtcctcagtgtaa

SEQ ID NO: 23 - DNA, 741 bp, synthetic construct
  source 1..741: mol_type = other DNA
atggcccccaagaaaaagcggaaagtgggcatccacggggtacccgccgctatggctgagaggcccttccagtgtcgaatctgcatgcgtaacttcagttccccggagcagctgtcccgccacatccgcacccacaccggcgagaagccttttgcctgtgacatttgtgggaggaaatttgcccagtggtccacccgcaagcgccataccaagatacacacgccgaacccgcaccgccgcaccgacccgtcccacaagcccttccagtgtcgaatctgcatgcgtaacttcagtaagcagggcaacctggtggagcacatccgcacccacaccggcgagaagccttttgcctgtgacatttgtgggaggaaatttgccaagcgctgtaacctgcgctgtcataccaagatacacctgcgccaaaaagatgcggcccggggatccggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagtttaa

SEQ ID NO: 24 - DNA, 741 bp, synthetic construct
  source 1..741: mol_type = other DNA
atggcacctaagaaaaagagaaaagtgggaattcatggagtaccagcagctatggcagaaagaccattccagtgcagaatctgcatgagaaacttctccagccctgaacagctgtccagacatatcaggacccatacaggggagaaaccctttgcctgtgacatctgtggtagaaagtttgcccaatggtccaccagaaagaggcacaccaagatccacacccccaacccacataggagaactgaccctagccacaagcccttccagtgtagaatttgcatgaggaacttcagcaaacagggcaacctggtggagcacatcagaacccacactggagaaaagccctttgcttgtgacatttgtggaaggaagtttgccaagaggtgcaacctgagatgccacactaagatccacctgagacaaaaggatgctgccaggggctctgggatggatgccaagtccctcactgcctggagcaggaccctggtcaccttcaaggatgtgtttgtggacttcaccagagaagagtggaagctcctggacactgcccagcaaattgtgtacaggaatgtcatgttggagaactacaagaacctggtgtccctgggctaccagctcaccaagcctgatgtgatcctgaggctggaaaagggagaagaaccctggcttgtggagagagagatccaccaggaaacccaccctgactcagagactgcctttgagatcaagtcctcagtctaa

SEQ ID NO: 25 - DNA, 741 bp, synthetic construct
  source 1..741: mol_type = other DNA
atggcccctaagaagaagagaaaagttggtattcatggagtccctgcagcaatggcagaaagaccattccaatgcagaatctgcatgagaaacttctcctcccctgaacaactgtccagacacatcaggacccatactggggagaagccctttgcctgtgacatctgtggaaggaagtttgcccagtggagcaccaggaaaaggcacaccaagatccacaccccaaacccccatagaagaactgaccccagccacaagccattccagtgcagaatttgcatgaggaacttcagcaaacaggggaacctggtggagcacatcagaacccacactggagaaaagccttttgcttgtgacatttgtggtagaaagtttgccaagaggtgcaacctgaggtgccataccaagattcacttgagacagaaagatgctgccaggggctcaggaatggatgccaagtccctcactgcctggagcagaaccctggtcaccttcaaggatgtgtttgtggacttcaccagagaagagtggaagctcctggacactgcccagcaaattgtgtacaggaatgtcatgctggaaaactacaagaacctggtgtccctgggctaccagctcaccaagcctgatgtgatcctgaggctggaaaagggagaggaaccctggcttgtggagagagagatccaccaggaaacccaccctgactcagagacagcctttgagatcaagtcctctgtctaa

SEQ ID NO: 26 - DNA, 741 bp, synthetic construct
  source 1..741: mol_type = other DNA
atggcaccaaagaaaaagagaaaagtgggaattcatggagtaccagcagcaatggctgaaagacccttccaatgtagaatttgcatgaggaacttcagcagccctgaacagctctcaaggcatatcagaacccacactggggagaagccttttgcctgtgacatctgtggcaggaagtttgcccagtggagcaccaggaaaaggcacaccaagatccatacccccaacccccatagaagaacagacccttcccacaagccattccagtgcagaatctgcatgagaaacttcagcaaacagggcaacctggtggaacacatcaggactcacactggagaaaagccctttgcttgtgacatttgtgggagaaagtttgccaagaggtgcaacctgagatgccacactaagatccacctgaggcagaaggatgctgccagaggttctggaatggatgccaagtccctcactgcctggtccagaacccttgtgaccttcaaggatgtgtttgtggacttcaccagagaagagtggaagctcctggacactgcccagcaaattgtgtacaggaatgtcatgctggagaactacaagaacctggtgtccttgggctaccagctgaccaagcctgatgtcatcctgaggctggaaaagggagaggaaccctggcttgtggagagagagatccaccaggaaacccaccctgactcagagactgcctttgagatcaagtcctcagtctaa

SEQ ID NO: 27 - DNA, 741 bp, synthetic construct
  source 1..741: mol_type = other DNA
atggcccctaagaagaagagaaaagttggtatacatggagtccctgctgcaatggctgaaagaccattccagtgcagaatttgcatgagaaacttctcctcccctgaacaactgtccaggcacattaggacccacactggagagaagccctttgcttgtgacatctgtggtagaaagtttgcccagtggagcaccaggaaaaggcacaccaaaatccacaccccaaacccccatagaagaactgacccctcccataagcccttccagtgtagaatctgcatgaggaacttcagcaaacaggggaacctggtggaacacatcaggacccatactggggagaagccttttgcctgtgacatttgtggaaggaagtttgccaagagatgcaacctgagatgccacaccaagatccacctgagacaaaaggatgcagccagaggctcaggaatggatgccaagtccctcactgcctggagcagaaccctggtcaccttcaaggatgtgtttgtggacttcactagagaagagtggaagctcctggacactgcccagcaaattgtgtacaggaatgtcatgttggagaactacaagaacctggtgtccctgggctaccagctcaccaagcctgatgtgatcctgaggctggaaaagggagaggaaccctggcttgtggagagggaaatccaccaggaaacccacccagactcagagacagcctttgagatcaagtcctctgtctaa

SEQ ID NO: 28 - DNA, 741 bp, synthetic construct
  source 1..741: mol_type = other DNA
atggccccaaagaaaaagagaaaagtaggcattcatggagtcccagcagcaatggcagaaagaccattccagtgcagaatctgcatgagaaacttctcctcccctgaacaattgtccaggcacatcaggacccacacaggggagaagccctttgcttgtgacatctgtggtagaaagtttgcccagtggtccaccagaaagagacacaccaagatccatacccccaacccccatagaagaactgaccctagccacaagcccttccagtgtagaatttgcatgaggaacttctccaaacaagggaacctggtggaacatatcaggactcacactggagagaagccttttgcctgtgacatttgtgggaggaagtttgccaagaggtgcaacctgagatgccacactaagatccacctgaggcagaaagatgctgccaggggctcaggaatggatgccaagtccctcactgcctggagcagaaccctggtcaccttcaaggatgtgtttgtggacttcaccagagaagagtggaagctcctggacactgcccagcaaattgtgtacaggaatgtcatgctggagaactacaagaacctggtgtccctgggctaccagctcaccaagcctgatgtgatcctgaggctggaaaagggagaggaaccctggcttgtggagagagaaatccaccaggaaacccaccctgactctgagactgcctttgagatcaagagctcagtctaa

SEQ ID NO: 29 - DNA, 741 bp, synthetic construct
  source 1..741: mol_type = other DNA
atggcacctaagaaaaagagaaaagtgggcatacatggagtcccagcagcaatggcagaaagaccattccagtgcagaatctgcatgagaaacttctcctcccctgaacaactgtccagacacatcaggactcatactggggaaaagccctttgcctgtgacatctgtggtagaaagtttgcccagtggtccactaggaaaaggcacaccaagatccacacccccaacccacatagaagaactgacccctcccacaagcccttccaatgtagaatttgcatgaggaacttcagcaaacagggcaacctggtggagcatatcaggacccacactggggagaagccttttgcttgtgacatttgtggaaggaagtttgccaagagatgcaacctcagatgccacactaagatccacctgaggcagaaggatgctgccaggggttctggaatggatgccaagtccctcactgcctggagcagaacccttgtgaccttcaaggatgtgtttgtggacttcaccagagaagagtggaagctgctggacacagcccaacagattgtgtacaggaatgtcatgctggagaactacaagaacttggtcagcctgggctaccagctcaccaagcctgatgtgatcctgagactggaaaagggagaggaaccctggcttgtggagagggaaatccaccaggaaacccaccctgactcagagactgcctttgagattaagagctcagtctaa

SEQ ID NO: 30 - DNA, 17 bp, synthetic construct
  source 1..17: mol_type = other DNA
agtttggagggtttctt

SEQ ID NO: 31 - DNA, 22 bp, synthetic construct
  source 1..22: mol_type = other DNA
tcgactaaagcaggatttcagg

SEQ ID NO: 32 - DNA, 17 bp, synthetic construct
  source 1..17: mol_type = other DNA
agtttggagggtttctc

SEQ ID NO: 33 - DNA, 17 bp, synthetic construct
  source 1..17: mol_type = other DNA
tttgtggacttcaccag

SEQ ID NO: 34 - DNA, 18 bp, synthetic construct
  source 1..18: mol_type = other DNA
gttctccagcatcacatt

SEQ ID NO: 35 - DNA, 18 bp, synthetic construct
  source 1..18: mol_type = other DNA
tgtatttgtggacttcac

SEQ ID NO: 36 - DNA, 17 bp, synthetic construct
  source 1..17: mol_type = other DNA
tgctgagcagtgtccag

SEQ ID NO: 37 - protein, 275 aa, synthetic construct
EMAHIPKRKTHKTRGKEVGKIPHFGACVDPIACAGMRAKEFRAPKFRQCCRNILCRMCRHNTFKSICHTPGSSHQLSTPRFHQICRRTIHTCGMERKKPFFAARCQDFINCRGHRQKHFTAKQISHLGRDQLKTDRAHATRKGISHGTMPDNAPHKRSLRTTADPWSSHRKTLPFVQTCFRKIDCVMFRVNDFFSTKRHEGENWLKSLLDTAQQIVYRNVMLENYKNLVSLGYQLTKPDVILRLEKGEEPWLVEREIHQETJPDSETAFEIKSSV

SEQ ID NO: 38 - protein, 247 aa, synthetic construct
EMAHIPKRKTHKTRGKEVGKIPHFGACVDPIACAGMRAKEFRAPKFRQCCRNILCRMCRHNTFKSISHLPREQQKLDSARAHRIGRSTGHTMGDEAKKPSFLATCADWISCRGTRLKVFTAFQKWDSVTFRVKDRFHTTRKEIEHWTKPLNLPDHTRARQTQDIPVSYHRKNPVFMQLCERNIYCKMNRLNVFSSLKGQYGQNLLTVKPDVILRLEKGEEPWLVEREIHQETHPDSETAAFEIKSSV

(19) United States
(12) Patent Application Publication
(10) Pub. No.: US 2026/0116932 A1
(43) Pub. Date: Apr. 30, 2026

(54) HTT REPRESSORS AND USES THEREOF

(71) Applicant: TAKEDA PHARMACEUTICAL COMPANY LIMITED, Osaka (JP)

(72) Inventors: Simon MOORE, Cambridge, MA (US); Gabriele PROETZEL, Cambridge, MA (US); Khanh DUONG, Cambridge, MA (US); Hidehisa IWATA, Fujisawa-shi (JP)

(21) Appl. No.: 19/429,445

(22) Filed: Dec. 22, 2025

Related U.S. Application Data
(63) Continuation of application No. PCT/IB2024/056361, filed on Jun. 28, 2024.
(60) Provisional application No. 63/511,437, filed on Jun. 30, 2023.

(51) Int. Cl. C07K 14/47; A61K 38/17; A61K 48/00; A61P 25/28; C12N 15/86

(52) U.S. Cl. CPC C07K 14/4702; A61K 38/1709; A61K 48/005; A61P 25/28; C12N 15/86; C12N 2750/14143

(57) ABSTRACT

Disclosed herein are improved methods and compositions for diagnosing, preventing and/or treating Huntington's Disease. Among other things, provided herein is a gene therapy construct encoding a non-naturally occurring codon-optimized transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK), ubiquitin C (UBC), an EFS, or an EF1alpha promoter.

### CROSS-REFERENCE TO RELATED APPLICATIONS

This application is a continuation application of International Application No. PCT/IB2024/056361, filed on Jun. 28, 2024, which claims priority to U.S. Provisional Patent Application No. 63/511,437 filed on Jun. 30, 2023, the contents of both of which are herein incorporated by reference in entireties for all purposes.

### INCORPORATION BY REFERENCE OF SEQUENCE LISTING

The instant application contains a Sequence Listing which has been submitted electronically in XML format and is hereby incorporated by reference in its entirety. Said XML copy, created on Jun. 19, 2024, is named MIL-037WO1_SL.xml and is 50,328 bytes in size.

### BACKGROUND

Huntington's Disease (HD), also known as Huntington's Chorea, is a progressive disorder of motor, cognitive and psychiatric disturbances. The mean age of onset for this disease is 35-44 years, although in about 10% of cases, onset occurs prior to age 21, and the average lifespan post-diagnosis of the disease is 15-18 years. Prevalence is about 3 to 7 among 100,000 people of western European descent.

Huntington's Disease is an example of a trinucleotide repeat expansion disorder and was first characterized in the early 1990s (see Di Prospero and Fischbeck (2005) Nature Reviews Genetics 6:756-765). These disorders involve the localized expansion of unstable repeats of sets of three nucleotides and can result in loss of function of the gene in which the expanded repeat resides, a gain of toxic function, or both. Trinucleotide repeats can be located in any part of the gene, including non-coding and coding gene regions. Repeats located within the coding regions typically involve either a repeated glutamine encoding triplet (CAG) or an alanine encoding triplet (CGA). Expanded repeat regions within non-coding sequences can lead to aberrant expression of the gene while expanded repeats within coding regions (also known as codon reiteration disorders) may cause mis-folding and protein aggregation.

The exact cause of the pathophysiology associated with the aberrant proteins is often not known. Typically, in the wild-type genes that are subject to trinucleotide expansion, these regions contain a variable number of repeat sequences in the normal population, but in the afflicted populations, the number of repeats can increase from a doubling to a log order increase. In HD, repeats are inserted within the N terminal coding region of the gene encoding the large cytosolic protein Huntingtin (HTT). Normal HTT alleles contain 15-24 CAG repeats (“CAG” repeats disclosed as SEQ ID NO: 23), while alleles containing 36 or more repeats can be considered potentially HD causing alleles and confer risk for developing the disease. Alleles containing 36-39 repeats are considered incompletely penetrant, and those individuals harboring those alleles may or may not develop the disease (or may develop symptoms later in life) while alleles containing 40 repeats or more are considered completely penetrant. In fact, no persons containing HD alleles with this many repeats have been reported to be asymptomatic. Those individuals with juvenile onset HD (<21 years of age) are often found to have 60 or more CAG repeats.

In addition to an increase in CAG repeats, it has also been shown that HD can involve +1 and +2 frameshifts within the repeat sequences such that the region will encode a poly-serine polypeptide (encoded by AGC repeats in the case of a +1 frameshift) track rather than poly-glutamine (Davies and Rubinsztein (2006) Journal of Medical Genetics 43:893-896).

In HD, the mutant HTT (m/77) allele is usually inherited from one parent as a dominant trait. Any child born of a HD patient has a 50% chance of developing the disease if the other parent was not afflicted with the disorder. In some cases, a parent may have an intermediate HD allele and be asymptomatic while, due to repeat expansion, the child manifests the disease. In addition, the HD allele can also display a phenomenon known as anticipation, wherein increasing severity or decreasing age of onset is observed over several generations due to the unstable nature of the repeat region during spermatogenesis.

Furthermore, trinucleotide expansion in HTT leads to neuronal loss in the medium spiny gamma-aminobutyric acid (GABA) projection neurons in the striatum, with neuronal loss also occurring in the neocortex. Medium spiny neurons that contain enkephalin and that project to the external globus pallidum are more involved than neurons that contain substance P and project to the internal globus pallidum. Other brain areas greatly affected in people with Huntington's disease include the substantia nigra, cortical layers 3, 5, and 6, the CA1 region of the hippocampus, the angular gyrus in the parietal lobe, Purkinje cells of the cerebellum, lateral tuberal nuclei of the hypothalamus, and the centromedialparafascicular complex of the thalamus (Walker (2007) Lancet 369:218-228).

The role of the normal HTT protein is poorly understood, but it may be involved in neurogenesis, apoptotic cell death, and vesicle trafficking. In addition, there is evidence that wild-type HTT stimulates the production of brain-derived neurotrophic factor (BDNF), a pro-survival factor for striatal neurons. It has been shown that progression of HD correlates with a decrease in BDNF expression in mouse models of HD (Zuccato et al. (2005) Pharmacological Research 52 (2): 133-139), and that delivery of either BDNF or glial cell line-derived neurotrophic factor (GDNF) via recombinant adeno-associated viral (rAAV) vector-mediated gene delivery may protect striatal neurons in mouse models of HD (Kells et al. (2004) Molecular Therapy 9 (5): 682-688).

Diagnostic and treatment options for HD are currently very limited. In terms of diagnostics, altered (mutant) HTT (mHTT) levels are significantly associated with disease burden score, and soluble mHTT species increase in concentration with disease progression. However, low-abundance mHTT is difficult to quantify in the patient CNS, which limits both study of the role in the neuropathobiology of HD in vivo, and precludes the demonstration of target engagement by HTT-lowering drugs. See, e.g., Wild et al. (2014) J Neurol Neurosurg Psychiatry 85: e4.

Current therapies include tetrabenazine (Xenazine) and deutetrabenazine (Austedo), approved by the Food and Drug Administration to suppress the symptom of involuntary jerking and writhing movements (chorea) associated with Huntington's disease. However, these drugs don't have any effect on the progression of the disease, and are associated with side effects including drowsiness, restlessness, and the risk of worsening or triggering depression or other psychiatric conditions. Antipsychotic drugs, such as haloperidol and fluphenazine, also suppress movements and may be beneficial in treating chorea. However, these drugs are also known to worsen involuntary contractions (dystonia), restlessness and drowsiness. Other drugs, such as olanzapine (Zyprexa) and aripiprazole (Abilify), have fewer side effects but are also known worsen symptoms in some patients.

However, there remains a need for methods for the diagnosis, treatment and/or prevention of Huntington's Disease, including for modalities that exhibit widespread delivery to the brain.

### SUMMARY

Disclosed herein are improved methods and compositions for diagnosing, preventing and/or treating Huntington's Disease. Described herein are non-naturally occurring zinc finger proteins (ZFPs) that bind to the CAG repeats domain of mHITT gene, including ZFPs comprising the recognition helix regions of the ZFPs designated ZFP46025 or ZFP45723 and codon-optimized variants of ZFPs. The present invention provides, among other things, a gene therapy construct comprising zinc finger proteins, for example, ZFP46025 or ZFP45723 as well as codon-optimized variants of ZFPs. Inventors used the ATUM tool to generate hundreds of codon-optimized sequences, which were then screened for various parameters, for example, remove potential splice sites, cryptic promoters, long repeats, and have reduced CpG (for example, less than 6 CpG), among other things. Through careful experimentation, inventors of the instant invention discovered codon-optimized sequences with higher expression and/or activity than parental ZFPs, providing improved methods and compositions for treating Huntington's Disease. ZFPs of the present invention are expressed under the control of promoters that are optimized for a favorable in vivo expression profile, for example, phosphoglycerate kinase 1 (PGK) and ubiquitin C (UBC). Without wishing to be bound by any particular theory, it is contemplated that use of exemplary PGK or UBC promoters, among others, for example, an EFS or an EF1alpha promoter, without limitation, prevent overexpression of ZFP, that triggers an immune response or silencing. Further, the present invention provides pharmaceutical compositions comprising viral (e.g., AAV-based, e.g., AAV5, AAV9, e.g., AAV comprising a capsid that penetrates the blood brain barrier, i.e., BBB-penetrant AAV) or non-viral (e.g., lipid nanoparticle, liposome-based) delivery of the gene therapy construct described herein. In some embodiments, delivery using BBB penetrating AAVs allows intravenous administration of ZFP-TFs of the present invention to the brain, reducing the challenges and risks of direct CNS administration. Also provided herein are methods and compositions for modifying (e.g., modulating expression of an HD HTT allele so as to prevent or treat Huntington Disease, including mHTT repressors (that repress mHTT transcripts and thus also repress mHTT protein expression). The compositions (e.g., mHTT repressors) described herein provide a therapeutic benefit in subjects, for example by reducing cell death, decreasing apoptosis, increasing cellular function (metabolism) and/or reducing motor deficiency in the subjects. Provided is a method of treating Huntington's disease by administering a composition comprising the gene therapy constructs described herein. Provided is a pharmaceutical composition comprising the gene therapy constructs and use of the pharmaceutical composition comprising ZFP-TFs described herein for treating Huntington's disease.

The methods and compositions of the present disclosure provide several advantages in gene therapy, for example, increased expression or reduced immunogenicity and increased safety, and provide improved methods for treating Huntington disease. In some aspects, provided herein is a gene therapy construct encoding a non-naturally occurring transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK) or a ubiquitin C (UBC) promoter, among others, including without limitation, an EFS, or an EF1alpha promoter.

In some embodiments, the ZFP comprises a recognition helix region designated ZFP46025 or ZFP45723. In some embodiments, the ZFP comprises a recognition helix region designated ZFP46025. In some embodiments, the ZFP comprises a recognition helix region designated ZFP45723.

In some embodiments, the ZFP is codon-optimized.

In some embodiments, the ZFP comprises a nucleotide sequence having at least 60% identity to any one of SEQ ID NO: 10-29.

In some embodiments, the ZFP comprises a nucleotide sequence having at least 65%, 70%, 75%, 80%, 85%, 90%, 95% or greater identity to any one of SEQ ID NO: 10-29.

In some embodiments, the ZFP-TF comprises a nucleotide sequence having 100% identity to any one SEQ ID NO: 10-29.

In some aspects, provided herein is a gene therapy construct comprising a non-naturally occurring codon optimized transcription factor (ZFP-TF) comprising a zinc-finger protein (ZFP) sequence and a sequence encoding a transcriptional repression domain, wherein the ZFP comprises a recognition helix region designated ZFP46025 or ZFP45723, and wherein the ZFP binds to a target site in a mutant HTT (mHTT) gene.

In some aspects, provided herein is a gene therapy construct comprising a non-naturally occurring codon-optimized transcription factor (ZFP-TF) comprising a zinc-finger protein (ZFP) sequence and a sequence encoding a transcriptional repression domain, wherein the ZFP-TF comprises a nucleotide sequence having at least 85% identity to any one of SEQ ID NO: 11-22 or SEQ ID NO: 24-29, and wherein the ZFP binds to a target site in a mutant HTT (mHTT) gene.

In some embodiments, the ZFP-TF comprises a nucleotide sequence having 90%, 95% or greater identity to any one of SEQ ID NO: 11-22 or SEQ ID NO: 24-29. In some embodiments, the ZFP-TF comprises a nucleotide sequence having 90% identity to any one of SEQ ID NO: 11-22 or SEQ ID NO: 24-29. In some embodiments, the ZFP-TF comprises a nucleotide sequence having 95% identity to any one of SEQ ID NO: 11-22 or SEQ ID NO: 24-29. In some embodiments, the ZFP-TF comprises a nucleotide sequence having greater than 90% identity to any one of SEQ ID NO: 11-22 or SEQ ID NO: 24-29. In some embodiments, the ZFP-TF comprises a nucleotide sequence having between 90%-100% identity to any one of SEQ ID NO: 11-22 or SEQ ID NO: 24-29.

In some embodiments, the ZFP-TF comprises 100% identity to any one of SEQ ID NO: 11-22 or SEQ ID NO: 24-29.

In some embodiments, the ZFP-TF expression is driven by a phosphoglycerate kinase 1 (PGK), a ubiquitin C (UBC), an EFS, or an EF1alpha promoter. In some embodiments, the ZFP-TF expression is driven by a phosphoglycerate kinase 1 (PGK) promoter. In some embodiments, the ZFP-TF expression is driven by a ubiquitin C (UBC) promoter. In some embodiments, the ZFP-TF expression is driven by an EFS promoter. In some embodiments, the ZFP-TF expression is driven by an EF1alpha promoter.

In some embodiments, the recognition helix region of the ZFP-TF comprises the amino acid sequence of one of SEQ ID NO: 1-5 or SEQ ID NO: 7-9.

In some embodiments, the target site comprises a CAG repeats domain of the mHTT gene.

In some embodiments, the target site recognizes a sequence comprising 70%, 75%, 80%, 85%, 90%, 95% or greater identity to SEQ ID NO: 6. In some embodiments, the target site recognizes a sequence comprising 70%-75%, 75%-80%, 80%-85%, 85%-90%, 90%-95% or greater identity to SEQ ID NO: 6. In some embodiments, the target site recognizes a sequence comprising 70% identity to SEQ ID NO: 6. In some embodiments, the target site recognizes a sequence comprising 75% identity to SEQ ID NO: 6. In some embodiments, the target site recognizes a sequence comprising 80% identity to SEQ ID NO: 6. In some embodiments, the target site recognizes a sequence comprising 85% identity to SEQ ID NO: 6. In some embodiments, the target site recognizes a sequence comprising 90% identity to SEQ ID NO: 6. In some embodiments, the target site recognizes a sequence comprising 95% identity to SEQ ID NO: 6.

In some embodiments, the target site recognizes a sequence comprising 100% identity to SEQ ID NO: 6.

In some embodiments, the ZFP-TF further comprises a sequence encoding a nuclear localization sequence (NLS).

In some embodiments, the NLS is SV40.

In some embodiments, the ZFP-TF further comprises inverted terminal repeats (ITRs) flanking the promoter. In some embodiments, the ZFP-TF further comprises inverted terminal repeats (ITRs) flanking the PGK promoter. In some embodiments, the ZFP-TF further comprises inverted terminal repeats (ITRs) flanking the UBC promoter. In some embodiments, the ZFP-TF further comprises inverted terminal repeats (ITRs) flanking the EFS promoter. In some embodiments, the ZFP-TF further comprises inverted terminal repeats (ITRs) flanking the EF1alpha promoter.

In some embodiments, the ZFP-TF further comprises a human growth hormone (hGH) poly adenylation signal.

In some embodiments, the gene therapy construct is delivered using a viral vector.

In some embodiments, the viral vector is adeno-associated virus (AAV), lentivirus or adenovirus. In some embodiments, the viral vector is adeno-associated virus (AAV). In some embodiments, the viral vector is lentivirus. In some embodiments, the viral vector is adenovirus. In some embodiments, the viral vector is a Virus-Like Particle (VLP).

In some embodiments, the gene therapy construct is delivered using a lipid nanoparticle (LNP) or liposome. In some embodiments, the gene therapy construct is delivered using a lipid nanoparticle (LNP). In some embodiments, the gene therapy construct is delivered using a liposome.

In some aspects, provided herein is a recombinant rAAV vector comprising a gene therapy construct encoding a non-naturally occurring transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK) or a ubiquitin C (UBC) promoter. In some aspects, provided herein is a recombinant rAAV vector comprising a gene therapy construct encoding a non-naturally occurring transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK) promoter. In some aspects, provided herein is a recombinant rAAV vector comprising a gene therapy construct encoding a non-naturally occurring transcription factor (ZFP-TF) comprising a zinc-finger protein (ZFP) sequence and a sequence encoding a transcriptional repression domain, wherein the ZFP-TF expression is driven by a ubiquitin C (UBC) promoter. In some aspects, provided herein is a recombinant rAAV vector comprising a gene therapy construct encoding a non-naturally occurring transcription factor (ZFP-TF) comprising a zinc-finger protein (ZFP) sequence and a sequence encoding a transcriptional repression domain, wherein the ZFP-TF expression is driven by an EFS promoter. In some aspects, provided herein is a recombinant rAAV vector comprising a gene therapy construct encoding a non-naturally occurring transcription factor (ZFP-TF) comprising a zinc-finger protein (ZFP) sequence and a sequence encoding a transcriptional repression domain, wherein the ZFP-TF expression is driven by an EF1alpha promoter.

In some aspects, provided herein is an rAAV vector comprising a gene therapy construct comprising a non-naturally occurring codon optimized transcription factor (ZFP-TF) comprising a zinc-finger protein (ZFP) sequence and a sequence encoding a transcriptional repression domain, wherein the ZFP comprises a recognition helix region designated ZFP46025 or ZFP45723, and wherein the ZFP binds to a target site in a mutant HTT (mHTT) gene.

In some aspects, provided herein is an rAAV vector comprising a gene therapy construct comprising a non-naturally occurring codon-optimized transcription factor (ZFP-TF) comprising a zinc-finger protein (ZFP) sequence and a sequence encoding a transcriptional repression domain, wherein the ZFP-TF comprises a nucleotide sequence having at least 85% identity to any one of SEQ ID NO: 11-22 or SEQ ID NO: 24-29, and wherein the ZFP binds to a target site in a mutant HTT (mHTT) gene.

In some embodiments, the rAAV vector is AAV1, AAV2, AAV5, AAV7, AAV9 or AAVrh10. In some embodiments, the rAAV vector is rAAV vector is AAV1. In some embodiments, the rAAV vector is rAAV vector is AAV2. In some embodiments, the rAAV vector is rAAV vector is AAV5. In some embodiments, the rAAV vector is rAAV vector is AAV7. In some embodiments, the rAAV vector is rAAV vector is AAV9. In some embodiments, the rAAV vector is rAAV vector is AAVrh10.

In some embodiments, the rAAV vector comprises a capsid protein that penetrates a blood brain barrier (BBB).

In some embodiments, the rAAV vector is VCAP-101, VCAP-102, 9P801, VCAP-100, VCAP-103, PAL1A, PAL1B, PAL1C, PAL2, CereAAV, Dyno bCAP1, AAV.CAP-B10, AAV.CAP-B20, AAV2-BRIN, AAV2-BR1, STAC-BBBX, or AAV-TT, or AAV-BI-hTFR1.

In some embodiments, provided herein is a lipid nanoparticle comprising a gene therapy construct described herein.

In some embodiments, provided herein is a pharmaceutical composition comprising a rAAV vector or a lipid nanoparticle.

In some embodiments, provided herein is a method of modulating expression of a mutant Huntington's Disease (mHTT) allele comprising administering a pharmaceutical composition provided herein.

In some aspects, provided herein is a method of modulating expression of a mutant Huntington's Disease HTT (mHTT) allele comprising administering an rAAV or a lipid nanoparticle comprising one or more gene therapy constructs encoding a non-naturally occurring transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK), a ubiquitin C (UBC), an EFS, or an EF1alpha promoter, wherein the ZFP binds to a target site in a mutant HTT (mHTT) gene, and wherein upon administration, expression of a mutant (mHTT) allele is reduced. In some aspects, provided herein is a method of modulating expression of a mutant Huntington's Disease HTT (mHTT) allele comprising administering an rAAV or a lipid nanoparticle comprising one or more gene therapy constructs encoding a non-naturally occurring transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK) promoter, wherein the ZFP binds to a target site in a mutant HTT (mHTT) gene, and wherein upon administration, expression of a mutant (mHTT) allele is reduced. In some aspects, provided herein is a method of modulating expression of a mutant Huntington's Disease HTT (mHTT) allele comprising administering an rAAV or a lipid nanoparticle comprising one or more gene therapy constructs encoding a non-naturally occurring transcription factor (ZFP-TF) comprising a zinc-finger protein (ZFP) sequence and a sequence encoding a transcriptional repression domain, wherein the ZFP-TF expression is driven by a ubiquitin C (UBC) promoter, wherein the ZFP binds to a target site in a mutant HTT (mHTT) gene, and wherein upon administration, expression of a mutant (mHTT) allele is reduced. In some aspects, provided herein is a method of modulating expression of a mutant Huntington's Disease HTT (mHTT) allele comprising administering an rAAV or a lipid nanoparticle comprising one or more gene therapy constructs encoding a non-naturally occurring transcription factor (ZFP-TF) comprising a zinc-finger protein (ZFP) sequence and a sequence encoding a transcriptional repression domain, wherein the ZFP-TF expression is driven by an EFS promoter, wherein the ZFP binds to a target site in a mutant HTT (mHTT) gene, and wherein upon administration, expression of a mutant (mHTT) allele is reduced. In some aspects, provided herein is a method of modulating expression of a mutant Huntington's Disease HTT (mHTT) allele comprising administering an rAAV or a lipid nanoparticle comprising one or more gene therapy constructs encoding a non-naturally occurring transcription factor (ZFP-TF) comprising a zinc-finger protein (ZFP) sequence and a sequence encoding a transcriptional repression domain, wherein the ZFP-TF expression is driven by an EF1alpha promoter, wherein the ZFP binds to a target site in a mutant HTT (mHTT) gene, and wherein upon administration, expression of a mutant (mHTT) allele is reduced.

In some aspects, provided herein is a method of treating Huntington's Disease comprising administering to a subject in need thereof, an rAAV or a lipid nanoparticle comprising one or more gene therapy constructs encoding a non-naturally occurring transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK), a ubiquitin C (UBC), an EFS, or an EF1alpha promoter, wherein the ZFP binds to a target site in a mutant HTT (mHTT) gene, and wherein upon administration, one or more symptoms associated with Huntington's disease is reduced or relieved. In some aspects, provided herein is a method of treating Huntington's Disease comprising administering to a subject in need thereof, an rAAV or a lipid nanoparticle comprising one or more gene therapy constructs encoding a non-naturally occurring transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK) promoter, wherein the ZFP binds to a target site in a mutant HTT (mHTT) gene, and wherein upon administration, one or more symptoms associated with Huntington's disease is reduced or relieved. In some aspects, provided herein is a method of treating Huntington's Disease comprising administering to a subject in need thereof, an rAAV or a lipid nanoparticle comprising one or more gene therapy constructs encoding a non-naturally occurring transcription factor (ZFP-TF) comprising a zinc-finger protein (ZFP) sequence and a sequence encoding a transcriptional repression domain, wherein the ZFP-TF expression is driven by a ubiquitin C (UBC) promoter, wherein the ZFP binds to a target site in a mutant HTT (mHTT) gene, and wherein upon administration, one or more symptoms associated with Huntington's disease is reduced or relieved. In some aspects, provided herein is a method of treating Huntington's Disease comprising administering to a subject in need thereof, an rAAV or a lipid nanoparticle comprising one or more gene therapy constructs encoding a non-naturally occurring transcription factor (ZFP-TF) comprising a zinc-finger protein (ZFP) sequence and a sequence encoding a transcriptional repression domain, wherein the ZFP-TF expression is driven by an EFS promoter, wherein the ZFP binds to a target site in a mutant HTT (mHTT) gene, and wherein upon administration, one or more symptoms associated with Huntington's disease is reduced or relieved. In some aspects, provided herein is a method of treating Huntington's Disease comprising administering to a subject in need thereof, an rAAV or a lipid nanoparticle comprising one or more gene therapy constructs encoding a non-naturally occurring transcription factor (ZFP-TF) comprising a zinc-finger protein (ZFP) sequence and a sequence encoding a transcriptional repression domain, wherein the ZFP-TF expression is driven by an EF1alpha promoter, wherein the ZFP binds to a target site in a mutant HTT (mHTT) gene, and wherein upon administration, one or more symptoms associated with Huntington's disease is reduced or relieved.

In some embodiments, the ZFP comprises a recognition helix region designated ZFP46025 or ZFP45723.

In some embodiments, the ZFP is codon-optimized.

In some embodiments, the ZFP-TF comprises a nucleotide sequence having at least 85% identity to any one of SEQ ID NO: 11-22 or SEQ ID NO: 24-29.

In some aspects, provided herein is a method of treating Huntington's disease comprising administering to a subject in need thereof a therapeutically effective amount of a pharmaceutical composition described herein.

In some embodiments, the one or more symptoms is cell death.

In some embodiments, the one or more symptoms is apoptosis.

In some embodiments, the one or more symptoms is motor deficiency.

In some embodiments, the administration is intrathecal, intracerebroventricular, intranasal or intravenous. In some embodiments, the administration is intrathecal. In some embodiments, the administration is intracerebroventricular. In some embodiments, the administration is intranasal. In some embodiments, the administration is intravenous.

In some embodiments, the administration is via focused ultrasound.

In some embodiments, the administration is to the brain.

In some embodiments, the administration to the brain is to any one of striatum, cortex, caudate, putamen, thalamus, or globus pallidus regions. In some embodiments, the administration to the brain is to striatum. In some embodiments, the administration to the brain is to cortex. In some embodiments, the administration to the brain is to caudate region. In some embodiments, the administration to the brain is to putamen. In some embodiments, the administration to the brain is to thalamus. In some embodiments, the administration to the brain is to globus pallidus regions. In some embodiments, the administration is to caudate region and globus pallidus regions. In some embodiments, the administration is to at least one region of the brain. In some embodiments, the administration is to two or more regions of the brain. In some embodiments, the administration is to three or more regions of the brain. In some embodiments, the administration is to four or more regions of the brain. In some embodiments, the administration is to five or more regions of the brain. It is to be understood by a skilled person that the administration is to any number and any combination of regions listed in any order. As non-limiting examples, in some embodiments, the administration is to striatum, cortex, and caudate regions. In some embodiments, the administration is to cortex, caudate, and putamen regions. In some embodiments, the administration is to caudate, putamen, and thalamus regions. In some embodiments the administration is to putamen, thalamus, or globus pallidus regions. In some embodiments, the administration is to thalamus, globus pallidus, and striatum regions. In some embodiments, the administration is to globus pallidus, striatum, and cortex regions.

In some embodiments of the method, the administration is systemic.

In some embodiments of the method, the administration is to the central nervous system (CNS).

In some aspects, provided herein is a method of treating Huntington's Disease comprising administering to a subject in need thereof, an rAAV or a lipid nanoparticle comprising one or more gene therapy constructs described herein, wherein the rAAV is a BBB-penetrant rAAV, wherein the administration is intravenous, and wherein upon administration, one or more symptoms associated with Huntington's disease is reduced or relieved.

These and other aspects and embodiments are illustrative of the present disclosure, and non-limiting, as will be readily apparent to the skilled artisan in light of disclosure as a whole.

### BRIEF DESCRIPTION OF THE DRAWINGS

FIG. 1A, FIG. 1B and FIG. 1C: FIG. 1A depicts an outline of the Zinc Finger Proteins (ZFPs) including the nuclear localization signal, zinc finger (ZF) domains and KRAB transcriptional repressor domain from the KOX1 protein. FIG. 1B shows the amino acid sequence of ZFP46025 and FIG. 1C shows the amino acid sequence of ZFP45723 outlining the NLS, ZF and KRAB domains.

FIG. 2A, FIG. 2B, FIG. 2C and FIG. 2D: FIG. 2A is a diagram of the expression cassette containing a hybrid chicken beta-actin (CBh) promoter, a human growth hormone (hGH) poly adenylation signal (polyA) and a transgene that includes: ZFP46025 variants (parental and codon optimized variant sequences), a T2A self-cleaving peptide and eGFP. FIG. 2B shows a Western blot of HEK293 lysates probed using an antibody targeting the ZFP and GAPDH. FIG. 2C shows quantification of the mean Western blot band intensity normalized to GAPDH intensity, with values indicated above each bar. FIG. 2D shows quantification of ZFP protein expression as measured by GFP signal intensity analysis of transient transfected HEK293 cells.

FIG. 3A, FIG. 3B and FIG. 3C: FIG. 3A is a diagram of the expression cassette containing a hybrid chicken beta-actin (CBh) promoter, a human growth hormone (hGH) poly adenylation signal (polyA) and a transgene with either parental or codon optimized variants of ZFP46025. FIG. 3B is a Western blot of HEK293 lysates probed using an antibody targeting the ZFP and GAPDH. FIG. 3C shows quantification of the mean western blot band intensity normalized to GAPDH intensity, with values indicated above each bar.

FIG. 4A, FIG. 4B, FIG. 4C and FIG. 4D: FIG. 4A is a diagram of the recombinant genome with ITRs flanking a human ubiquitin-c (UBC) promoter, a transgene of the ZFP46025 variants, a human growth hormone (hGH) poly adenylation signal and a 1 kb stuffer sequence. FIG. 4B is a graph showing PCR mRNA quantification of ZFP46025 normalized to GAPDH, with values indicated above each bar. FIG. 4C shows immunocytochemistry based protein quantification of ZFP46025 product with values indicated above each bar. FIG. 4D is a graph showing PCR mRNA quantification of wild type and mutant HTT alleles normalized to GAPDH, with values indicated above each bar.

FIG. 5A, FIG. 5B, FIGS. 5C and 5D: FIG. 5A is a diagram of the recombinant genome with ITRs flanking a human phosphoglycerate kinase 1 (PGK) promoter, a transgene of the ZFP46025 variants, a human growth hormone (hGH) poly adenylation signal and a 1 kb stuffer sequence. FIG. 5B is a graph of PCR mRNA quantification of ZFP46025 normalized to GAPDH, with values indicated above each bar. FIG. 5C is a graph of immunocytochemistry based protein quantification of ZFP46025 product with values indicated above each bar. FIG. 5D is a graph of PCR mRNA quantification of wild type and mutant HTT alleles normalized to GAPDH, with values indicated above each bar.

FIGS. 6A, 6B, 6C and 6D: FIG. 6A is a diagram of the recombinant genome with ITRs flanking a human phosphoglycerate kinase 1 (PGK) promoter, a transgene of the ZFP46025 variants, a human growth hormone (hGH) poly adenylation signal and a 1 kb stuffer sequence. FIG. 6B is a graph of PCR mRNA quantification of ZFP46025 normalized to GAPDH, with values indicated above each bar. FIG. 5C is a graph of immunocytochemistry based protein quantification of ZFP46025 product with values indicated above each bar. FIG. 5D is a graph of PCR mRNA quantification of wild type and mutant HTT alleles normalized to GAPDH, with values indicated above each bar.

FIG. 7A, FIG. 7B and FIG. 7C: FIG. 7A is a diagram of the expression cassette containing a hybrid chicken beta-actin (CBh) promoter, a human growth hormone (hGH) poly adenylation signal (polyA) and a transgene with either parental or codon optimized variants of ZFP45723. FIG. 7B is a Western blot of HEK293 lysates probed using an antibody targeting the KOX region of the ZFP or GAPDH. FIG. 7C shows quantification of the mean western blot band intensity normalized to GAPDH intensity, with values indicated above each bar.

FIG. 8A, FIG. 8B and FIG. 8C: FIG. 8A is a diagram of the recombinant genome with ITRs flanking a human phosphoglycerate kinase 1 (PGK) promoter, a transgene of the ZFP45723 variants, a human growth hormone (hGH) poly adenylation signal and a 1 kb stuffer sequence. FIG. 8B shows PCR mRNA quantification of ZFP46025 normalized to GAPDH, with values indicated above each bar. FIG. 8C shows immunocytochemistry based protein quantification of ZFP46025 product with values indicated above each bar.

FIG. 9A, FIG. 9B and FIG. 9C: FIG. 9A is a vector genome (VG) analysis from striatum of AAV-treated Q175 mice by qPCR. FIG. 9B shows RNA analysis for transgene RNA expression in striatum of AAV-treated Q175 mice. FIG. 9C shows evaluation of mHTT RNA lowering in striatum of Q175 mice after AAV9 intra-striatal administration, calculated compared to vehicle control.

FIG. 10A-FIG. 10G: FIG. 10A is a vector genome (VG) analysis from striatum and cortex of AAV-treated Q175 mice by qPCR. FIG. 10B shows RNA analysis for transgene RNA expression in striatum and cortex of AAV-treated Q175 mice. FIG. 10C shows evaluation of mHTT RNA lowering in striatum and cortex of Q175 mice after AAV9 intravenous (IV) administration. FIG. 10D shows a graph quantifying levels of soluble mHTT in brain cortex as determined using a MSD immunoassay. FIG. 10E shows a graph quantifying levels of soluble mHTT in brain striatum as determined using a MSD immunoassay. FIG. 10F shows a graph quantifying levels of aggregated mHTT in brain cortex as determined using a MSD immunoassay. FIG. 10G shows a graph quantifying levels of aggregated mHTT in striatum as determined using a MSD immunoassay.

FIG. 11 depicts graphs quantifying ZFP expression, showing that codon optimized sequences SEQ ID NO: 15 (NH035) and SEQ ID NO: 16 (NH035) yield a lower expression compared to the non-codon optimized parental (SEQ ID NO: 10) NH014 AAV.

### DETAILED DESCRIPTION

Disclosed herein are compositions and methods for widespread CNS delivery of compositions for detecting, monitoring disease progression, treating and/or preventing Huntington's disease (HD). In some aspects, the compositions and methods described herein use AAV vectors (for example, BBB-penetrant AAV) for delivery of mHTT repressors, which provides for the spread of functional mHTT repressors beyond the site of delivery. The mHTT repressors (e.g., mHTT-modulating transcription factors, such as mHTT-modulating transcription factors comprising zinc finger proteins (ZFP TFs) modify the CNS such that the effects and/or symptoms of HD are reduced or eliminated, for example by reducing the aggregation of HTT in HD neurons, by increasing HD neuron energetics (e.g., increasing ATP levels), by reducing apoptosis in HD neurons and/or by reducing motor deficits in HD subjects.

Described herein are gene therapy constructs comprising non-naturally occurring transcription factors (ZFP-TFs) comprising these ZFPs operably linked to a transcriptional repression domain (e.g., KRAB, KOX, etc.) and optionally comprising additional elements such as a nuclear localization signal (NLS) and/or a promoter (e.g., a constitutive promoter such as a PGK promoter or a UBC promoter) driving expression of the ZFP-TF-encoding sequence (e.g., a ZFP-TF comprising the ZFP designated ZFP46025 or ZFP45723 further comprising a sequence encoding a transcriptional repression domain and optionally comprising a sequence encoding an NLS and/or a promoter driving expression of the ZFP-TF). In some embodiments, the promoter is flanked by inverted terminal repeats (ITRs). In some embodiments, the gene therapy construct further comprises a human growth hormone poly adenylation signal.

In certain embodiments, provided herein are one or more ZFP-TFs having the nucleotide sequence as shown in Table 3.

Described herein is a zinc finger protein transcription factor (ZFP-TF) comprising a zinc finger protein (ZFP) designated ZFP46025 or ZFP45723 or encoded by the sequence of ZFP46025 or ZFP45723 or codon-optimized variants thereof as shown in Table 3. Also described are one or more polynucleotides encoding one or more ZFP-TFs as described herein, in which the one or more polynucleotides may encode one or more of the same and/or different ZFP-TFs, optionally wherein the one or more polynucleotides comprise one or more rAAV vectors e.g., an rAAV comprising a sequence encoding one or more ZFP-TFs comprising the ZFP designated ZFP46025 or ZFP45723 or wherein the rAAV vector comprises a polynucleotide having the sequence shown in Table 3, optionally wherein one or more rAAV vectors further comprise additional elements such as a sequence encoding a nuclear localization signal (NLS) and, optionally, a promoter driving expression of the ZFP-TF, such as a constitutive promoter (e.g., PGK, UBC, EFS or EF1alpha).

Also described herein is a pharmaceutical composition comprising one or more ZFP-TFs, one or more polynucleotides and/or one or more rAAV vectors as described herein. Methods of modifying expression of an HTT gene, e.g., a mutant HTT (mHTT gene) in a cell, e.g., a neuronal cell in the brain, optionally in the striatum of a subject are also provided, the method comprising administering to the cell one or more ZFP-TFs, one or more polynucleotides, one or more rAAV vectors and/or a pharmaceutical composition as described herein to the cell of subject.

Methods of treating and/or preventing Huntington's Disease (HD) in a subject in need thereof are also provided, the method comprising administering one or more ZFP-TFs, one or more polynucleotides, one or more rAAV vectors and/or a pharmaceutical composition according as described herein to the subject in need thereof, optionally wherein the one or more ZFP-TFs, polynucleotides, rAAV vectors and/or pharmaceutical compositions are administered bilaterally to the striatum of the subject. Also provided is use of one or more ZFP-TFs, one or more polynucleotides, one or more rAAV vectors and/or a pharmaceutical composition as described herein for repression of mutant HTT (mHTT) expression in a subject in need thereof. Treatment and/or prevention of HD may involve reduction of mHTT aggregates and/or motor deficiencies in the subject. Furthermore, in any of the method or uses described herein the one or more ZFP-TFs, one or more polynucleotides, one or more rAAV vectors and/or pharmaceutical composition may be delivered to the brain of the subject, optionally bilaterally to the striatum of the subject at any dosages, including but not limited to at a dose of between 1×10⁷ and 1×10¹⁵ (or any value therebetween) vector genomes (vg) per striatum.

Thus, in one aspect, engineered (non-naturally occurring) mHTT repressors are provided. The repressors may comprise systems (e.g., zinc finger proteins) that modulate expression of a HD allele (e.g., mHTT). Engineered zinc finger proteins are non-naturally occurring zinc finger proteins whose DNA binding domains (e.g., recognition helices or RVDs) have been altered (e.g., by selection and/or rational design) to bind to a pre-selected target site. Any of the zinc finger proteins described herein may include 1, 2, 3, 4, 5, 6 or more zinc fingers, each zinc finger having a recognition helix that binds to a target subsite in the selected sequence(s) (e.g., gene(s)). In certain embodiments, the repressor comprises a DNA-binding domain (ZFP) operably linked to a transcriptional repression domain to create an non-naturally occurring transcription factor (ZFP-TF repressor). Optionally, the ZFP-TF repressor comprises additional components, including but not limited to a nuclear localization signal (NLS). In some embodiments these non-naturally occurring TFs (e.g., ZFP-TFs) include protein interaction domains (or “dimerization domains”) that allow multimerization when bound to DNA.

In certain embodiments, the zinc finger proteins (ZFPs) as described herein can be placed in operative linkage with a regulatory domain (or functional domain) as part of a fusion protein. In some embodiments, the functional domain is, for example, a transcriptional activation domain, a transcriptional repression domain and/or a nuclease (cleavage) domain. By selecting either an activation domain or repression domain for use with the DNA-binding domain, such molecules are used either to activate or to repress gene expression. In some embodiments, the present invention provides a molecule comprising a ZFP targeted to a mHITT as described herein fused to a transcriptional repression domain is used to down-regulate mutant HTT expression. In some embodiments, a fusion protein comprising a ZFP targeted to a wild-type HTT allele fused to a transcription activation domain that can up-regulate the wild type HTT allele is provided. In certain embodiments, the activity of the regulatory domain is regulated by an exogenous small molecule or ligand such that interaction with the cell's transcription machinery will not take place in the absence of the exogenous ligand, while in other embodiments, the exogenous small molecule or ligand prevents the interaction. Such external ligands control the degree of interaction of the ZFP-TF with the transcription machinery. The regulatory domain(s) may be operatively linked to any portion(s) of one or more of the ZFPs, including between one or more ZFPs, exterior to one or more ZFPs and any combination thereof. Any of the fusion proteins described herein may be formulated into a pharmaceutical composition.

In yet another aspect, a polynucleotide encoding one or more of the DNA binding proteins and/or fusion molecules (e.g., non-naturally occurring transcription factors) as described herein is provided. In certain embodiments, the polynucleotide is carried on a viral (e.g., AAV or Ad or HSV-1, or VLP, Sheridan, Nature Biotechnology, 40, pages 809-811 (2022); Gurevich, Nature Medicine, 28, pages 780-788 (2022)) vector and/or a non-viral means (e.g., plasmid or mRNA vector or aptamer). Non-limiting examples of non-viral means include vectors, liposomes, nanoparticles, other lipid containing complexes including lipid nanoparticles (LNPs), other macromolecular complexes, inorganic nanoparticles, synthetic modified mRNA, unmodified mRNA, small molecules, non-biologically active molecules (e.g., gold particles), polymerized molecules (e.g., dendrimers), naked DNA, phages, transposons, episomes, plasmid vectors, bacteriophage vectors, cosmids, phagemids, artificial chromosomes, and the like. Host cells comprising these polynucleotides (e.g., rAAV vectors) or non-viral means and/or pharmaceutical compositions comprising the polynucleotides, proteins and/or host cells as described herein are also provided. In certain embodiments, the polynucleotide comprises at least one sequence as shown in Table 3. Compositions comprising one or more of these polynucleotides are also provided.

In some embodiments, the polynucleotide encoding the DNA binding protein and/or non-naturally occurring transcription factor (e.g., ZFP-TF) is an mRNA. In some embodiments, the mRNA may be chemically modified (See e.g. Kormann et al. (2011) Nature Biotechnology 29 (2): 154-157). In other embodiments, the mRNA may comprise an ARCA cap (see U.S. Pat. Nos. 7,074,596 and 8,153,773). In further embodiments, the mRNA may comprise a mixture of unmodified and modified nucleotides (see U.S. Patent Publication No. 2012/0195936).

In yet another aspect, a gene delivery vector comprising one or more of the polynucleotides described herein is provided. In certain embodiments, the vector is an adenovirus vector (e.g., an Ad5/F35 vector), a lentiviral vector (LV) including integration competent or integration-defective lentiviral vectors, an AAV vector (AAV), also referred to as a recombinant adeno-associated viral vector (rAAV), HSV-1 or a VLP, e.g., vector pseudotyped with VSV-G or other envelope proteins or a hybrid vector comprised of different AAV elements with those of mammalian bocaviruses (BoVs) and prokaryotic bacteriophages. (Fakhiri, 2021, Molecular Therapy, 29 (12): 3359-82.)

A blood-brain barrier (BBB) exists between cerebrovascular and cerebral cells to block transport and exchange of substances. Due to BBB, pharmaceutical compositions need to be administered directly into brain, which remains challenging and involves risks for patients. In certain embodiments, the AAV vector is an AAV1, AAV2, AAV5, AAV7, AAV9, or AAVrh10 or other BBB-penetrating AAV vector (e.g., as described in PCT publications WO2022221400A2, WO2023091948A1 and WO2020014471A1, incorporated herein by reference in entirety). Exemplary BBB-penetrating AAV vectors include, but are not limited to, VCAP-101, VCAP-102, 9P801, VCAP-100, VCAP-103, PAL1A, PAL1B, PAL1C, PAL2, CereAAV, Dyno bCAP1, AAV.CAP-B10, AAV.CAP-B20, AAV2-BRIN, AAV2-BR1, STAC-BBBR, or AAV-TT, or AAV-BI-hTFR1 among others (Stanton et al., Cell Press Med 4, 31-50; Goertsen et al., Nat. Neuroscience, 2022, 25 (1): 106-115; Tordo et al 2018, Brain. 2018, 141 (7): 2014-2031).

The AAV vector comprise one or more of the ZFP-TF polynucleotides shown in Table 3 (any one or more of SEQ ID NO: 10-29).

Additionally, pharmaceutical compositions comprising the nucleic acids and/or proteins e.g., ZFPs and/or fusion molecules (e.g., non-naturally occurring transcription factors comprising the ZFPs) are also provided. For example, certain compositions include a nucleic acid comprising a sequence that encodes one of the ZFPs described herein operably linked to a regulatory sequence, combined with a pharmaceutically acceptable carrier or diluent, wherein the regulatory sequence allows for expression of the nucleic acid in a cell. In certain embodiments, the ZFPs encoded are specific for a HD HTT allele. In some embodiments, pharmaceutical compositions comprise ZFPs that modulate a HD mHTT allele and ZFPs that modulate a neurotrophic factor. Proteins encoded by the gene therapy constructs disclosed herein include one of more ZFPs and a pharmaceutically acceptable carrier or diluent.

In certain embodiments, the pharmaceutical compositions comprise one or more polynucleotides of Table 3 for repression of HTT. In certain embodiments, pharmaceutical compositions comprising AAV vectors described herein comprise between 1×10⁸ and 5×10¹⁶ vg (or any value therebetween), even more preferably between 1×10⁸ and 1×10¹⁶ vg (or any value therebetween), even more preferably between 1×10¹³ and 5×10¹⁵ vg (or any value therebetween) of AAV-ZFP-TFs. In certain embodiments, AAV vectors are administered at a dose of between 1×10¹¹ and 1×10¹⁵ (or any value therebetween) vg per striatum, cortex, caudate, putamen, thalamus, or globus pallidus region of the brain, for example, including but not limited to 1e8, 1e9, 1e10, 1e11, 1e12, 1e13, 1e14 or 1e15 vg per striatum, cortex, caudate, putamen, thalamus, globus pallidus region of the brain). In some embodiments, the administration is at a dose of between 1×10¹¹ vg/kg and 1×10¹⁵ vg/kg by intravenous injection. In some embodiments, the administration is at a dose of between 1×10¹² vg/kg and 1×10¹⁴ vg/kg by intravenous administration. In some embodiments, the administration is at a dose of between about 5e13×5e15 vg by intravenous injection (for example, based on 70 kg body weight).

Intra-striatal administration may be to a single hemisphere or, preferably, bilaterally (at the same or different doses). In some embodiments, delivery is through non-viral means, e.g. lipid nanoparticle, liposome. Also provided is an isolated cell comprising any of the proteins, polynucleotides and/or compositions as described herein.

In another aspect, described herein are methods of modifying expression of an HTT gene in a cell (e.g., neuronal cell in vitro or in vivo in a brain of a subject, e.g., in one or more of the striatum, cortex, caudate, putamen, thalamus, or globus pallidus region of the brain), the method comprising administering to the cell one or more gene therapy constructs comprising ZFP-TF, pharmaceutical compositions and/or cells as described herein. In some embodiments, administration (e.g., of pharmaceutical compositions comprising AAV ZFP-TFs as described herein) is before and/or after the onset of disease symptoms at any dosage (e.g., between 1×10⁷ and 5×10¹⁵ AAV vg (or any value therebetween)). In some embodiments, administration is one-time or repeated at any intervals and repeated administrations may be at the same or different dosages. In some embodiments, the HTT gene comprises at least one wild-type and/or mutant HTT allele. In certain embodiments, HTT expression is repressed, for example where mutant HTT (mHTT) expression is preferentially repressed as compared to wild-type expression. Repression or HTT, including selective repression of mHTT, may persist days, weeks, months or years after one or more administrations of ZFP-TFs as described herein. In certain embodiments, selective repression of mHTT (as compared to wild type HTT) persists 6 months or more after a single administration.

In another aspect, provided herein are methods for treating and/or preventing Huntington's Disease using the methods and compositions (proteins, polynucleotides and/or cells) described herein. In some embodiments, the methods involve compositions where the polynucleotides and/or proteins may be delivered using a viral vector, including virus-like particles (VLPs), a non-viral vector and/or combinations thereof. In some embodiments, the viral vector is AAV, for example, a BBB-penetrant AAV. In some embodiments, non-viral delivery is using a lipid nanoparticle (LNP). Pharmaceutical compositions may also be delivered using standard techniques to the subject. In some embodiments, the methods involve compositions comprising stem cell populations comprising a ZFP, or altered with the ZFNs of the invention. The subject may comprise at least one mutant and/or wild-type HTT allele.

In a still further aspect, described here is a method of delivering one or more repressors of HTT (e.g., mHTT) to the brain of the subject using an rAAV (e.g., AAV9 or other AAV serotypes, for example, AAV1, AAV2, AAV5, AAV7, AAV9 or AAVrh10; for example, AAV comprising a capsid that penetrates a blood brain barrier) vector. In some embodiments, the repressor is delivered using AAV9. In some embodiments, the repressor is delivered using AAV5. In some embodiments, the repressor is delivered using AAV that penetrates the blood brain barrier, i.e., BBB penetrant-AAV. Exemplary BBB penetrant AAV include, but are not limited to, VCAP-101, VCAP-102, 9P801, VCAP-100, VCAP-103, PAL1A, PAL1B, PAL1C, PAL2, CereAAV, Dyno bCAP1, AAV.CAP-B10, AAV.CAP-B20, AAV2-BRIN, AAV2-BR1, STAC-BBBR, or AAV-TT, or AAV-BI-hTFR1, among others (e.g., as described in PCT publications WO2022221400A2, WO2023091948A1 and WO2020014471A1, incorporated herein by reference in entirety; Stanton et al., Cell Press Med 4, 31-50; Goertsen et al., Nat. Neuroscience, 2022, 25 (1): 106-115).

In some embodiments, the method of delivering one or more repressors of HTT to the brain, for example, the ZFP-TFs disclosed herein, is through non-viral means, e.g., a lipid nanoparticle or liposome.

Delivery may be to any brain region, for example, to one or more of the striatum, cortex, caudate, putamen, thalamus, or globus pallidus region of the brain (e.g., putamen; intrastriatal injection including stereotactic striatal injections) by any suitable means including via the use of a cannula (for example intracranial injection). Administration into the brain (e.g., striatum, cortex, caudate, putamen, thalamus, or globus pallidus region of the brain) may be to a single hemisphere or may be bilateral (e.g., at the same or different doses when bilateral). In some embodiments, delivery is through direct injection into the intrathecal space. In some embodiments, delivery is through intracerebroventricular (ICV) injection. In some embodiments, delivery is through intracerebral (ICM) microfusion. In some embodiments, delivery is through intrathecal injection. In further embodiments, delivery in through intravenous injection. The rAAV vector provides widespread delivery of the repressor to brain of the subject, including via anterograde and retrograde axonal transport to brain regions not directly administered the vector (e.g., delivery to the striatum) results in delivery to other structures such as the forebrain, hindbrain cortex, substantia nigra, thalamus, etc. In certain embodiments, one or more gene therapy constructs comprising ZFP-TF (or pharmaceutical compositions comprising the gene therapy constructs comprising ZFP-TF) of Table 3 are delivered to the subject. Any one or combination of repressors shown in Table 3 may be used (e.g., 1, 2, 3, 4 or 5 repressors in any combinations).

Thus, in other aspects, described herein is a method of preventing and/or treating HD in a subject, the method comprising administering at least one repressor of a mutant HTT (mHTT) allele to the subject. The repressor may be administered in polynucleotide form, for example using a viral (e.g., AAV) and/or non-viral vector (e.g., plasmid and/or mRNA) in protein form and/or via a pharmaceutical composition as described herein (e.g., pharmaceutical compositions comprising one or more polynucleotide, one or more AAV vectors, one or more LNP compositions, one or more fusion molecules and/or one or more cells as described herein). In certain embodiments, the repressor is administered to the CNS (e.g., striatum or other regions) of the subject. The repressor may provide therapeutic benefits, including, but not limited to, reducing mHTT RNA in blood and/or CSF, reducing mHTT protein levels in blood and/or CSF, reducing the formation of mHTT aggregates in HD neurons of a subject with HD (including reducing mHTT aggregation without effecting nuclear aggregation); reducing cell death in a neuron or population of neurons (e.g., an HD neuron or population of HD neurons); and/or reducing motor deficits (e.g., clasping, chorea, balance issues etc.) in HD subjects, improvement of the total motor score (TMS), improvement of Composite Unified Huntington's Disease Rating Scale (cUHDRS), improvement of Total Functional Capacity (TFC). In certain embodiments, mutant HTT expression is repressed by administration to the subject one or more proteins and/or polynucleotides (or pharmaceutical compositions comprising these proteins and/or polynucleotides) of Table 3 are delivered to the subject.

In any of the methods described herein, the repressor of the mutant HTT allele may be a ZFP-TF, for example a fusion protein comprising a ZFP that binds specifically to a mutant HTT allele and a transcriptional repression domain (e.g., KOX, KRAB, etc.). In certain embodiments, the ZFP-TF comprises a ZFP having the recognition helix regions of the ZFPs shown in Table 1, including the ZFP-TF repressors encoded by polynucleotides as shown in Table 3. In any of the methods described herein, the repressor(s) may be delivered to the subject (e.g., brain) as a protein, polynucleotide or any combination of protein and polynucleotide. In certain embodiments, the repressor(s) is (are) delivered using an AAV (e.g., AAV5, AAV9 or a BBB-penetrant AAV) vector. In some embodiments, the repressor is a fusion protein. In other embodiments, repressor is delivered in RNA form. In other embodiments, the repressor(s) is (are) delivered using a combination of any of the expression constructs described herein, for example one repressor (or portion thereof) on one expression construct (e.g., AAV such as AAV5, AAV9, among others) and one repressor (or portion thereof) on a separate expression construct (rAAV or other viral or non-viral construct).

Furthermore, in any of the methods described herein, the repressors can be delivered at any concentration (dose) that provides the desired effect. As shown herein, HTT repression can be achieved in vivo with exposure as low as 1 VG/cell in the subject. In preferred embodiments, the repressor is delivered using a recombinant adeno-associated virus vector at 10,000-500,000 vector genome/cell (or any value therebetween). In other embodiments, the repressor is delivered using a plasmid construct at 150-1,500 ng/100,000 cells (or any value therebetween). In other embodiments, the repressor is delivered as mRNA at 0.003-1,500 ng/100,000 cells (or any value therebetween). In some embodiments, the AAV dose is calculated per subject. For example, AAV vectors as described herein can comprise between 1×10⁷ and 5×10¹⁶ vg (or any value therebetween), even more preferably between 1×10⁹ and 1×10¹⁵ vg (or any value therebetween), even more preferably between 1×10¹² and 1×10¹⁴ vg (or any value therebetween). In certain embodiments, AAV vectors are administered at a dose of between 1×10¹² and 1×10¹⁵ (or any value therebetween) vg. In some embodiments, the administration is at a dose of between 1×10¹¹ vg/kg and 1×10¹⁴ vg/kg, between 1×10¹² vg/kg and 1×10¹³ vg/kg by intravenous administration. In some embodiments, the administration is at a dose of between about 1e15 and 5e15 vg by intravenous injection.

Intra-striatal administration may be to a single hemisphere or, preferably, bilaterally (at the same or different doses). For example, in some embodiments, the repressor is delivered at approximately 9e13 vg, or between approximately 9e10 vg and 5e14 vg, or between approximately 3e11 vg and 9e13 vg. In some embodiments, the AAV dose is less than 9e10 vg (for example 6e8 vg or less), and in other embodiments, the AAV dose is greater that 9e13 vg.

In any of the methods described herein, the compositions and methods described herein can yield about 70% or greater, about 75% or greater, about 85% or greater, about 90% or greater, about 92% or greater, or about 95% or greater repression of the mutant HTT allele expression in one or more HD neurons of the subject. Furthermore, the compositions and methods described herein can exhibit selectivity for HTT (e.g., mHTT) repression (as compared to repression of off-target sites) by at least 50%, for example, 50%-95%, 60%-80% (or any value therebetween), or greater than 85% as compared to the control.

In further aspects, the invention described herein comprises one or more HTT-modulating transcription factors, such as an HTT-modulating transcription factors comprising one or more of a zinc finger protein (ZFP TFs). In certain embodiments, the HTT-modulating transcription factor can repress expression of a mutant HTT allele in one or more HD neurons of a subject. The repression can be about 50% or greater, 55% or greater, 60% or greater, 65% or greater, 70% or greater, 75% or greater, 80% or greater, 85% or greater, 90% or greater, 92% or greater, or 95% or greater repression of the mutant HTT alleles in the one or more HD neurons of the subject as compared to untreated (e.g., wild-type) neurons of the subject. In certain embodiments, the HTT-modulating transcription factor can be used to achieve one or more of the methods described herein. In certain embodiments, the ZFP-TF comprises an amino acid sequence of a mHTT repressor as shown in Table 3.

In some embodiments, therapeutic efficacy is measured using the Unified Huntington's Disease Rating Scale (UHDRS) (Huntington Study Group (1996) Mov Disord 11 (2): 136-142) for analysis of overt clinical symptoms. In other embodiments, efficacy in patients is measured using Positron Emission Tomography (PET) and Magnetic Resonance Imaging (MRI) imaging. In some embodiments, treatment with the mutant HTT modulating transcription factor prevents any further development of overt clinical symptoms and prevents any further loss of neuron functionality. In other embodiments, treatment with the mutant HTT modulating transcription factor improves clinical symptoms (e.g., motor function as determined using known measures such as clasping behavior, rotating rod analysis and the like) and improves neuron function.

Also provided is a kit comprising one or more of the HTT-modulators (e.g., repressors) and/or polynucleotides comprising components of and/or encoding the HTT-modulators (or components thereof) as described herein. The kits may further comprise cells (e.g., neurons), reagents (e.g., for detecting and/or quantifying mHTT protein, for example in CSF) and/or instructions for use, including the methods as described herein.

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. Molecular Cloning: A Laboratory Manual, Second edition, Cold Spring Harbor Laboratory Press, 1989 and Third edition, 2001; Ausubel et al., Current Protocols in Molecular Biology, John Wiley & Sons, New York, 1987 and periodic updates; the series Methods in Enzymology, Academic Press, San Diego; Wolffe, Chromatin Structure and Function, Third edition, Academic Press, San Diego, 1998; Methods in Enzymology, Vol. 304, “Chromatin” (P. M. Wassarman and A. P. Wolffe, eds.), Academic Press, San Diego, 1999; and Methods in Molecular Biology, Vol. 119, “Chromatin Protocols” (P. B. Becker, ed.) Humana Press, Totowa, 1999.

### Definitions

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acid.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

Zinc finger binding domains can be “engineered” to bind to a predetermined nucleotide sequence, for example via engineering (altering one or more amino acids) of the recognition helix region of a naturally occurring zinc finger protein. Therefore, engineered zinc finger proteins are proteins that are non-naturally occurring. Non-limiting examples of methods for engineering zinc finger proteins are design and selection. A “designed” zinc finger protein is a protein not occurring in nature whose design/composition results principally from rational criteria. Rational criteria for design include application of substitution rules and computerized algorithms for processing information in a database storing information of existing ZFP designs and binding data. A “selected” zinc finger protein is a protein not found in nature whose production results primarily from an empirical process such as phage display, interaction trap or hybrid selection. See, for example, U.S. Pat. Nos. 8,586,526; 6,140,081; 6,453,242; 6,746,838; 7,241,573; 6,866,997; 7,241,574 and 6,534,261; see also International Patent Publication No. WO 03/016496.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid. The term also includes systems in which a polynucleotide component associates with a polypeptide component to form a functional molecule.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “multimerization domain”, (also referred to as a “dimerization domain” or “protein interaction domain”) is a domain incorporated at the amino, carboxy or amino and carboxy terminal regions of a ZFP TF. These domains allow for multimerization of multiple ZFP TF units such that larger tracts of trinucleotide repeat domains become preferentially bound by multimerized ZFP TFs relative to shorter tracts with wild-type numbers of lengths. Examples of multimerization domains include leucine zippers. Multimerization domains may also be regulated by small molecules wherein the multimerization domain assumes a proper conformation to allow for interaction with another multimerization domain only in the presence of a small molecule or external ligand. In this way, exogenous ligands can be used to regulate the activity of these domains.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., T-cells).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP is fused to an activation domain, the ZFP and the activation domain are in operative linkage if, in the fusion polypeptide, the ZFP is able to bind its target site and/or its binding site, while the activation domain is able to upregulate gene expression. ZFPs fused to domains capable of regulating gene expression are collectively referred to as “ZFP-TFs” or “zinc finger transcription factors.”

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and International Patent Publication No. WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles. The term includes viral and non-viral vectors, including but not limited to plasmid, mRNA, AAV (also referred to herein as “recombinant AAV” or “rAAV”), adenovirus vectors (Ad), lentiviral vectors (e.g., IDLV), lipid nanoparticles, liposomes and the like.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest.

### DNA-Binding Domains

The methods described herein make use of compositions, for example HTT-modulating transcription factors, comprising a DNA-binding domain that specifically binds to a target sequence in an HTT gene, particularly that bind to a mutant HTT allele (mHTT) comprising a plurality of trinucleotide repeats. Any polynucleotide or polypeptide DNA-binding domain can be used in the compositions and methods disclosed herein, for example DNA-binding proteins (e.g., ZFPs) or DNA-binding polynucleotides (e.g., single guide RNAs). In certain embodiments, the DNA-binding domain binds to a target site comprising 9 to 28 (or any value therebetween including 10, 11, 12, 13, 14, 15, 16, 17, 18, 19, 20, 21, 22, 23, 24, 25, 26 or 27) contiguous copies of nucleotides of SEQ ID NO: 6.

In certain embodiments, the mHTT-modulating transcription factor, or DNA binding domain therein, comprises a zinc finger protein. Selection of target sites; ZFPs and methods for design and construction of fusion proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453 and 6,200,759; and International Patent Publication Nos. WO 95/19431; WO 96/06166; WO 98/53057; WO 98/54311; WO 00/27878; WO 01/60970 WO 01/88197; WO 02/099084; WO 98/53058; WO 98/53059; WO 98/53060; WO 02/016536; and WO 03/016496.

In certain embodiments, the ZFPs can bind selectively to either a mutant HTT allele or a wild-type HTT sequence. HTT target sites typically include at least one zinc finger but can include a plurality of zinc fingers (e.g., 2, 3, 4, 5, 6 or more fingers). See, e.g., U.S. Pat. Nos. 9,234,016; 9,943,565; 8,841,260; 9,499,597; and U.S. Patent Publication Nos. 2015/0335708; 2018/0200332; 2017/0096460; 2017/0035839; 2016/0296605; and 2019/0322711. Usually, the ZFPs include at least three fingers. Certain of the ZFPs include four, five or six fingers, while some ZFPs include 7, 8, 9, 10, 11 or 12 fingers. The ZFPs that include three fingers typically recognize a target site that includes 9 or 10 nucleotides; ZFPs that include four fingers typically recognize a target site that includes 12 to 14 nucleotides; while ZFPs having six fingers can recognize target sites that include 18 to 21 nucleotides. The ZFPs can also be fusion proteins that include one or more regulatory domains, which domains can be transcriptional activation or repression domains. In some embodiments, the fusion protein comprises two ZFP DNA binding domains linked together. These zinc finger proteins can thus comprise 8, 9, 10, 11, 12 or more fingers. In some embodiments, the two DNA binding domains are linked via an extendable flexible linker such that one DNA binding domain comprises 4, 5, or 6 zinc fingers and the second DNA binding domain comprises an additional 4, 5, or 5 zinc fingers. In some embodiments, the linker is a standard inter-finger linker such that the finger array comprises one DNA binding domain comprising 8, 9, 10, 11 or 12 or more fingers. In other embodiments, the linker is an atypical linker such as a flexible linker. The DNA binding domains are fused to at least one regulatory domain and can be thought of as a ‘ZFP-ZFP-TF’ architecture. Specific examples of these embodiments can be referred to as “ZFP-ZFP-KOX” which comprises two DNA binding domains linked with a flexible linker and fused to a KOX repressor and “ZFP-KOX-ZFP-KOX” where two ZFP-KOX fusion proteins are fused together via a linker.

Alternatively, the DNA-binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22:1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 2007/0117128.

“Two handed” zinc finger proteins are those proteins in which two clusters of zinc finger DNA binding domains are separated by intervening amino acids so that the two zinc finger domains bind to two discontinuous target sites. An example of a two handed type of zinc finger binding protein is SIP1, where a cluster of four zinc fingers is located at the amino terminus of the protein and a cluster of three fingers is located at the carboxyl terminus (see Remacle et al. (1999) EMBO Journal 18 (18): 5073-5084). Each cluster of zinc fingers in these proteins is able to bind to a unique target sequence and the spacing between the two target sequences can comprise many nucleotides. Two-handed ZFPs may include a functional domain, for example fused to one or both of the ZFPs. Thus, it will be apparent that the functional domain may be attached to the exterior of one or both ZFPs or may be positioned between the ZFPs (attached to both ZFPs).

Specific examples of HTT-targeted ZFPs are disclosed in Table 1 as well as in U.S. Pat. Nos. 9,234,016; 8,841,260; and 6,534,261; U.S. Patent Publication Nos. 2017/0096460; 2015/0056705; 2015/0335708; and 2019/0322711, which are incorporated by reference for all purposes in its entirety herein. The first column in this table is an internal reference name (number) for a ZFP and corresponds to the same name in column 1 of Table 2. “F” refers to the finger and the number following “F” refers which zinc finger (e.g., “F1” refers to finger 1).

TABLE 1
HTT-targeted zinc finger proteins

| SBS | Design | | | | | |
|---|---|---|---|---|---|---|
| # | F1 | F2 | F3 | F4 | F5 | F6 |
| 46025 | CPSHLTR | QSGDLTR | KHGNLSE | KRCNLRC | RQFNRHQ | |
| | (SEQ ID | (SEQ ID | (SEQ ID | (SEQ ID | (SEQ ID | |
| | NO: 4) | NO: 1) | NO: 2) | NO: 3) | NO: 5) | |
| 45723 | SPEQLSR | QWSTRKR | KQGNLVE | KRCNLRC | N/A | |
| | (SEQ ID | (SEQ ID | (SEQ ID | (SEQ ID | | |
| | NO: 7) | NO: 8) | NO: 9) | NO: 3) | | |

The sequence and location for the target sites of these proteins are disclosed in Table 2. Nucleotides in the target site that are contacted by the ZFP recognition helices are indicated in uppercase letters; non-contacted nucleotides indicated in lowercase.

TABLE 2
Target sites on human and mouse HTT

| SBS # | Target Site |
|---|---|
| 46025 | agCAGCAGCAGcaGCAGCAgcagcagca (SEQ ID NO: 6) |
| 45723 | agCAGCAGcaGCAGCAgcagcagcagca (SEQ ID NO: 6) |

ZFP-TFs as described herein may also include one or more mutations outside recognition helix regions (e.g., to the backbone regions), including mutations as described in U.S. Patent Publication No. 2018/0087072.

Any suitable promoter can be used to drive expression of the ZFP-TF. In preferred embodiments, a phosphoglycerate kinase 1 (PGK) promoter is used. In other preferred embodiments, a ubiquitin C (UBC) promoter is used. The PGK and UBC promoters, for example, provide an optimal expression profile for the ZFP-TF, preventing toxicity, adverse immune reaction or silencing due to overexpression. For ubiquitous expression, other promoters that can be used include cytomegalovirus (CMV), Rous Sarcoma Virus (RSV), CAG promoter, chicken beta actin (CBh), human beta actin, mammalian elongation factor 1α (EF1alpha), EFS, Simian Virus 40 (SV40), ferritin heavy or light chains, HSP90AB1, etc. For brain or other CNS cell expression, suitable promoters can include: SynapsinI for all neurons, CaMKIIalpha for excitatory neurons, GAD67 or GAD65 or VGAT for GABAergic neurons, etc. For endothelial cells, suitable promoters can include ICAM. For hematopoietic cells suitable promoters can include IFNbeta or CD45. For muscle tissue, suitable promoters can include Human skeletal α-actin, muscle creatine kinase (MCK/CKM, creatine kinase, M-type), CK6, MHCK7, desmin promoter, MLC, myosin heavy chain gene (αMHC) promoter, myosin light-chain promoter (MLC2v), cardiac troponin T promoter (cTnT), among others.

In some embodiments, the promoters are flanked by an AAV ITR. This can be advantageous for eliminating the need for an additional promoter element, which can take up space in the vector. The additional space freed up can be used to drive the expression of additional elements, such as a guide nucleic acid or a selectable marker. ITR activity is relatively weak, so it can be used to reduce potential toxicity due to overexpression.

### Fusion Molecules

The DNA-binding domains may be fused to any additional molecules (e.g., polypeptides) for use in the methods described herein. In certain embodiments, the methods employ fusion molecules comprising at least one DNA-binding molecule (e.g., ZFP) and a heterologous regulatory (functional) domain (or functional fragment thereof).

In certain embodiments, the functional domain comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members, etc.); DNA repair enzymes and their associated factors and modifiers; DNA rearrangement enzymes and their associated factors and modifiers; chromatin associated proteins and their modifiers (e.g., kinases, acetylases and deacetylases); and DNA modifying enzymes (e.g., methyltransferases, topoisomerases, helicases, ligases, kinases, phosphatases, polymerases, endonucleases) and their associated factors and modifiers. See, e.g., U.S. Patent Publication No. 2013/0253040, incorporated by reference in its entirety herein.

Suitable domains for achieving activation include the HSV VP16 activation domain (see, e.g., Hagmann et al. (1997) J. Virol. 71:5952-5962) nuclear hormone receptors (see, e.g., Torchia et al. (1998) Curr. Opin. Cell. Biol. 10:373-383); the p65 subunit of nuclear factor kappa B (Bitko & Barik (1998) J. Virol. 72:5610-5618 and Doyle & Hunt (1997) Neuroreport 8:2937-2942; Liu et al. (1998) Cancer Gene Ther. 5:3-28), or artificial chimeric functional domains such as VP64 (Beerli et al. (1998) Proc. Natl. Acad. Sci. USA 95:14623-33), and degron (Molinari et al. (1999) EMBO J. 18:6439-6447). Additional exemplary activation domains include, Oct 1, Oct-2A, Sp1, AP-2, and CTF1 (Seipel et al. (1992) EMBO J. 11:4961-4968) as well as p300, CBP, PCAF, SRC1 PvALF, AtHD2A and ERF-2. See, for example, Robyr et al. (2000) Mol. Endocrinol. 14:329-347; Collingwood et al. (1999) J. Mol. Endocrinol. 23:255-275; Leo et al. (2000) Gene 245:1-11; Manteuffel-Cymborowska (1999) Acta Biochim. Pol. 46:77-89; Mckenna et al. (1999) J. Steroid Biochem. Mol. Biol. 69:3-12; Malik et al. (2000) Trends Biochem. Sci. 25:277-283; and Lemon et al. (1999) Curr. Opin. Genet. Dev. 9:499-504. Additional exemplary activation domains include, but are not limited to, OsGAI, HALF-1, C1, AP1, ARF-5, -6, -7, and -8, CPRF1, CPRF4, MYC-RP/GP, and TRAB1. See, for example, Ogawa et al. (2000) Gene 245:21-29; Okanami et al. (1996) Genes Cells 1:87-99; Goff et al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haussels et al. (2000) Plant J. 22:1-8; Gong et al. (1999) Plant Mol. Biol. 41:33-44; and Hobo et al. (1999) Proc. Natl. Acad. Sci. USA 96:15,348-15,353.

Exemplary repression domains include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B), Rb, and MeCP2. See, for example, Bird et al. (1999) Cell 99:451-454; Tyler et al. (1999) Cell 99:443-446; Knoepfler et al. (1999) Cell 99:447-450; and Robertson et al. (2000) Nature Genet. 25:338-342. Additional exemplary repression domains include, but are not limited to, ROM2 and AtHD2A. See, for example, Chem et al. (1996) Plant Cell 8:305-321; and Wu et al. (2000) Plant J. 22:19-27.

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al. (2000) Proc. Natl. Acad. Sci. USA 97:3930-3935.

The fusion molecule may be formulated with a pharmaceutically acceptable carrier, as is known to those of skill in the art. See, for example, Remington's Pharmaceutical Sciences, 17th ed., 1985; and co-owned International Patent Publication No. WO 00/42219.

The functional component/domain of a fusion molecule can be selected from any of a variety of different components capable of influencing transcription of a gene once the fusion molecule binds to a target sequence via its DNA binding domain. Hence, the functional component can include, but is not limited to, various transcription factor domains, such as activators, repressors, co-activators, co-repressors, and silencers.

In certain embodiments, the fusion molecule comprises one or more ZFP-TFs (repressors) in which the ZFP is operably linked to a transcriptional repression domain. Non-limiting examples of repression domains include KOX (KRAB) domains and the like. Additional elements may also be included, for example an NLS and any linkers may be used between the zinc finger domains and/or between the ZFP and the repression domain (and/or any additional elements). Polynucleotides encoding these ZFP-TF repressors may also include further additional elements such as a promoter driving expression of the ZFP-TF, enhancers, insulators, and the like.

Table 3 shows the polynucleotide sequence exemplary ZFP-TFs comprising the ZFPs described herein (identified by name in the first column).

TABLE 3
Nucleotide Sequence of ZFP-TFs

| SEQ | | |
|---|---|---|
| ID | Variant | |
| NO | (# of CpGs) | Sequence |
| 10 | ZFP46025_P | ATGGCCCCCAAGAAAAAGCGGAAAGTGGGCATCCACGGGGTACCCGCCG |
| | (38 CpGs) | CTATGGCTGAGAGGCCCTTCCAGTGTCGAATCTGCATGCGTAACTTCAG |
| | | TTGTCCGTCCCACCTGACCCGCCACATCCGCACCCACACCGGCGAGAAG |
| | | CCTTTTGCCTGTGACATTTGTGGGAGGAAATTTGCCCAGTCCGGCGACC |
| | | TGACCCGCCATACCAAGATACACACGCCTAATCCTCATCGCCGCACTGA |
| | | TCCCAGCCATAAGCCCTTCCAGTGTCGAATCTGCATGCGTAACTTCAGT |
| | | AAGCACGGCAACCTGTCCGAGCACATCCGCACCCACACCGGCGAGAAGC |
| | | CTTTTGCCTGTGACATTTGTGGGAGGAAATTTGCCAAGCGCTGTAACCT |
| | | GCGCTGTCATACCAAGATACACACGGGCTCCCAATCCCCCTTCCAGTGT |
| | | CGAATCTGCATGCGTAAGTTTGCCCGCCAGTTCAACCGCCACCAGCATA |
| | | CCAAGATACACCTGCGCCAAAAAGATGCGGCCCGGGGATCCGGCATGGA |
| | | TGCTAAGTCACTAACTGCCTGGTCCCGGACACTGGTGACCTTCAAGGAT |
| | | GTATTTGTGGACTTCACCAGGGAGGAGTGGAAGCTGCTGGACACTGCTC |
| | | AGCAGATCGTGTACAGAAATGTGATGCTGGAGAACTATAAGAACCTGGT |
| | | TTCCTTGGGTTATCAGCTTACTAAGCCAGATGTGATCCTCCGGTTGGAG |
| | | AAGGGAGAAGAGCCCTGGCTGGTGGAGAGAGAAATTCACCAAGAGACCC |
| | | ATCCTGATTCAGAGACTGCATTTGAAATCAAATCATCAGTTTAA |
| 11 | ZFP46025_co1 | ATGGCCCCTAAGAAGAAGAGAAAAGTTGGCATACATGGAGTCCCTGCTG |
| | (4 CpGs) | CAATGGCTGAAAGACCATTCCAGTGCAGAATCTGCATGAGGAACTTCTC |
| | | CTGCCCTTCCCACCTGACCAGGCACATCAGGACCCACACTGGAGAAAAG |
| | | CCCTTTGCCTGTGATATCTGTGGAAGAAAATTTGCCCAGTCAGGGGACC |
| | | TCACCAGACACACCAAAATTCACACCCCCAACCCCCATAGAAGAACTGA |
| | | CCCCTCCCATAAGCCCTTCCAGTGCCGGATCTGCATGCGGAACTTCAGC |
| | | AAGCATGGCAACCTGTCAGAGCACATTAGGACTCACACTGGGGAGAAGC |
| | | CCTTCGCCTGTGACATCTGTGGCCGCAAGTTTGCCAAGAGGTGCAACCT |
| | | GAGGTGCCATACCAAGATCCACACTGGATCCCAGTCCCCATTCCAATGC |
| | | AGAATTTGCATGAGAAAGTTTGCTAGACAATTCAACAGACACCAGCACA |
| | | CCAAGATTCACCTGAGGCAGAAGGATGCAGCCAGAGGTTCAGGAATGGA |
| | | TGCCAAGTCCCTCACTGCCTGGAGCAGAACCCTGGTCACCTTCAAGGAT |
| | | GTGTTTGTGGACTTCACCAGAGAAGAGTGGAAGCTCCTGGACACTGCCC |
| | | AGCAAATTGTGTACAGGAATGTGATGCTGGAGAACTACAAGAACCTGGT |
| | | GTCCCTGGGTTACCAGCTCACCAAGCCTGATGTCATCCTGAGGCTGGAA |
| | | AAGGGAGAGGAACCTTGGTTGGTGGAGAGGGAAATCCACCAGGAAACTC |
| | | ACCCTGACTCAGAAACTGCCTTTGAGATCAAGAGCTCTGTGTAA |
| 12 | ZFP46025_co2 | ATGGCCCCTAAGAAGAAGAGAAAAGTTGGCATACATGGAGTCCCTGCTG |
| | (4 CpGs) | CAATGGCTGAAAGACCCTTCCAATGCCGGATTTGCATGAGAAACTTCTC |
| | | CTGCCCCTCCCACCTGACCAGACACATTAGGACTCACACTGGGGAGAAG |
| | | CCTTTTGCCTGTGACATCTGTGGAAGGAAGTTTGCTCAGTCAGGGGACC |
| | | TCACCAGACATACCAAGATCCACACCCCCAACCCCCATAGAAGAACTGA |
| | | CCCCTCCCATAAGCCTTTCCAGTGCCGGATCTGCATGAGGAACTTCTCC |
| | | AAGCATGGAAACCTGTCAGAACACATTAGAACCCACACTGGAGAAAAGC |
| | | CCTTCGCCTGTGATATCTGTGGTAGAAAGTTTGCCAAGAGATGCAACCT |
| | | GAGGTGTCACACCAAAATCCACACTGGCAGCCAGAGCCCATTCCAATGC |
| | | AGAATCTGCATGCGCAAGTTTGCCAGACAGTTCAACAGGCACCAGCACA |
| | | CTAAGATCCACCTGAGACAGAAAGATGCAGCCAGGGGTTCAGGAATGGA |
| | | TGCCAAGTCCCTCACTGCCTGGTCCAGGACCCTGGTCACCTTCAAGGAT |
| | | GTCTTTGTGGACTTCACCAGAGAAGAGTGGAAGCTCTTGGACACTGCCC |
| | | AGCAAATTGTGTACAGGAATGTGATGCTGGAGAACTACAAGAACCTGGT |
| | | GTCCCTGGGCTACCAGCTCACCAAGCCTGATGTGATCCTGAGGCTGGAA |
| | | AAGGGAGAGGAACCCTGGCTTGTGGAGAGGGAAATTCACCAGGAAACCC |
| | | ACCCTGACTCTGAGACTGCCTTTGAGATCAAGAGCTCAGTGTAA |
| 13 | ZFP46025_co3 | ATGGCCCCTAAGAAGAAGAGAAAAGTTGGCATTCATGGAGTCCCTGCAG |
| | (6 CpGs) | CTATGGCAGAAAGACCATTCCAGTGCAGAATTTGCATGAGAAACTTCTC |
| | | CTGCCCTTCCCACCTGACCAGGCATATCAGGACCCACACTGGGGAGAAG |
| | | CCCTTTGCCTGTGATATCTGTGGTAGAAAGTTTGCCCAGTCAGGGGACC |
| | | TGACCAGACACACCAAAATTCACACCCCCAACCCCCATCGGAGAACTGA |
| | | CCCCTCCCATAAGCCCTTCCAGTGCCGGATCTGCATGAGGAACTTCAGC |
| | | AAGCATGGAAACCTCTCAGAGCACATCAGAACTCACACTGGAGAAAAGC |
| | | CCTTCGCCTGTGACATCTGTGGAAGGAAGTTTGCCAAGAGATGCAACCT |
| | | GAGATGCCACACTAAGATCCACACTGGTAGCCAGTCCCCCTTCCAATGC |
| | | CGCATCTGCATGCGCAAGTTTGCTAGACAGTTCAACAGGCACCAGCACA |
| | | CCAAGATCCACCTGAGGCAGAAAGATGCAGCCAGAGGCTCAGGAATGGA |
| | | TGCCAAGTCCCTCACTGCCTGGTCCCGGACCCTTGTGACCTTCAAGGAT |
| | | GTCTTTGTGGACTTCACCAGAGAAGAGTGGAAGCTCCTGGACACTGCTC |
| | | AGCAAATTGTGTACAGGAATGTCATGCTGGAGAACTACAAGAACCTGGT |
| | | GTCCCTGGGCTACCAACTCACCAAGCCTGATGTGATCCTGAGGCTGGAA |
| | | AAGGGAGAAGAACCTTGGTTGGTGGAGAGGGAAATTCACCAGGAAACCC |
| | | ACCCTGACTCAGAGACTGCCTTTGAGATCAAGAGCTCTGTGTAA |
| 14 | ZFP46025_co4 | ATGGCCCCTAAGAAGAAGAGAAAAGTTGGCATTCATGGAGTCCCTGCAG |
| | (5 CpGs) | CTATGGCAGAAAGACCCTTCCAATGCCGGATCTGCATGCGGAACTTCTC |
| | | CTGCCCCTCCCACCTGACCAGACACATCAGGACCCACACTGGAGAAAAG |
| | | CCCTTTGCTTGTGACATCTGTGGTCGGAAGTTTGCCCAGTCAGGAGACT |
| | | TGACCAGGCATACCAAGATTCACACCCCCAACCCCCATAGAAGAACTGA |
| | | CCCCTCCCACAAGCCCTTCCAGTGCCGCATCTGCATGAGAAACTTCAGC |
| | | AAGCATGGCAACCTGTCAGAACACATCAGAACTCATACTGGAGAGAAGC |
| | | CTTTTGCCTGTGACATTTGTGGAAGAAAGTTTGCCAAGAGATGCAACCT |
| | | GAGGTGCCACACCAAGATCCACACTGGTAGCCAGAGCCCATTCCAGTGC |
| | | AGAATTTGCATGAGGAAGTTTGCTAGGCAGTTCAACAGGCACCAGCACA |
| | | CCAAAATCCACCTGAGGCAGAAAGATGCAGCCAGGGGCTCTGGGATGGA |
| | | TGCCAAGTCCCTCACTGCCTGGTCCAGAACCCTTGTGACCTTCAAGGAT |
| | | GTGTTTGTGGACTTCACCAGAGAAGAGTGGAAGCTCCTGGACACTGCCC |
| | | AGCAAATTGTCTACAGAAATGTCATGCTGGAGAACTACAAGAACCTGGT |
| | | GTCCCTGGGATACCAGCTCACCAAGCCTGATGTGATCCTCCGCCTGGAA |
| | | AAGGGGGAAGAACCTTGGTTGGTGGAGAGGGAAATCCACCAAGAGACTC |
| | | ACCCTGACTCAGAGACTGCCTTTGAGATCAAGTCCTCAGTGTAA |
| 15 | ZFP46025_co5 | ATGGCCCCTAAGAAGAAGAGAAAAGTTGGTATACATGGAGTCCCTGCTG |
| | (0 CpGs) | CAATGGCTGAAAGACCCTTCCAGTGCAGAATTTGTATGAGAAACTTCTC |
| | | CTGCCCTTCCCACCTGACCAGACATATCAGGACTCACACTGGGGAAAAG |
| | | CCCTTTGCCTGTGACATCTGTGGTAGAAAGTTTGCTCAGTCAGGGGACC |
| | | TCACCAGACACACCAAGATCCACACCCCCAACCCCCATAGAAGAACTGA |
| | | CCCCTCCCATAAGCCATTCCAATGCAGAATTTGCATGAGGAACTTCTCC |
| | | AAGCATGGCAACCTGTCAGAACACATCAGGACCCACACTGGAGAGAAGC |
| | | CTTTTGCATGTGATATCTGTGGAAGGAAGTTTGCCAAGAGATGCAACTT |
| | | GAGATGCCACACTAAGATTCACACTGGCAGCCAGAGCCCATTCCAGTGC |
| | | AGGATCTGCATGAGAAAATTTGCCAGACAGTTCAACAGACACCAACACA |
| | | CCAAAATCCACCTGAGGCAGAAAGATGCAGCCAGGGGATCTGGAATGGA |
| | | TGCCAAGTCCCTCACTGCCTGGAGCAGAACCCTGGTCACCTTCAAGGAT |
| | | GTGTTTGTGGACTTCACCAGGGAAGAGTGGAAGCTCCTGGACACTGCCC |
| | | AGCAAATTGTGTACAGGAATGTGATGCTGGAGAACTACAAGAACCTGGT |
| | | GTCCCTGGGCTACCAGCTCACCAAGCCTGATGTCATCCTGAGGCTGGAA |
| | | AAGGGAGAGGAACCCTGGCTTGTGGAGAGAGAGATTCACCAGGAAACCC |
| | | ACCCTGACTCAGAAACTGCCTTTGAGATCAAGTCCTCAGTGTAA |
| 16 | ZFP46025_co6 | ATGGCCCCCAAGAAAAAGAGAAAAGTTGGCATTCATGGAGTCCCTGCTG |
| | (0 CpGs) | CAATGGCTGAAAGACCATTCCAATGCAGGATCTGCATGAGGAACTTCAG |
| | | CTGCCCCTCCCACCTGACCAGGCACATTAGGACTCACACTGGAGAAAAG |
| | | CCCTTTGCCTGTGACATCTGTGGCAGGAAGTTTGCCCAGTCAGGGGACC |
| | | TCACCAGACATACCAAGATCCACACCCCCAACCCTCATAGAAGAACTGA |
| | | CCCTTCCCACAAGCCATTCCAGTGCAGAATCTGCATGAGAAACTTCTCC |
| | | AAGCATGGAAACCTGTCAGAGCACATCAGGACCCACACTGGGGAAAAGC |
| | | CTTTTGCTTGTGACATTTGTGGTAGAAAATTTGCCAAGAGGTGCAACCT |
| | | GAGATGCCACACCAAGATTCACACTGGATCCCAAAGCCCCTTCCAGTGC |
| | | AGAATTTGCATGAGAAAGTTTGCCAGGCAGTTCAACAGGCACCAGCACA |
| | | CTAAGATCCACCTGAGACAGAAAGATGCAGCCAGAGGCTCAGGAATGGA |
| | | TGCCAAGAGCCTCACTGCCTGGTCCAGAACCCTTGTGACCTTCAAGGAT |
| | | GTCTTTGTGGACTTCACCAGAGAAGAGTGGAAGCTCCTGGACACTGCCC |
| | | AGCAAATTGTGTACAGGAATGTGATGCTGGAGAACTACAAGAACCTGGT |
| | | GTCACTGGGTTACCAGCTCACCAAGCCTGATGTCATCCTGAGACTGGAA |
| | | AAGGGAGAGGAACCCTGGTTGGTGGAGAGAGAGATCCATCAGGAAACCC |
| | | ACCCTGACTCAGAAACTGCCTTTGAGATCAAGTCCTCTGTGTAA |
| 17 | ZFP46025_co5a | ATGGCCCCTAAGAAGAAGAGAAAAGTTGGCATTCATGGAGTGCCAGCAG |
| | (0 CpGs) | CAATGGCTGAGAGGCCATTCCAGTGCAGAATCTGTATGAGGAACTTCTC |
| | | CTGCCCCTCCCACCTCACAAGACACATCAGGACCCATACTGGAGAGAAG |
| | | CCCTTTGCCTGTGACATCTGTGGTAGAAAGTTTGCCCAGTCAGGGGACC |
| | | TGACCAGACACACTAAGATTCACACCCCCAACCCACATAGGAGGACTGA |
| | | CCCCTCCCATAAGCCCTTCCAGTGTAGAATCTGCATGAGGAATTTCTCC |
| | | AAGCATGGCAACCTGTCAGAACACATCAGAACCCACACTGGGGAAAAGC |
| | | CTTTTGCTTGTGATATCTGTGGAAGGAAATTTGCCAAGAGGTGCAACCT |
| | | TAGATGCCATACCAAAATTCACACTGGAAGCCAGAGCCCCTTCCAATGT |
| | | AGAATTTGCATGAGAAAATTTGCCAGACAATTCAACAGACACCAGCACA |
| | | CCAAGATCCACCTGAGACAAAAGGATGCAGCCAGAGGCTCTGGAATGGA |
| | | TGCCAAGAGCCTCACTGCCTGGAGCAGGACCCTGGTCACCTTCAAGGAT |
| | | GTGTTTGTGGACTTCACCAGAGAAGAGTGGAAGCTGCTGGACACTGCCC |
| | | AGCAAATTGTGTACAGAAATGTCATGCTGGAGAACTACAAGAACTTGGT |
| | | GTCCCTGGGTTACCAGCTCACCAAGCCTGATGTGATCCTGAGGCTGGAA |
| | | AAGGGAGAGGAACCTTGGTTGGTGGAGAGGGAAATCCACCAGGAAACCC |
| | | ACCCTGACTCAGAAACTGCCTTTGAGATCAAGTCCAGTGTCTAA |
| 18 | ZFP46025_co5b | ATGGCCCCCAAGAAGAAAAGAAAAGTTGGCATTCATGGGGTGCCTGCTG |
| | (0 CpGs) | CAATGGCAGAAAGGCCATTCCAGTGCAGAATCTGCATGAGGAACTTCTC |
| | | CTGCCCCTCCCACCTGACCAGACACATTAGAACCCACACTGGGGAGAAG |
| | | CCCTTTGCCTGTGACATCTGTGGCAGAAAATTTGCCCAGTCTGGGGACC |
| | | TCACCAGACACACTAAGATTCACACCCCCAACCCACATAGAAGAACTGA |
| | | CCCCAGCCACAAGCCCTTCCAGTGTAGAATCTGTATGAGAAACTTCAGC |
| | | AAGCATGGAAACCTGTCAGAGCACATCAGGACCCATACTGGAGAGAAGC |
| | | CTTTTGCTTGTGATATCTGTGGAAGGAAGTTTGCCAAGAGGTGCAACCT |
| | | GAGATGCCACACCAAGATCCACACTGGTTCCCAATCCCCATTCCAATGC |
| | | AGAATTTGCATGAGAAAGTTTGCAAGACAGTTCAACAGGCACCAGCATA |
| | | CTAAGATCCACCTGAGGCAGAAAGATGCTGCCAGGGGTTCAGGAATGGA |
| | | TGCCAAGTCCCTCACTGCCTGGAGCAGAACCCTTGTGACCTTCAAGGAT |
| | | GTCTTTGTGGACTTCACAAGGGAAGAGTGGAAGCTCCTGGACACTGCCC |
| | | AGCAAATTGTGTACAGGAATGTCATGCTGGAGAACTACAAGAACCTTGT |
| | | GTCCCTGGGCTACCAGCTGACCAAGCCTGATGTGATCCTGAGACTGGAA |
| | | AAGGGAGAGGAACCTTGGTTGGTGGAGAGGGAAATCCACCAGGAAACCC |
| | | ACCCTGACTCAGAGACTGCCTTTGAAATCAAGTCCTCAGTGTAA |
| 19 | ZFP46025_co5c | ATGGCCCCCAAGAAAAAGAGAAAAGTGGGCATTCATGGAGTGCCTGCAG |
| | (0 CpGs) | CTATGGCTGAAAGGCCATTCCAGTGTAGAATCTGCATGAGAAACTTCTC |
| | | CTGCCCTTCCCACCTGACCAGACACATCAGGACCCACACTGGAGAGAAA |
| | | CCCTTTGCTTGTGACATTTGTGGTAGAAAGTTTGCACAGTCAGGGGACC |
| | | TCACCAGGCATACCAAGATCCACACCCCAAACCCCCATAGAAGAACTGA |
| | | CCCCTCCCATAAGCCCTTCCAGTGCAGAATCTGTATGAGGAACTTCAGC |
| | | AAGCATGGAAACCTCTCAGAGCATATCAGAACTCACACTGGGGAGAAGC |
| | | CCTTTGCCTGTGACATCTGTGGGAGGAAGTTTGCCAAGAGATGCAACCT |
| | | CAGATGCCACACTAAGATTCACACTGGCAGCCAGTCCCCATTCCAATGC |
| | | AGAATTTGCATGAGGAAATTTGCCAGACAATTCAATAGGCACCAGCACA |
| | | CCAAGATTCACCTGAGGCAGAAGGATGCTGCCAGAGGCTCAGGAATGGA |
| | | TGCCAAGTCCCTGACTGCCTGGTCCAGGACCCTGGTCACCTTCAAGGAT |
| | | GTGTTTGTGGACTTCACCAGAGAAGAGTGGAAGCTGCTGGACACAGCCC |
| | | AGCAAATTGTGTACAGGAATGTCATGCTGGAGAACTACAAGAACCTGGT |
| | | GTCCCTGGGCTACCAGCTTACCAAGCCTGATGTCATCCTGAGACTGGAA |
| | | AAGGGAGAGGAACCTTGGTTGGTGGAGAGGGAAATCCACCAGGAAACCC |
| | | ACCCTGACTCAGAAACTGCCTTTGAGATCAAGAGCTCTGTGTAA |
| 20 | ZFP46025_co6a | ATGGCCCCCAAGAAGAAGAGGAAAGTTGGCATTCATGGAGTGCCTGCTG |
| | (0 CpGs) | CAATGGCTGAGAGGCCATTCCAGTGCAGAATCTGTATGAGGAACTTCAG |
| | | CTGCCCCAGCCACCTGACCAGACACATCAGGACCCACACTGGAGAAAAG |
| | | CCCTTTGCCTGTGACATTTGTGGGAGAAAATTTGCCCAGTCAGGGGACC |
| | | TCACCAGGCACACCAAGATCCATACCCCCAACCCACATAGAAGAACTGA |
| | | CCCTTCCCACAAGCCCTTCCAATGTAGAATCTGCATGAGAAACTTCTCC |
| | | AAGCATGGAAACCTCTCAGAGCACATTAGAACCCATACTGGGGAGAAGC |
| | | CTTTTGCTTGTGACATCTGTGGTAGAAAGTTTGCCAAGAGGTGCAACCT |
| | | GAGATGCCACACTAAGATCCACACTGGTAGCCAAAGCCCATTCCAATGC |
| | | AGAATTTGCATGAGGAAGTTTGCAAGACAGTTCAACAGACACCAGCACA |
| | | CCAAAATCCACCTGAGGCAGAAAGATGCAGCCAGAGGCTCAGGAATGGA |
| | | TGCCAAGTCCTTGACAGCCTGGTCCAGGACCCTGGTCACCTTCAAGGAT |
| | | GTGTTTGTGGACTTCACCAGAGAAGAGTGGAAGCTGCTGGACACTGCCC |
| | | AGCAAATTGTGTACAGGAATGTCATGCTGGAAAACTACAAGAACCTTGT |
| | | GTCCCTGGGCTACCAGCTCACCAAGCCTGATGTGATCCTGAGACTGGAA |
| | | AAGGGAGAGGAACCCTGGCTTGTGGAGAGGGAAATCCACCAGGAAACCC |
| | | ACCCTGACTCTGAGACTGCCTTTGAGATCAAGTCCAGTGTCTAA |
| 21 | ZFP46025_co6b | ATGGCCCCCAAGAAGAAAAGGAAAGTTGGCATTCATGGAGTGCCTGCAG |
| | (0 CpGs) | CAATGGCTGAAAGGCCCTTCCAATGCAGAATCTGTATGAGAAACTTCTC |
| | | CTGCCCCTCCCATCTGACTAGGCACATCAGGACCCACACTGGAGAAAAG |
| | | CCATTTGCCTGTGACATTTGTGGTAGAAAGTTTGCCCAGAGTGGAGATC |
| | | TCACCAGACACACTAAGATCCACACCCCCAACCCCCATAGAAGAACTGA |
| | | CCCTTCCCACAAGCCATTCCAGTGCAGGATTTGCATGAGAAATTTCAGC |
| | | AAGCATGGGAACCTGTCAGAGCACATCAGAACCCATACTGGGGAAAAGC |
| | | CCTTTGCTTGTGACATCTGTGGAAGAAAATTTGCCAAGAGGTGCAACCT |
| | | GAGATGCCACACCAAAATTCACACAGGCTCCCAGTCCCCATTCCAATGT |
| | | AGAATCTGCATGAGGAAGTTTGCTAGGCAGTTCAACAGGCACCAGCACA |
| | | CCAAGATCCACCTGAGGCAGAAAGATGCAGCCAGAGGCTCTGGAATGGA |
| | | TGCCAAGTCCCTCACTGCCTGGAGCAGAACCCTGGTCACCTTCAAGGAT |
| | | GTGTTTGTGGACTTCACCAGAGAAGAGTGGAAGCTCCTGGACACTGCCC |
| | | AGCAAATTGTGTACAGGAATGTCATGCTTGAGAACTACAAGAACCTGGT |
| | | GTCCCTGGGCTACCAGTTGACCAAGCCTGATGTGATCCTGAGACTGGAA |
| | | AAGGGAGAGGAACCTTGGCTTGTGGAGAGAGAGATCCACCAGGAAACCC |
| | | ACCCTGACTCAGAAACTGCCTTTGAGATCAAGAGCTCAGTGTAA |
| 22 | ZFP46025_co6c | ATGGCCCCCAAGAAGAAGAGAAAAGTGGGCATTCATGGAGTGCCTGCTG |
| | (0 CpGs) | CAATGGCTGAAAGGCCATTCCAGTGTAGAATCTGTATGAGGAACTTCTC |
| | | CTGCCCTTCCCACCTGACCAGACACATTAGGACCCATACTGGAGAGAAG |
| | | CCCTTTGCCTGTGACATCTGTGGCAGGAAGTTTGCCCAGTCAGGGGACC |
| | | TCACTAGGCACACCAAGATCCACACCCCCAACCCACATAGAAGAACTGA |
| | | CCCCAGCCACAAGCCATTCCAATGCAGAATTTGTATGAGAAACTTCTCC |
| | | AAGCATGGCAACCTGTCAGAACACATCAGGACCCACACTGGGGAGAAGC |
| | | CTTTTGCTTGTGACATTTGTGGTAGAAAATTTGCCAAGAGGTGCAACCT |
| | | GAGATGCCACACCAAAATCCACACTGGAAGCCAGAGCCCCTTCCAGTGC |
| | | AGAATCTGCATGAGAAAGTTTGCAAGACAGTTCAACAGACATCAACACA |
| | | CAAAGATCCACCTGAGGCAGAAAGATGCAGCCAGGGGTTCTGGAATGGA |
| | | TGCCAAGTCCCTCACTGCCTGGAGCAGGACCCTGGTCACCTTCAAGGAT |
| | | GTCTTTGTGGACTTCACCAGAGAAGAGTGGAAGCTCCTGGACACTGCCC |
| | | AGCAAATTGTGTACAGAAATGTCATGCTGGAGAACTACAAGAACCTGGT |
| | | GTCCTTGGGCTACCAGCTCACCAAGCCTGATGTGATCCTGAGACTGGAA |
| | | AAGGGAGAGGAACCCTGGCTTGTGGAGAGGGAAATCCACCAGGAAACCC |
| | | ACCCTGACTCAGAGACTGCCTTTGAAATCAAGTCCTCAGTGTAA |
| 23 | ZFP45723_P | ATGGCCCCCAAGAAAAAGCGGAAAGTGGGCATCCACGGGGTACCCGCCG |
| | (34 CpGs) | CTATGGCTGAGAGGCCCTTCCAGTGTCGAATCTGCATGCGTAACTTCAG |
| | | TTCCCCGGAGCAGCTGTCCCGCCACATCCGCACCCACACCGGCGAGAAG |
| | | CCTTTTGCCTGTGACATTTGTGGGAGGAAATTTGCCCAGTGGTCCACCC |
| | | GCAAGCGCCATACCAAGATACACACGCCGAACCCGCACCGCCGCACCGA |
| | | CCCGTCCCACAAGCCCTTCCAGTGTCGAATCTGCATGCGTAACTTCAGT |
| | | AAGCAGGGCAACCTGGTGGAGCACATCCGCACCCACACCGGCGAGAAGC |
| | | CTTTTGCCTGTGACATTTGTGGGAGGAAATTTGCCAAGCGCTGTAACCT |
| | | GCGCTGTCATACCAAGATACACCTGCGCCAAAAAGATGCGGCCCGGGGA |
| | | TCCGGCATGGATGCTAAGTCACTAACTGCCTGGTCCCGGACACTGGTGA |
| | | CCTTCAAGGATGTATTTGTGGACTTCACCAGGGAGGAGTGGAAGCTGCT |
| | | GGACACTGCTCAGCAGATCGTGTACAGAAATGTGATGCTGGAGAACTAT |
| | | AAGAACCTGGTTTCCTTGGGTTATCAGCTTACTAAGCCAGATGTGATCC |
| | | TCCGGTTGGAGAAGGGAGAAGAGCCCTGGCTGGTGGAGAGAGAAATTCA |
| | | CCAAGAGACCCATCCTGATTCAGAGACTGCATTTGAAATCAAATCATCA |
| | | GTTTAA |
| 24 | ZFP45723_co1 | ATGGCACCTAAGAAAAAGAGAAAAGTGGGAATTCATGGAGTACCAGCAG |
| | (0 CpGs) | CTATGGCAGAAAGACCATTCCAGTGCAGAATCTGCATGAGAAACTTCTC |
| | | CAGCCCTGAACAGCTGTCCAGACATATCAGGACCCATACAGGGGAGAAA |
| | | CCCTTTGCCTGTGACATCTGTGGTAGAAAGTTTGCCCAATGGTCCACCA |
| | | GAAAGAGGCACACCAAGATCCACACCCCCAACCCACATAGGAGAACTGA |
| | | CCCTAGCCACAAGCCCTTCCAGTGTAGAATTTGCATGAGGAACTTCAGC |
| | | AAACAGGGCAACCTGGTGGAGCACATCAGAACCCACACTGGAGAAAAGC |
| | | CCTTTGCTTGTGACATTTGTGGAAGGAAGTTTGCCAAGAGGTGCAACCT |
| | | GAGATGCCACACTAAGATCCACCTGAGACAAAAGGATGCTGCCAGGGGC |
| | | TCTGGGATGGATGCCAAGTCCCTCACTGCCTGGAGCAGGACCCTGGTCA |
| | | CCTTCAAGGATGTGTTTGTGGACTTCACCAGAGAAGAGTGGAAGCTCCT |
| | | GGACACTGCCCAGCAAATTGTGTACAGGAATGTCATGTTGGAGAACTAC |
| | | AAGAACCTGGTGTCCCTGGGCTACCAGCTCACCAAGCCTGATGTGATCC |
| | | TGAGGCTGGAAAAGGGAGAAGAACCCTGGCTTGTGGAGAGAGAGATCCA |
| | | CCAGGAAACCCACCCTGACTCAGAGACTGCCTTTGAGATCAAGTCCTCA |
| | | GTCTAA |
| 25 | ZFP45723_co2 | ATGGCCCCTAAGAAGAAGAGAAAAGTTGGTATTCATGGAGTCCCTGCAG |
| | (0 CpGs) | CAATGGCAGAAAGACCATTCCAATGCAGAATCTGCATGAGAAACTTCTC |
| | | CTCCCCTGAACAACTGTCCAGACACATCAGGACCCATACTGGGGAGAAG |
| | | CCCTTTGCCTGTGACATCTGTGGAAGGAAGTTTGCCCAGTGGAGCACCA |
| | | GGAAAAGGCACACCAAGATCCACACCCCAAACCCCCATAGAAGAACTGA |
| | | CCCCAGCCACAAGCCATTCCAGTGCAGAATTTGCATGAGGAACTTCAGC |
| | | AAACAGGGGAACCTGGTGGAGCACATCAGAACCCACACTGGAGAAAAGC |
| | | CTTTTGCTTGTGACATTTGTGGTAGAAAGTTTGCCAAGAGGTGCAACCT |
| | | GAGGTGCCATACCAAGATTCACTTGAGACAGAAAGATGCTGCCAGGGGC |
| | | TCAGGAATGGATGCCAAGTCCCTCACTGCCTGGAGCAGAACCCTGGTCA |
| | | CCTTCAAGGATGTGTTTGTGGACTTCACCAGAGAAGAGTGGAAGCTCCT |
| | | GGACACTGCCCAGCAAATTGTGTACAGGAATGTCATGCTGGAAAACTAC |
| | | AAGAACCTGGTGTCCCTGGGCTACCAGCTCACCAAGCCTGATGTGATCC |
| | | TGAGGCTGGAAAAGGGAGAGGAACCCTGGCTTGTGGAGAGAGAGATCCA |
| | | CCAGGAAACCCACCCTGACTCAGAGACAGCCTTTGAGATCAAGTCCTCT |
| | | GTCTAA |
| 26 | ZFP45723_co3 | ATGGCACCAAAGAAAAAGAGAAAAGTGGGAATTCATGGAGTACCAGCAG |
| | (0 CpGs) | CAATGGCTGAAAGACCCTTCCAATGTAGAATTTGCATGAGGAACTTCAG |
| | | CAGCCCTGAACAGCTCTCAAGGCATATCAGAACCCACACTGGGGAGAAG |
| | | CCTTTTGCCTGTGACATCTGTGGCAGGAAGTTTGCCCAGTGGAGCACCA |
| | | GGAAAAGGCACACCAAGATCCATACCCCCAACCCCCATAGAAGAACAGA |
| | | CCCTTCCCACAAGCCATTCCAGTGCAGAATCTGCATGAGAAACTTCAGC |
| | | AAACAGGGCAACCTGGTGGAACACATCAGGACTCACACTGGAGAAAAGC |
| | | CCTTTGCTTGTGACATTTGTGGGAGAAAGTTTGCCAAGAGGTGCAACCT |
| | | GAGATGCCACACTAAGATCCACCTGAGGCAGAAGGATGCTGCCAGAGGT |
| | | TCTGGAATGGATGCCAAGTCCCTCACTGCCTGGTCCAGAACCCTTGTGA |
| | | CCTTCAAGGATGTGTTTGTGGACTTCACCAGAGAAGAGTGGAAGCTCCT |
| | | GGACACTGCCCAGCAAATTGTGTACAGGAATGTCATGCTGGAGAACTAC |
| | | AAGAACCTGGTGTCCTTGGGCTACCAGCTGACCAAGCCTGATGTCATCC |
| | | TGAGGCTGGAAAAGGGAGAGGAACCCTGGCTTGTGGAGAGAGAGATCCA |
| | | CCAGGAAACCCACCCTGACTCAGAGACTGCCTTTGAGATCAAGTCCTCA |
| | | GTCTAA |
| 27 | ZFP45723_co4 | ATGGCCCCTAAGAAGAAGAGAAAAGTTGGTATACATGGAGTCCCTGCTG |
| | (0 CpGs) | CAATGGCTGAAAGACCATTCCAGTGCAGAATTTGCATGAGAAACTTCTC |
| | | CTCCCCTGAACAACTGTCCAGGCACATTAGGACCCACACTGGAGAGAAG |
| | | CCCTTTGCTTGTGACATCTGTGGTAGAAAGTTTGCCCAGTGGAGCACCA |
| | | GGAAAAGGCACACCAAAATCCACACCCCAAACCCCCATAGAAGAACTGA |
| | | CCCCTCCCATAAGCCCTTCCAGTGTAGAATCTGCATGAGGAACTTCAGC |
| | | AAACAGGGGAACCTGGTGGAACACATCAGGACCCATACTGGGGAGAAGC |
| | | CTTTTGCCTGTGACATTTGTGGAAGGAAGTTTGCCAAGAGATGCAACCT |
| | | GAGATGCCACACCAAGATCCACCTGAGACAAAAGGATGCAGCCAGAGGC |
| | | TCAGGAATGGATGCCAAGTCCCTCACTGCCTGGAGCAGAACCCTGGTCA |
| | | CCTTCAAGGATGTGTTTGTGGACTTCACTAGAGAAGAGTGGAAGCTCCT |
| | | GGACACTGCCCAGCAAATTGTGTACAGGAATGTCATGTTGGAGAACTAC |
| | | AAGAACCTGGTGTCCCTGGGCTACCAGCTCACCAAGCCTGATGTGATCC |
| | | TGAGGCTGGAAAAGGGAGAGGAACCCTGGCTTGTGGAGAGGGAAATCCA |
| | | CCAGGAAACCCACCCAGACTCAGAGACAGCCTTTGAGATCAAGTCCTCT |
| | | GTCTAA |
| 28 | ZFP45723_co5 | ATGGCCCCAAAGAAAAAGAGAAAAGTAGGCATTCATGGAGTCCCAGCAG |
| | (0 CpGs) | CAATGGCAGAAAGACCATTCCAGTGCAGAATCTGCATGAGAAACTTCTC |
| | | CTCCCCTGAACAATTGTCCAGGCACATCAGGACCCACACAGGGGAGAAG |
| | | CCCTTTGCTTGTGACATCTGTGGTAGAAAGTTTGCCCAGTGGTCCACCA |
| | | GAAAGAGACACACCAAGATCCATACCCCCAACCCCCATAGAAGAACTGA |
| | | CCCTAGCCACAAGCCCTTCCAGTGTAGAATTTGCATGAGGAACTTCTCC |
| | | AAACAAGGGAACCTGGTGGAACATATCAGGACTCACACTGGAGAGAAGC |
| | | CTTTTGCCTGTGACATTTGTGGGAGGAAGTTTGCCAAGAGGTGCAACCT |
| | | GAGATGCCACACTAAGATCCACCTGAGGCAGAAAGATGCTGCCAGGGGC |
| | | TCAGGAATGGATGCCAAGTCCCTCACTGCCTGGAGCAGAACCCTGGTCA |
| | | CCTTCAAGGATGTGTTTGTGGACTTCACCAGAGAAGAGTGGAAGCTCCT |
| | | GGACACTGCCCAGCAAATTGTGTACAGGAATGTCATGCTGGAGAACTAC |
| | | AAGAACCTGGTGTCCCTGGGCTACCAGCTCACCAAGCCTGATGTGATCC |
| | | TGAGGCTGGAAAAGGGAGAGGAACCCTGGCTTGTGGAGAGAGAAATCCA |
| | | CCAGGAAACCCACCCTGACTCTGAGACTGCCTTTGAGATCAAGAGCTCA |
| | | GTCTAA |
| 29 | ZFP45723_co6 | ATGGCACCTAAGAAAAAGAGAAAAGTGGGCATACATGGAGTCCCAGCAG |
| | (0 CpGs) | CAATGGCAGAAAGACCATTCCAGTGCAGAATCTGCATGAGAAACTTCTC |
| | | CTCCCCTGAACAACTGTCCAGACACATCAGGACTCATACTGGGGAAAAG |
| | | CCCTTTGCCTGTGACATCTGTGGTAGAAAGTTTGCCCAGTGGTCCACTA |
| | | GGAAAAGGCACACCAAGATCCACACCCCCAACCCACATAGAAGAACTGA |
| | | CCCCTCCCACAAGCCCTTCCAATGTAGAATTTGCATGAGGAACTTCAGC |
| | | AAACAGGGCAACCTGGTGGAGCATATCAGGACCCACACTGGGGAGAAGC |
| | | CTTTTGCTTGTGACATTTGTGGAAGGAAGTTTGCCAAGAGATGCAACCT |
| | | CAGATGCCACACTAAGATCCACCTGAGGCAGAAGGATGCTGCCAGGGGT |
| | | TCTGGAATGGATGCCAAGTCCCTCACTGCCTGGAGCAGAACCCTTGTGA |
| | | CCTTCAAGGATGTGTTTGTGGACTTCACCAGAGAAGAGTGGAAGCTGCT |
| | | GGACACAGCCCAACAGATTGTGTACAGGAATGTCATGCTGGAGAACTAC |
| | | AAGAACTTGGTCAGCCTGGGCTACCAGCTCACCAAGCCTGATGTGATCC |
| | | TGAGACTGGAAAAGGGAGAGGAACCCTGGCTTGTGGAGAGGGAAATCCA |
| | | CCAGGAAACCCACCCTGACTCAGAGACTGCCTTTGAGATTAAGAGCTCA |
| | | GTCTAA |

The polynucleotides encoding the repressors described herein may be delivered using any suitable expression vector, including but not limited to viral (e.g., AAV, Ad, HSV1 etc.) and non-viral vectors (e.g., mRNA, plasmid, minicircle, etc.). Non-viral delivery mechanisms include, for example, lipid nanoparticles, EVs, liposomes, among others. The expression vectors may include additional elements such as a nuclear localization signal (NLS) and/or promoter to drive expression of the repressor (e.g., a constitutive promoter such as the PGK, UBC, EFS or EF1alpha promoter). One or more polynucleotides (e.g., expression vectors) of the same or different form (e.g., viral and/or non-viral vectors) may be delivered to the subject and may be formulated in one or more pharmaceutical compositions. The polynucleotides described herein may be maintained episomally (extra-chromosomally) and/or may be stably integrated into a cell following delivery.

In certain embodiments, the fusion protein comprises a DNA-binding domain and a nuclease domain to create functional entities that are able to recognize their intended nucleic acid target through their engineered (ZFP) DNA binding domains and create nucleases (e.g., zinc finger nuclease) cause the DNA to be cut near the DNA binding site via the nuclease activity.

Expression of the fusion proteins may be under the control of a constitutive promoter or an inducible promoter. In certain embodiments, the promoter self-regulates expression of the fusion protein, for example via inclusion of high affinity binding sites. See, e.g., U.S. Pat. No. 9,624,498.

### Delivery

The proteins and/or polynucleotides (e.g., HTT repressors) and compositions comprising the proteins and/or polynucleotides described herein may be delivered to a target cell by any suitable means including, for example, by injection of proteins, via mRNA and/or using an expression construct (e.g., plasmid, lentiviral vector, AAV vector, Ad vector, exosome, extracellular vesicles (Herrmann, 2021, Nature Nanotechnology, 16, pages 748-759 (2021). In some embodiments, the repressor is delivered using AAV1, AAV2, AAV5, AAV7, AAV9 or AAVrh10. In some embodiments, the repressor is delivered using AAV1. In some embodiments, the repressor is delivered using AAV2. In some embodiments, the repressor is delivered using AAV5. In some embodiments, the repressor is delivered using AAV7. In some embodiments, the repressor is delivered using AAV9. In some embodiments, the repressor is delivered using AAVrh10. In some embodiments, the repressor is delivered using AAV that penetrates the blood brain barrier, i.e., BBB penetrant-AAV. Exemplary BBB penetrant AAV include, but are not limited to, VCAP-101, VCAP-102, 9P801, VCAP-100, VCAP-103, PAL1A, PAL1B, PAL1C, PAL2, CereAAV, Dyno bCAP1, AAV.CAP-B10, AAV.CAP-B20, AAV2-BRIN, AAV2-BR1, STAC-BBBR, or AAV-TT, or AAV-BI-hTFR1, among others (e.g. as described in PCT publications WO2022221400A2, WO2023091948A1 and WO2020014471A1, incorporated herein by reference in entirety; Stanton et al., Cell Press Med 4, 31-50; Goertsen et al., Nat. Neuroscience, 2022, 25 (1): 106-115).

Methods of delivering proteins comprising zinc finger proteins as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Any vector systems may be used including, but not limited to, plasmid vectors, retroviral vectors, poxvirus vectors; herpesvirus vectors and adeno-associated virus vectors, virus like particles (VLPs), etc. In some embodiments, adeno-associated virus vectors are used. In some embodiments, virus like particles (VLPs) are used. In some embodiments, lentiviral vectors or adenovirus vectors are used. See, also, U.S. Pat. Nos. 8,586,526; 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more DNA-binding protein-encoding sequences. Thus, when one or more HTT repressors are introduced into the cell, the sequences encoding the protein components and/or polynucleotide components may be carried on the same vector or on different vectors. When multiple vectors are used, each vector may comprise a sequence encoding one or multiple HTT repressors or components thereof.

Conventional viral and non-viral based gene transfer methods can be used to introduce nucleic acids encoding engineered HTT repressors in cells (e.g., mammalian cells) and target tissues. Such methods can also be used to administer nucleic acids encoding such repressors (or components thereof) to cells in vitro. In certain embodiments, nucleic acids encoding the repressors are administered for in vivo or ex vivo gene therapy uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson (1992) Science 256:808-813; Nabel & Felgner (1993) TIBTECH 11:211-217; Mitani & Caskey (1993) TIBTECH 11:162-166; Dillon (1993) TIBTECH 11:167-175; Miller (1992) Nature 357:455-460; Van Brunt (1988) Biotechnology 6 (10): 1149-1154; Vigne (1995) Restorative Neurology and Neuroscience 8:35-36; Kremer & Perricaudet (1995) British Medical Bulletin 51 (1): 31-44; Haddada et al. in Current Topics in Microbiology and Immunology Doerfler and Böhm (eds.) (1995); and Yu et al. (1994) Gene Therapy 1:13-26.

Methods of non-viral delivery of nucleic acids include electroporation, lipofection, microinjection, biolistics, virosomes, liposomes, immunoliposomes, polycation or lipid: nucleic acid conjugates, naked DNA, naked RNA, artificial virions, and agent-enhanced uptake of DNA. Sonoporation using, e.g., the Sonitron 2000 system (Rich-Mar) can also be used for delivery of nucleic acids. In a preferred embodiment, one or more nucleic acids are delivered as mRNA. Also preferred is the use of capped mRNAs to increase translational efficiency and/or mRNA stability. Especially preferred are ARCA (anti-reverse cap analog) caps or variants thereof. See U.S. Pat. Nos. 7,074,596 and 8,153,773, incorporated by reference herein.

Additional exemplary nucleic acid delivery systems include those provided by Amaxa Biosystems (Cologne, Germany), Maxcyte, Inc. (Rockville, Maryland), BTX Molecular Delivery Systems (Holliston, MA) and Copernicus Therapeutics Inc, (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™ and Lipofectamine™ RNAiMAX). Cationic and neutral lipids that are suitable for efficient receptor-recognition lipofection of polynucleotides include those of Felgner, International Patent Publication Nos. WO 91/17424 and WO 91/16024. Delivery can be to cells (ex vivo administration) or target tissues (in vivo administration).

The preparation of lipid: nucleic acid complexes, including targeted liposomes such as immunolipid complexes, is well known to one of skill in the art (see, e.g., Crystal (1995) Science 270:404-410; Blaese et al. (1995) Cancer Gene Ther. 2:291-297; Behr et al. (1994) Bioconjugate Chem. 5:382-389; Remy et al. (1994) Bioconjugate Chem. 5:647-654; Gao et al. (1995) Gene Therapy 2:710-722; Ahmad et al. (1992) Cancer Res. 52:4817-4820; U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et al. (2009) Nature Biotechnology 27 (7): 643).

The use of RNA or DNA viral based systems for the delivery of nucleic acids encoding engineered ZFPs take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of ZFPs include, but are not limited to, retroviral, lentivirus, adenoviral, adeno-associated, vaccinia and herpes simplex virus vectors, Virus-Like Particles (VLPs) for gene transfer. High transduction efficiencies have been observed in many different cell types and target tissues. Vectors (e.g., retroviruses, adenoviruses, liposomes, VLPs, etc.) containing therapeutic ZFP nucleic acids can be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Adenoviral based vectors do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al. (1987) Virology 160:38-47; U.S. Pat. No. 4,797,368; International Patent Publication No. WO 93/24641; Kotin (1994) Human Gene Therapy 5:793-801; Muzyczka (1994) J. Clin. Invest. 94:1351). Construction of recombinant AAV vectors are described in a number of publications, including U.S. Pat. No. 5,173,414; Tratschin et al. (1985) Mol. Cell. Biol. 5:3251-3260; Tratschin et al. (1984) Mol. Cell. Biol. 4:2072-2081; Hermonat & Muzyczka (1984) PNAS 81:6466-6470; and Samulski et al. (1989) J. Virol. 63:03822-3828.

Recombinant adeno-associated virus vectors (rAAV) are a promising alternative gene delivery systems. Many AAV serotypes, including AAV1, AAV2, AAV3, AAV4, AAV5, AAV6, AAV7, AAV8, AAV 8.2, AAV9, AAV10, AAV11, AAV12, AAV13 and AAVrh10 and pseudotyped AAV such as AAV2/8, AAV2/5 and AAV2/6 can also be used in accordance with the present invention. In addition, shuffled AAVs or synthetic AAVs can also be used, with preferred tropism, such as blood-brain-barrier (BBB) crossing and liver de-targeting. In some embodiments, a BBB-penetrant AAV capsid is used. In some embodiments, AAV9 is used. In some embodiments, AAV5 is used. In some embodiments, the BBB penetrant AAVs used are any one of VCAP-101, VCAP-102, 9P801, VCAP-100, VCAP-103, PAL1A, PAL1B, PAL1C, PAL2, CereAAV, Dyno bCAP1, AAV.CAP-B10, AAV.CAP-B20, AAV2-BRIN, AAV2-BR1, STAC-BBB®, or AAV-TT, or AAV-BI-hTFR1, among others.

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging, and integration into the host genome if in the presence of AAV replication proteins. Viral genes is supplemented in a cell line in trans, which contains a helper plasmid encoding the other AAV genes, namely rep and cap, but lacking ITR sequences. The cell line is also infected with adenovirus as a helper. The helper virus promotes replication of the AAV genome and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. In some embodiments, the AAV is produced at a large scale by using a baculovirus expression vector system (BEVS). (Sandro et al., Methods Mol Biol., 2019; 1937:91-99).

In many gene therapy applications, it is desirable that the gene therapy vector be delivered with a high degree of tropism to a particular tissue type. Accordingly, a viral vector can be modified to have tropism for a given cell type by expressing a ligand as a fusion protein with a viral coat protein on the outer surface of the virus. The ligand is chosen to have affinity for a receptor known to be present on the cell type of interest. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration. In some embodiments, the administration is intravenous, intraperitoneal, intramuscular, subdermal, or intracranial infusion, intranasal, including direct injection into the brain, or topical application, as described below.

In certain embodiments, the compositions as described herein (e.g., polynucleotides and/or proteins) are delivered directly in vivo. The compositions (cells, polynucleotides and/or proteins) may be administered directly into the central nervous system (CNS), including but not limited to direct injection into the brain or spinal cord. One or more areas of the brain may be targeted, including but not limited to, the striatum and/or the caudate, putamen, thalamus, or globus pallidus. In some embodiments, the compositions are delivered to skeletal muscle. Alternatively or in addition to CNS delivery, the compositions may be administered systemically. In some embodiments, the administration is intravenous. In some embodiments, the administration is intraperitoneal. In some embodiments, the administration is intracardial. In some embodiments, the administration is intramuscular. In some embodiments, the administration is intranasal. In some embodiments, the administration is intrathecal. In some embodiments, the administration is subdermal. In some embodiments, the administration is via intracranial infusion. Alternatively, the AAV may be delivered via focused ultrasound. Methods and compositions for delivery of compositions as described herein directly to a subject (including directly into the CNS) include but are not limited to direct injection (e.g., stereotactic injection) via needle assemblies. In some embodiments, convection-enhanced delivery is used, for example, that generates a pressure gradient at the tip of an infusion catheter to deliver therapeutics directly through the interstitial spaces of the central nervous system. In some embodiments, neuronavigation systems are employed, for example, robotic navigation systems, stereotactic frames, frameless navigation systems, the ClearPoint System or other stereotactic neuronavigation systems are used together with appropriate imaging systems, for example MRI or CT for precise delivery. Delivery methods are described, for example, in U.S. Pat. Nos. 7,837,668; 8,092,429, relating to delivery of compositions (including expression vectors) to the brain and U.S. Patent Publication No. 2006/0239966, incorporated herein by reference in their entireties.

The effective amount to be administered may vary from patient to patient and according to the mode of administration and site of administration. Accordingly, effective amounts can be determined by one of ordinary skill in the art. After allowing sufficient time for expression of the repressor (typically 4-15 days, for example), analysis of the serum or other tissue levels of the therapeutic polypeptide and comparison to the initial level prior to administration will determine whether the amount being administered is too low, within the right range or too high. In certain embodiments, when using a viral vector such as AAV, the dose administered is between 1×10⁸ and 5×10¹⁵ vg/ml (or any value therebetween), even more preferably between 1×10¹³ and 1×10¹⁴vg/ml (or any value therebetween), even more preferably between 1×10¹² and 1×10¹³ vg/ml (or any value therebetween).

To deliver ZFPs using recombinant adeno-associated viral (rAAV) vectors directly to the human brain, a dose range of 1×10⁸-5×10¹⁵ vg/mL (or any value therebetween, including for example anywhere between 1×10¹² and 1×10¹⁴ vg/ml or anywhere 1×10¹² and 1×10¹³ vg/mL) vector genome per striatum can be applied. A dose range of 1×10⁸ and 5×10¹⁵ vg/kg or vg/striatum (or any value therebetween), even more preferably between 1×10⁸ and 1×10¹⁴vg/kg or vg/striatum (or any value therebetween), even more preferably between 1×10⁸ and 1×10¹³ vg/kg or vg/striatum (or any value therebetween). As noted, dosages may be varied for other brain structures and for different delivery protocols. Methods of delivering rAAV vectors directly to the brain are known in the art. See, e.g., U.S. Pat. Nos. 9,089,667; 9,050,299; 8,337,458; 8,309,355; 7,182,944; 6,953,575; and 6,309,634.

In some embodiments, vectors can be delivered to cells ex vivo, such as cells explanted from an individual patient (e.g., hematopoietic stem cells, lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In some embodiments, cells are isolated from the subject organism, transfected with at least one HTT repressor or component thereof and re-infused back into the subject organism (e.g., patient).

In some embodiments, VLPs are used to deliver nucleic acid. Virus-like particles (VLPs) are protein complexes similar to native virus particles but do not contain the viral genome so they cannot replicate. Still, VPLs can mimic viral antigenicity without being pathogenic. VLPs consist of one or more natural or artificial protein units in multiple copies and can occur naturally (e.g., poliovirus empty capsids outside of the cell), or be recombinantly produced by expressing the proteins required for VLP production (e.g., vesiculovirus glycoproteins). Some VLPs self-assemble from a single type of coat protein (e.g., L1 of human papillomavirus); some VLPs require several structural proteins (e.g., bluetongue virus VLPs); some VPLs require a combination of structural and non-structural proteins (e.g., poliovirus VLPs). Their repetitive surface structure and size of 20-200 nm make VLPs highly immunogenic, efficient at presenting foreign antigens, such as RNAs that can be loaded on the surface and capable of inducing a strong humoral and cellular immune response.

In some embodiments, non-viral delivery approaches are used. In some embodiments, non-viral nucleic acid vectors are nanoparticles, which can be organic or inorganic. Nanoparticles are well known in the art. Any suitable nanoparticle design can be used to deliver the gene therapy constructs or encoded ZFP-TF proteins of the present disclosure. For instance, organic (e.g., lipid and/or polymer) nanoparticles can be suitable for use as delivery vehicles in certain embodiments of this disclosure. Exemplary lipids for use in nanoparticle formulations are shown in Table 4 (below).

TABLE 4
lists exemplary lipids for nanoparticle formulations
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 20c | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]53rimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-N′-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | Dlin-KC2- | Cationic |
| | DMA | |
| dilinoleyl-methyl-4-dimethylaminobutyrate | Dlin-MC3- | Cationic |
| | DMA | |

TABLE 5
lists exemplary polymers for use in nanoparticle formulations.
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

TABLE 6
summarizes delivery methods for a polynucleotide encoding a ZFP-TF described herein.

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

In other embodiments, one or more nucleic acids of the HTT repressor are delivered as mRNA. Also preferred is the use of capped mRNAs to increase translational efficiency and/or mRNA stability. Especially preferred are ARCA (anti-reverse cap analog) caps or variants thereof. See U.S. Pat. Nos. 7,074,596 and 8,153,773, incorporated by reference herein in their entireties. Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al. (1992) J. Exp. Med. 176:1693-1702).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells) (see Inaba et al. (1992) J. Exp. Med. 176:1693-1702).

Stem cells that have been modified may also be used in some embodiments. For example, neuronal stem cells that have been made resistant to apoptosis may be used as therapeutic compositions where the stem cells also contain the ZFP TFs of the invention. Resistance to apoptosis may come about, for example, by knocking out BAX and/or BAK using BAX- or BAK-specific ZFNs (see U.S. Pat. No. 8,597,912) in the stem cells, or those that are disrupted in a caspase, again using caspase-6 specific ZFNs for example. These cells can be transfected with the ZFP TFs that are known to regulate mutant or wild-type HTT.

Methods for introduction of DNA into hematopoietic stem cells are disclosed, for example, in U.S. Pat. No. 5,928,638. Vectors useful for introduction of transgenes into hematopoietic stem cells, e.g., CD34 cells, include adenovirus Type 35.

Vectors suitable for introduction of transgenes into immune cells (e.g., T-cells) include non-integrating lentivirus vectors. See, for example, Naldini et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

As noted above, the disclosed methods and compositions can be used in any type of cell including, but not limited to, prokaryotic cells, fungal cells, Archaeal cells, plant cells, insect cells, animal cells, vertebrate cells, mammalian cells and human cells. Suitable cell lines for protein expression are known to those of skill in the art and include, but are not limited to COS, CHO (e.g., CHO-S, CHO-K1, CHO-DG44, CHO-DUXB11), VERO, MDCK, WI38, V79, B14AF28-G3, BHK, HaK, NS0, SP2/0-Ag14, HeLa, HEK293 (e.g., HEK293-F, HEK293-H, HEK293-T), perC6, insect cells such as Spodoptera fugiperda (Sf), and fungal cells such as Saccharomyces, Pischia and Schizosaccharomyces. Progeny, variants and derivatives of these cell lines can also be used. In a preferred embodiment, the methods and composition are delivered directly to a brain cell, for example in the striatum.

### Applications

HTT-binding molecules (e.g., ZFPs) and the codon-optimized gene therapy constructs encoding them, as described herein, can be used for a variety of applications. These applications include therapeutic methods in which an HTT-binding molecule (including a nucleic acid encoding a DNA-binding protein) is administered to a subject (e.g., an AAV such as AAV5, AAV9 or a BBB-penetrant AAV) and used to modulate the expression of a target gene (and hence protein) within the subject. In some embodiments, the modulation is in the form of repression, for example, repression of mHTT that is contributing to an HD disease state. In some embodiments, the modulation is in the form of activation when activation of expression or increased expression of an endogenous cellular gene can ameliorate a diseased state. In still further embodiments, the modulation can be cleavage (e.g., by one or more nucleases), for example, for inactivation of a mutant HTT gene. As noted above, for such applications, the HTT-binding molecules, or more typically, nucleic acids encoding them are formulated with a pharmaceutically acceptable carrier as a pharmaceutical composition.

The HTT-binding molecules, or vectors encoding them, alone or in combination with other suitable components (e.g., liposomes, nanoparticles or other components known in the art), can be made into aerosol formulations (i.e., they can be “nebulized”) to be administered via inhalation. Aerosol formulations can be placed into pressurized acceptable propellants, such as dichlorodifluoromethane, propane, nitrogen, and the like. Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Compositions can be administered, for example, by intravenous infusion, orally, topically, intraperitoneally, intravesically, intracranially, intranasally or intrathecally. In some embodiments, compositions are administered by intravenous infusion. In some embodiments, compositions are administered orally. In some embodiments, compositions are administered topically. In some embodiments, compositions are administered intraperitoneally. In some embodiments, compositions are administered intravesically. In some embodiments, compositions are administered intracranially. In some embodiments, compositions are administered intranasally. In some embodiments, compositions are administered intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a patient should be sufficient to affect a beneficial therapeutic response in the patient over time. The dose is determined by the efficacy and K_{d} of the particular HTT-binding molecule employed, the target cell, and the condition of the patient, as well as the body weight or surface area of the patient to be treated. The size of the dose also is determined by the existence, nature, and extent of any adverse side-effects that accompany the administration of a particular compound or vector in a particular patient.

Beneficial therapeutic response can be measured in a number of ways. For example, improvement in Huntington's associated movement disorders such as involuntary jerking or writhing movements, muscle problems, such as rigidity or muscle contracture (dystonia), slow or abnormal eye movements, impaired gait, posture and balance, difficulty with the physical production of speech or swallowing and the impairment of voluntary movements can be measured. Other impairments, such as cognitive and psychiatric disorders can also be monitored for signs of improvement associated with treatment. The UHDRS scale can be used to quantitate clinical features of the disease. Other biomarkers measurement can also be used for determining outcome, including measurement of mHTT in the CSF.

For patients that are pre-symptomatic, treatment can be especially important because it affords the opportunity to treat the disease prior to the extensive neurodegeneration that occurs in HD. This damage initiates prior to the development of the overt symptoms described above. HD pathology primarily involves the toxic effect of mutant HTT in striatal medium spiny neurons. These medium spiny neurons express high levels of phosphodiesterase 10A (PDE10A) which regulates cAMP and cGMP signaling cascades that are involved in gene transcription factors, neurotransmitter receptors and voltage-gated channels (Niccolini et al. (2015) Brain 138:3016-3029), and it has been shown that the expression of PDE10A is reduced in HD mice and post-mortem studies in humans found the same. Further, positron emission tomography (PET) ligands have been developed that are ligands for the PDE10A enzyme (e.g. ¹¹C-IMA107, see, e.g., Niccolini et al., supra; ¹⁸FMNI-659, see, e.g., Russell et al. (2014) JAMA Neurol 71 (12): 1520-1528), and these molecules have been used to evaluate pre-symptomatic HD patients. The studies have been shown that PDE10A levels are altered in HD patients even before symptoms develop. Thus, evaluation of PDE10A levels by PET can be done before, during and after treatment to measure therapeutic efficacy of the compositions of the invention. “Therapeutic efficacy” can mean improvement of clinical and molecular measurements, and can also mean protecting the patient from any further decreases in medium spiny neuron function or an increase in spiny neuron loss, or from further development of the overt clinical presentations associated with HD.

The following Examples relate to exemplary embodiments of the present disclosure in which the HTT-modulator comprises a zinc finger protein. It will be appreciated that this is for purposes of exemplification only and that other HTT-modulators (e.g., repressors) can be used, including, but not limited to, TALE-TFs, a CRISPR/Cas system, additional ZFPs, ZFNs, TALENs, additional CRISPR/Cas systems (e.g., Cfp systems), homing endonucleases (meganucleases) with engineered DNA-binding domains.

### EXAMPLES

### Example 1: Methods and Materials

### Naming of Constructs

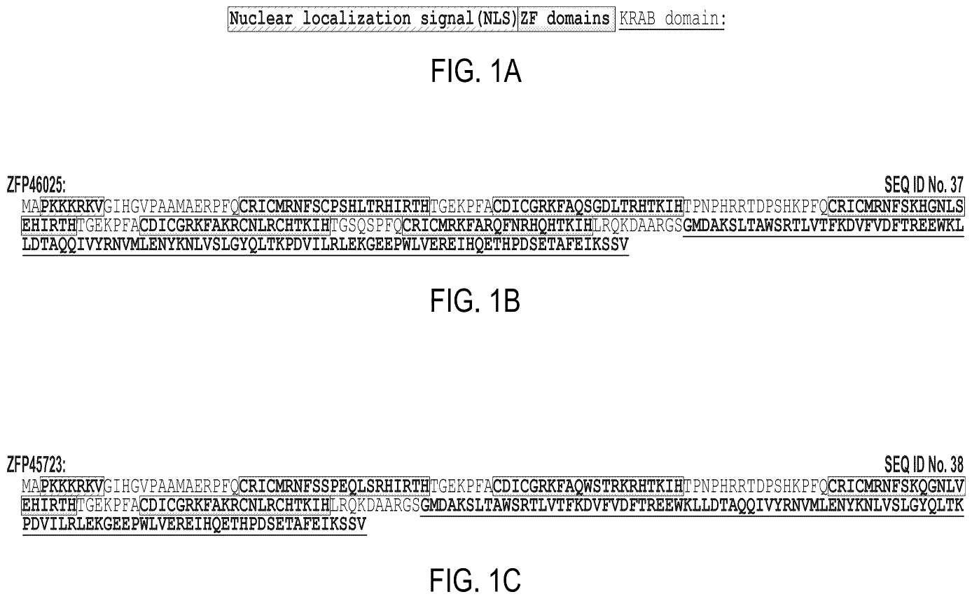

As shown in FIG. 1, the amino acid sequences of ZFP46025 (SEQ ID NO: 37) and ZFP45723 (SEQ ID NO: 38) include an SV40 nuclear localization signal, an array of 4 or 5 zinc finger domains and a KRAB transcriptional repressor domain derived from the human ZNF10/KOX1 protein. Parental nucleotide sequences are referred to as ‘ZFP46025 P’ or ‘ZFP45723_P’, while the Naming of the Codon Optimized Sequences Replaces the ‘P’ with ‘co’ followed by a number (e.g. ZFP46025_col, ZFP45723_co3).

### HEK293 Culture, Transfection and Western Blot Analysis

Approximately 70,000 HEK293 cells (ATCC, catalog #50-188-446FP) were plated per well of a tissue culture treated 24-well plate and transfected 48 hours later with 0.5 ug of plasmid using Lipofectamine™ 3000 Transfection Reagent (ThermoFisher, catalog #L3000001) according to manufacturer's protocol. After approximately 48 hours after transfection, cells were rinsed twice with PBS and lysed with RIPA buffer. Total protein content of the supernatants were measured by Pierce™ Rapid Gold BCA Protein Assay Kit (ThermoFisher, catalog #A53226). Western blots were performed with 10 ug of total protein using primary antibodies against the KRAB domain (ThermoFisher, catalog #PA5-110594) and GAPDH (Cell Signaling Technologies, catalog #97166). Western blot membranes were imaged and analyzed using a LI-COR Odyssey® system.

rAAV Vector Production and Characterization

HEK293 cells were triple transfected with plasmids containing: (1) the AAV2 Rep gene and the AAV9 or AAV.PHP.eB Cap gene, (2) a helper plasmid containing adenoviral genes E2A, E4 and VA, and (3) a transgene plasmid containing AAV2 ITRs flanking a transgene expression cassette. AAV particles were purified either by POROS CaptureSelect AAVX column followed by cesium chloride sedimentation, or by two rounds of cesium chloride sedimentation. Fractions containing mostly full capsids were pooled and subjected to buffer exchange in phosphate buffered saline with 0.001% Pluronic F-68. The genome titer was determined by ddPCR, the purity was assessed by either SDS-PAGE followed by silver staining or CE-SDS to visualize VP1, VP2 and VP3, and the endotoxin was assessed by a lumulus amebocyte lysate assay (Endosafe). Characterized vectors were aliquoted and stored at −80° C.

HD Human iPSC-Derived Cortical Culture, Transduction and PCR Analysis

Human induced pluripotent stem cells (iPSCs) from HD patient with heterozygous polyglutamine (180 repeat) in Htt gene (NINDS Human Cell and Data Repository, #ND36999) were differentiated into cortical neuronal progenitor cells (NPCs) by using a reported method by Shi et al., (Nat Protoc. 2012 October; 7 (10): 1836-4) Poly-D-Lysine (PDL) 96-well plate (Corning, #356640) were coated with 10 μg/mL of laminin (R&D systems, #3400-010-02) for more than 3 h prior to the cell plating. NPCs were seeded onto the laminin-PDL coated 96 well plates at 20,000 cells/well. At 7 days after seeding, AAVs were transduced at MOIs of 1E6, 5E6 and 1E7 and cultured for additional 7 days. Total RNA of each sample was extracted by using RNeasy Micro Kit (Qiagen, #74004) followed by cDNA synthesis with SuperScript VILO cDNA Synthesis Kit (Invitrogen, #11754250). RT-qPCR was performed using SsoAdvanced™ Universal SYBR® Green Supermix (Bio-Rad, #172-5271) and the QuantStudio 12K Flex real-time PCR instrument (Thermofisher). PrimePCR™ SYBR® Green Assay: human GAPDH (Bio-Rad, #qHsaCED0038674) was used for detecting GAPDH. Primer sequences for wild type Htt and mutant Htt and Thermal cycling parameter for each RT-qPCR reaction are shown below.

TABLE 7
Primer sequences

| Target mRNA | forward primer | reverse primer |
|---|---|---|
| Wild type | AGTTTGGAGGGTTTC | TCGACTAAAGCAGGA |
| Htt | TT | TTTCAGG |
| | (SEQ ID NO: 30) | (SEQ ID NO: 31) |
| Mutant Htt | AGTTTGGAGGGTTTC | TCGACTAAAGCAGGA |
| | TC | TTTCAGG |
| | (SEQ ID NO: 32) | (SEQ ID NO: 31) |

TABLE 8
PCR cycles

| Assay | Thermal cycling program |
|---|---|
| mutant Htt | 1) 98° C. for 2 min, 2) 95° C. for 5 sec, |
| | 3) 58.3° C. for 9 sec, 4) Plate Read, 5) |
| | Repeat steps 2 to 4, 45 times, 6) End |
| wild type | 1) 98° C. for 2 min, 2) 95° C. for 5 sec, |
| Htt/GAPDH | 3) 55.6° C. for 9 sec, 4) Plate Read, 5) |
| | Repeat steps 2 to 4, 45 times, 6) End |

ZFP mRNA Quantification by RT-qPCR Analysis

Total RNA isolation and following cDNA synthesis was conducted with above mentioned procedure. RT-qPCR was performed using SsoAdvanced™ Universal SYBR® Green Supermix (Bio-Rad, #172-5271) and the QuantStudio 12K Flex real-time PCR instrument (Thermofisher). Primer sequences for each reaction and Thermal cycling parameter for each RT-qPCR reaction are shown below.

TABLE 9
Primer sequences

| Target mRNA | forward primer | reverse primer |
|---|---|---|
| ZFP 46025 | TTTGTGGACTTCACC | GTTCTCCAGCATCAC |
| parental, | AG | ATT |
| co5 and co6 | (SEQ ID NO: 33) | (SEQ ID NO: 34) |
| ZFP 46025 | TGTATTTGTGGACTT | TGCTGAGCAGTGTCC |
| parental, | CAC | AG |
| co1, co3, | (SEQ ID NO: 35) | (SEQ ID NO: 36) |
| co4, co5b | | |
| and co6b | | |

TABLE 10
PCR conditions

| Assay | Thermal cycling program |
|---|---|
| ZFP 46025 | 1) 98° C. for 2 min, 2) 95° C. for 5 sec, |
| | 3) 58.3° C. for 9 sec, 4) Plate Read, |
| | 5) Repeat steps 2 to 4, 45 times, 6) End |

### ZFP Protein Quantification by Immunocytochemistry (ICC)

HD derived NPCs were cultured for 7 days and transduced each AAV as shown above. After 7 days after transduction, NPCs were fixed with 4% Paraformaldehyde (Fujifilmwako, #163-20145) for 30 minutes at room temperature followed by 1 hr incubation with TritonX-100 (MP Biomedicals, #807423) The plate was incubated with an antibody against the KRAB domain (ThermoFisher, #PA5-110594) for more than 12 hr at 4° C. After wash with D-PBS (Fujifilmwako, #045-29795), the plate was further incubated with goat anti-rabbit IgG (H+L) AF488 (Invitrogen, #A11034), Nuclear staining was conducted using Hoechst33342 (Invitrogen, #H3570) after washing with D-PBS. All data was obtained by CellVoyager 8000 imaging system (Yokogawa). Nuclear area were used as a region of interest (ROI) and were defined by using nuclear staining positive areas. ZFP protein level in nucleus was measured by detecting immunochemistry signal of ZFP overlapped with ROI.

### Example 2: Transfection of HEK293 with ZFP46025 Codon Optimized Variants

To generate codon optimized sequences, the ATUM tool was used (U.S. Pat. Nos. 7,561,972, 7,561,973, 8,126,653, 8,401,798) yielding hundreds of sequences, from which sequences were screened using bioinformatics tools to remove potential splice sites, cryptic promoters, long repeats. From the remaining candidates, sequences were selected containing 6 CpGs or less than 6 CpGs (SEQ ID NOs: 11-16).

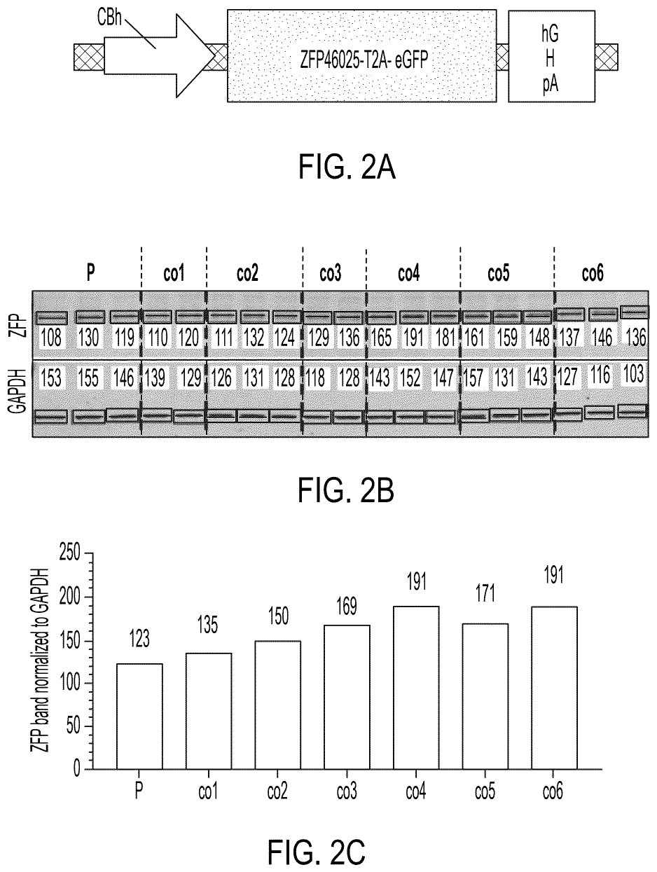

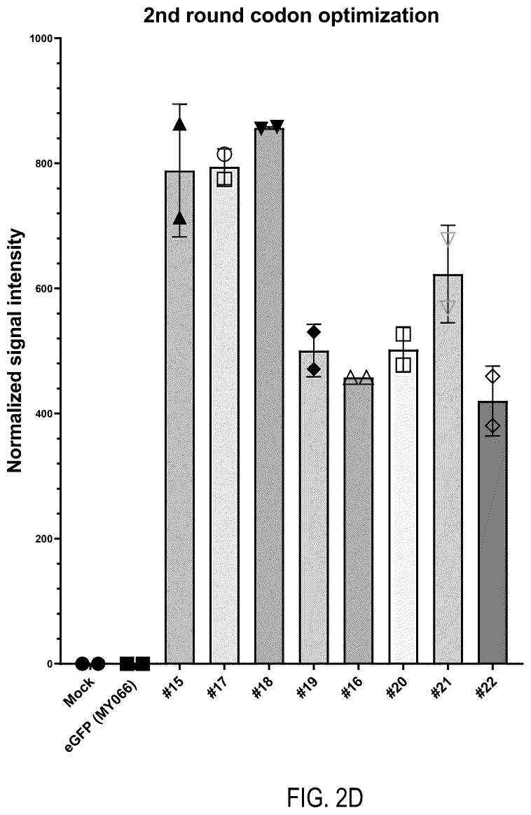

The parental ZFP46025_P (SEQ ID NO 10) has 38 CpGs. Two alternative codon optimized variants (ZFP46025_col and ZFP46025_co2) were designed with 4 CpGs (SEQ ID NO: 11 & 12), ZFP46025_co3 with 6 CpGs (SEQ ID NO: 13), ZFP46025_co4 with 5 CpGs (SEQ ID NO: 14) and ZFP46025_co5 and ZFP46025_co6 with 0 CpGs (SEQ ID NO: 15 & 16). Expression plasmids were constructed with a hybrid chicken beta-actin (CBh) promoter, a human growth hormone (hGH) poly adenylation signal (polyA) and a transgene that includes: either ZFP46025_P or one of the six codon optimized variant sequences fused to a T2A self-cleaving peptide and eGFP (FIG. 2A). Western blot analysis of transfected HEK293 cells demonstrated higher mean ZFP protein expression of the codon optimized variants relative to the parental sequence (FIG. 2B and FIG. 2C).

Two selected sequences, SEQ ID NO: 15 and SEQ ID NO: 16 were again applied to codon optimizing algorithm with the goal to further improve expression. Four candidates SEQ ID NOs: 17-21 were further analyzed.

Expression plasmids were constructed with a hybrid chicken beta-actin (CBh) promoter, a human growth hormone (hGH) poly adenylation signal (polyA) and a transgene that includes: either SEQ ID NO: 15 and SEQ ID NO: 16 or one of the four codon optimized variant sequences fused to a T2A self-cleaving peptide and eGFP (FIG. 2A). GFP signal intensity analysis of transient transfected HEK293 cells demonstrated higher mean ZFP protein expression for some of the codon optimized variants relative to the parental sequence (FIG. 2D)

The results showed that some, but not all codon-optimized sequences showed higher expression than the parental variants. For example, from transfection experiments, it was observed that plasmids with SEQ ID No. 19 and 22 showed decreased expression in a HEK293 assay system.

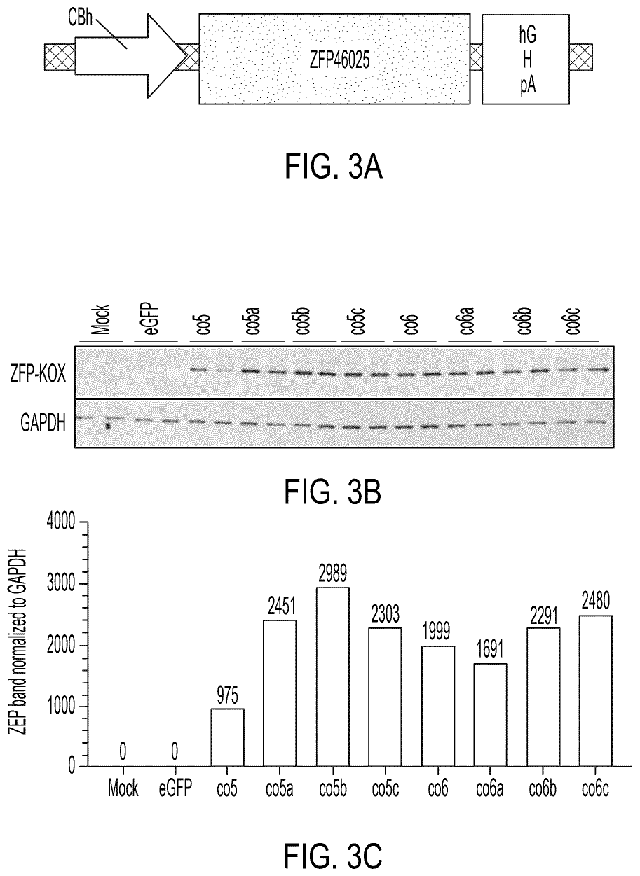

Expression plasmids were constructed with a hybrid chicken beta-actin (CBh) promoter, a human growth hormone (hGH) poly adenylation signal (polyA) and a codon optimized variant sequences as the transgene (FIG. 3A). Western blot analysis of transfected HEK293 cells demonstrated that all three alternative sequences of ZFP46025_co5 had improved ZFP expression, whereas only ZFP46025_co6b and ZFP46025_co6c had higher expression relative to ZFP46025_co6 (FIG. 3B and FIG. 3C).

Overall, transfection in HEK293 cells revealed that a subset of codon optimized sequences showed unexpectedly increased expression relative to wild-type.

### Example 3: Transduction of Human iPSC-Derived Cortical Neurons with ZFP46025 Codon Optimized Variants

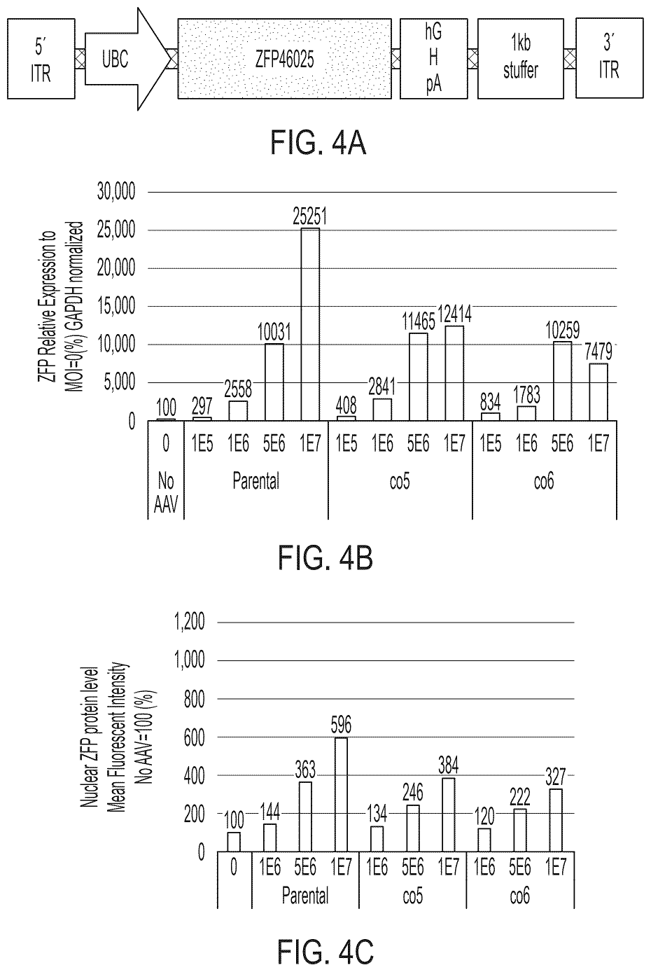

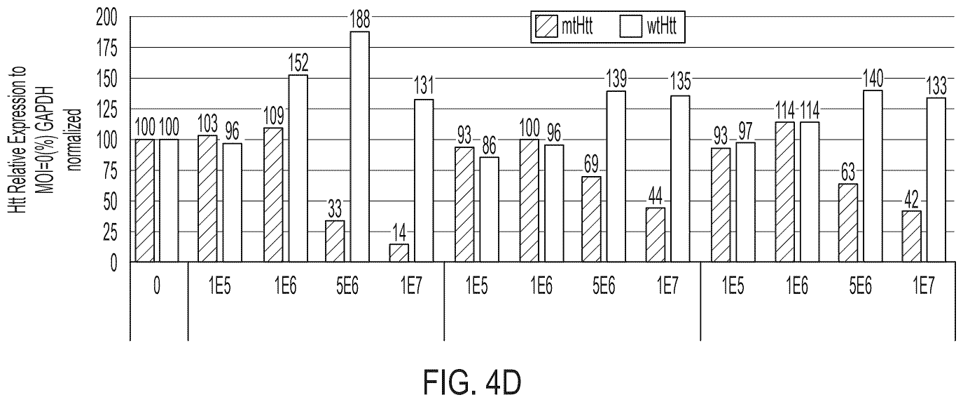

ZFP46025_co5 and ZFP46025_co6 were inserted into an AAV expression plasmid containing AAV2 ITR flanking a human ubiquitin-c (UBC) promoter, a human growth hormone (hGH) poly adenylation signal and a 1 kb stuffer sequence derived hSCNB (intron 5) to prevent mis-packaging (FIG. 4A). Human iPSC-derived cortical neurons were transduced at MOIs of 1E5, 1E6, 5E6 and 1E7. mRNA and protein level of ZFP46025 were determined by RT-qPCR and ICC, respectively (FIG. 4B and FIG. 4C). Compared with AAV with parental ZFP46025, ZFP46025_co5 and ZFP46025_co6 showed lower mRNA and lower protein level. Allele-specific PCR analysis demonstrated reduction of mutant Htt allele mRNA at 5E6 and 1E7 (FIG. 4D).

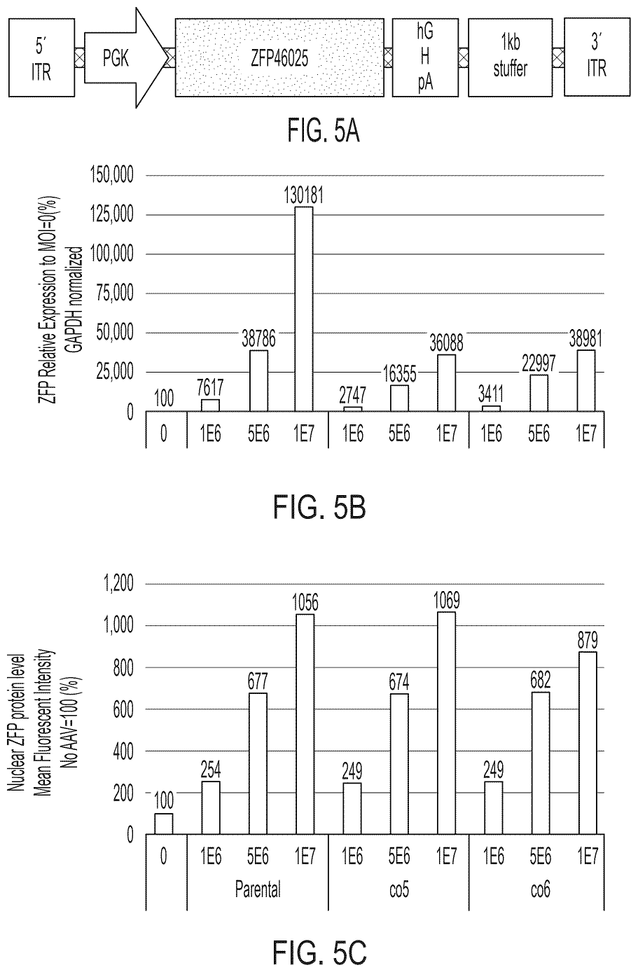

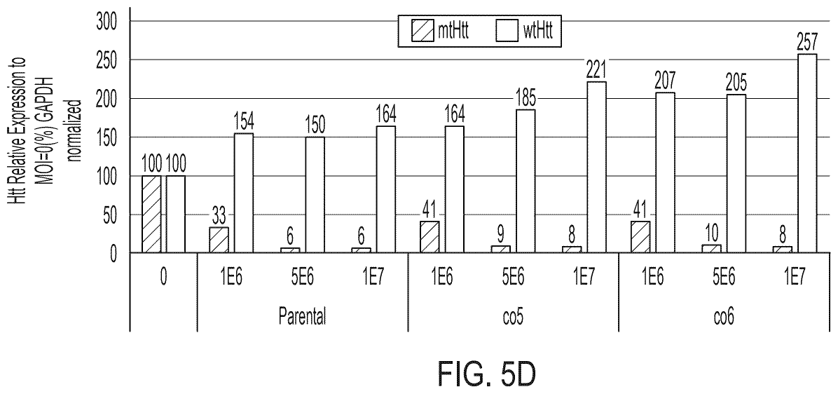

ZFP46025_co5 and ZFP46025_co6 were inserted into an AAV expression plasmid containing AAV2 ITR flanking a human phosphoglycerate kinase 1 (PGK) promoter, a human growth hormone (hGH) poly adenylation signal and a 1 kb stuffer sequence derived hSCNB (intron 5) to prevent mis-packaging (FIG. 5A). Human iPSC-derived cortical neurons were transduced at MOIs of 1E6, 5E6 and 1E7. mRNA and protein level of ZFP46025 were determined by RT-qPCR and ICC, respectively. (FIG. 5B and FIG. 5C) ZFP46025_co5 and ZFP46025_co6 showed comparable mRNA and protein expression with parental ZFP46025. PGK promoter based AAVs achieved higher mRNA and protein level than UBC promoter based ones. Allele-specific PCR analysis demonstrated reduction of mutant Htt allele mRNA at all MOIs (FIG. 5D).

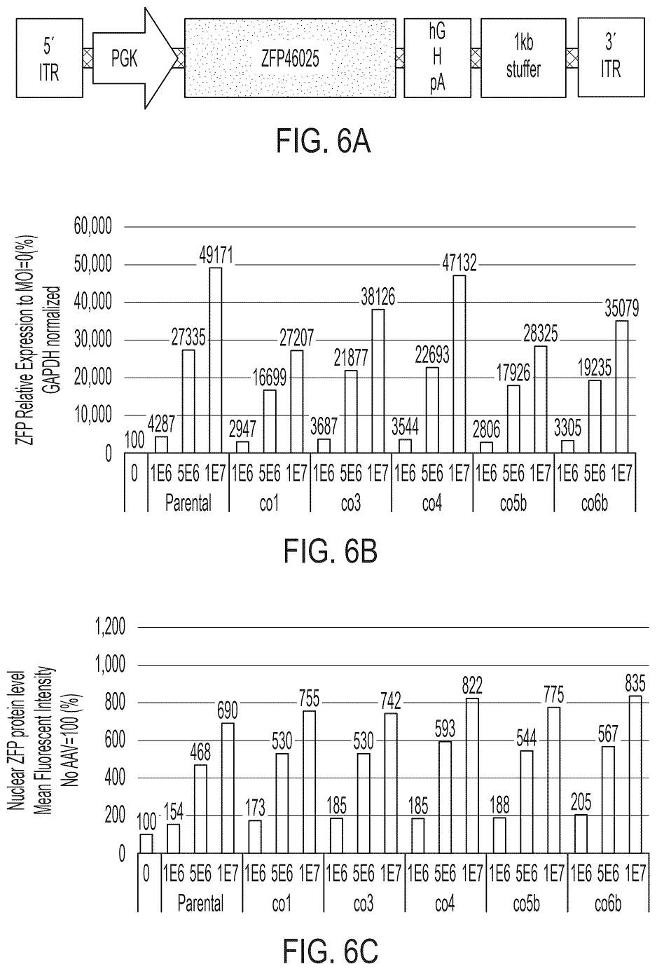

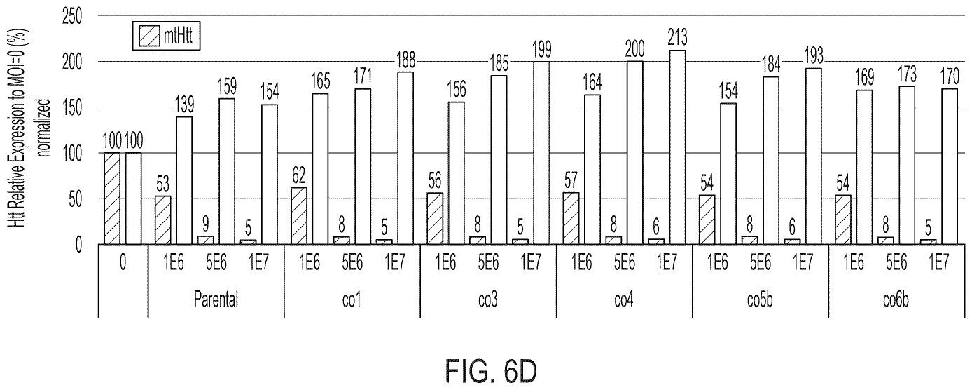

ZFP46025_col (SEQ ID NO: 11), ZFP46025_co3 (SEQ ID NO: 13), ZFP46025_co4 (SEQ ID NO: 14), ZFP46025_co5b (SEQ ID NO: 18) and ZFP46025_co6b (SEQ ID NO: 21) were inserted into an AAV expression plasmid containing AAV2 ITR flanking a human phosphoglycerate kinase 1 (PGK) promoter, a human growth hormone (hGH) poly adenylation signal and a 1 kb stuffer sequence derived hSCNB (intron 5) to prevent mis-packaging (FIG. 6A). Human iPSC-derived cortical neurons were transduced at MOIs of 1E6, 5E6 and 1E7. mRNA and protein level of ZFP46025 were determined by RT-qPCR and ICC, respectively. (FIG. 6B and FIG. 6C) All codon optimized ZFP46025 showed comparable mRNA and protein expression with parental ZFP46025. Allele-specific PCR analysis demonstrated reduction of mutant Htt allele mRNA at all MOIs (FIG. 6D).

### Example 4: Transfection of HEK293 with ZFP45723 Codon Optimized Variants

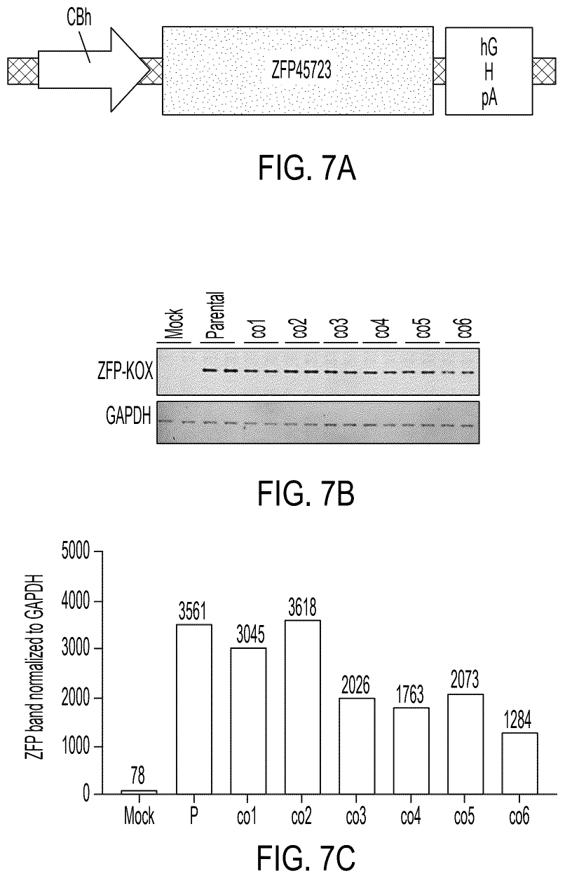

The parental ZFP45723_P (SEQ ID NO 23) has 34 CpGs. Six alternative codon optimized variants were designed with 0 CpGs (SEQ ID NO: 24 to 29). Expression plasmids were constructed with a hybrid chicken beta-actin (CBh) promoter, a human growth hormone (hGH) poly adenylation signal (polyA) and either the parental ZFP45723 or one of the six codon optimized variant sequences (FIG. 7A).

The results showed that Western blot analysis following transfection of HEK293 cells demonstrated similar or reduced protein expression of the codon optimized variants relative to the parental sequence (FIG. 7B and FIG. 7C). This shows that not all codon optimization results in improved expression, as constructs containing co3, co4, co5 or co6 have 50% or even more reduced expression levels, while constructs containing col and co2 showed improved or comparable expression.

### Example 5: Transduction of Human iPSC-Derived Cortical Neurons with ZFP45723 Codon Optimized Variants

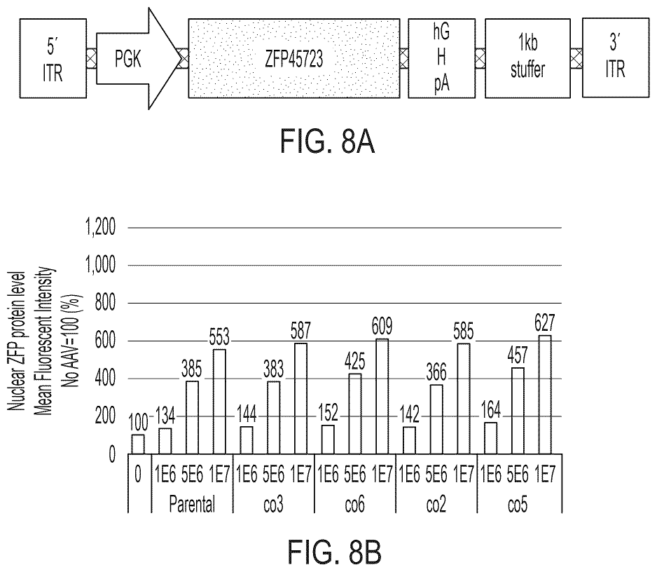

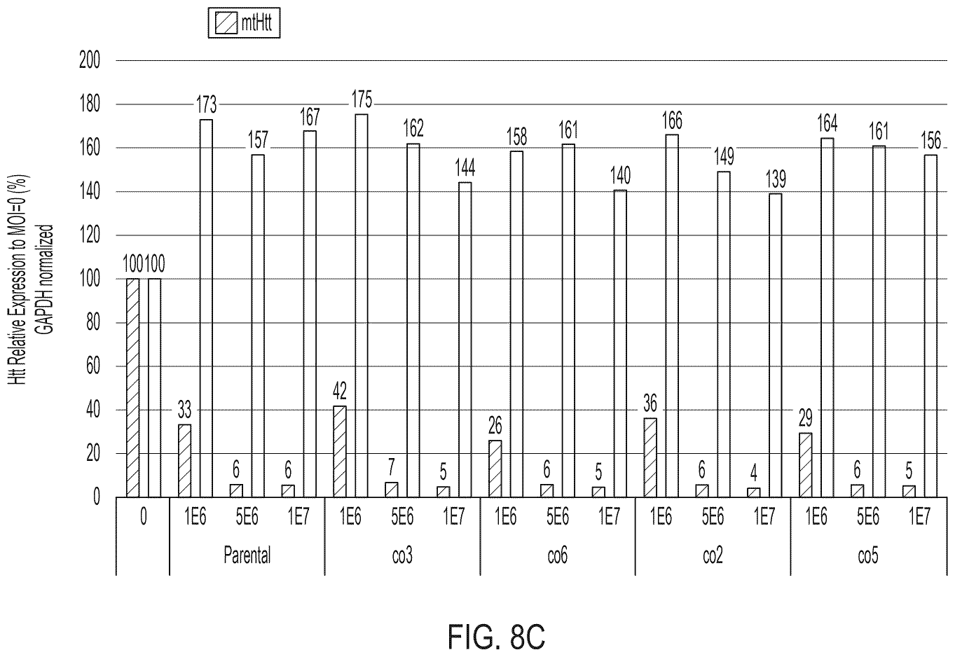

Human iPSC-derived cortical neurons were transduced at MOIs of 1E6, 5E6 and 1E7. Protein level of ZFP45723 was determined by ICC. (FIG. 8B) All codon optimized ZFP45723 (SEQ ID NO: 24-29) showed comparable protein expression with parental ZFP45723 (SEQ ID NO: 23). Allele-specific PCR analysis demonstrated reduction of mutant Htt allele mRNA at all MOIs (FIG. 8C).

### Example 6: ZFP Expression in iPSC-Derived Neurons after AAV Transduction

iPSC-derived neural progenitor stem (NPC) cells were differentiated for 7 days, and then transduced with AAV, with ZFP expression under the control of the UBC promoter. After 7 days, the cells were fixed for data analysis to determine ZFP expression. Fixed cells were stained with an anti-ZNF10 antibody to detect ZFP expression using a fluorescent label. The cells were analyzed in a High Content Imager/CV8000. The graph shows mean fluorescent intensity per well.

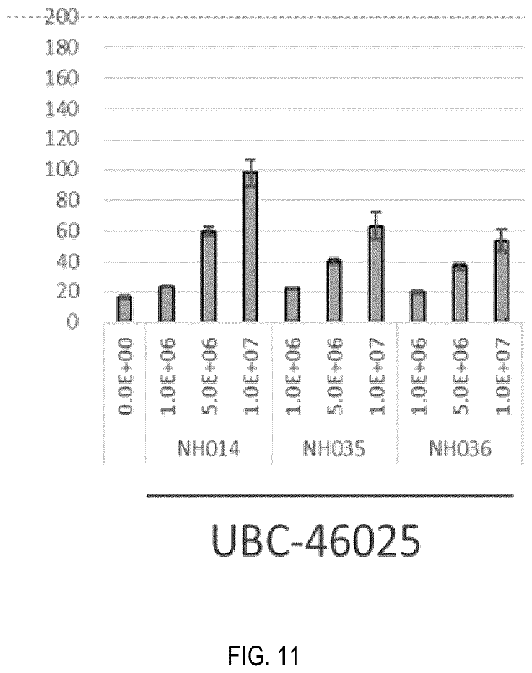

The results in FIG. 11 show that codon optimized sequences SEQ ID NO: 15 (NH035) and SEQ ID NO: 16 (NH035) yield a lower expression compared to the non-codon optimized parental (SEQ ID NO: 10) NH014 AAV.

### Example 7: Evaluation of Several Promoters in the Q175 HD Mouse Model

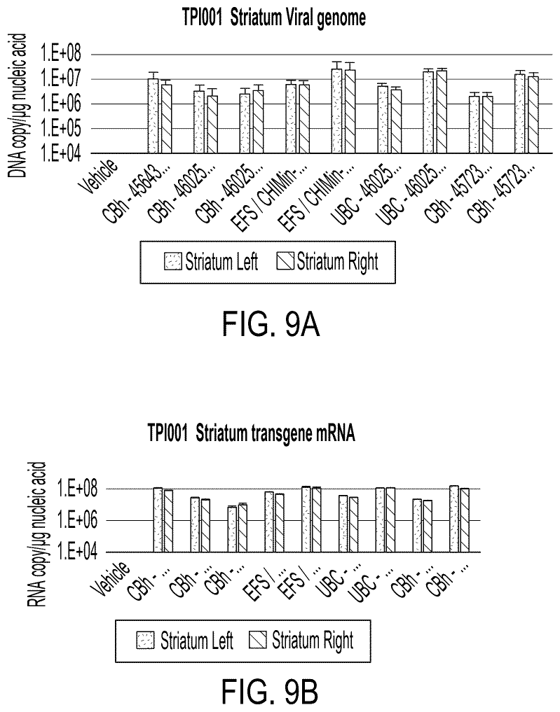

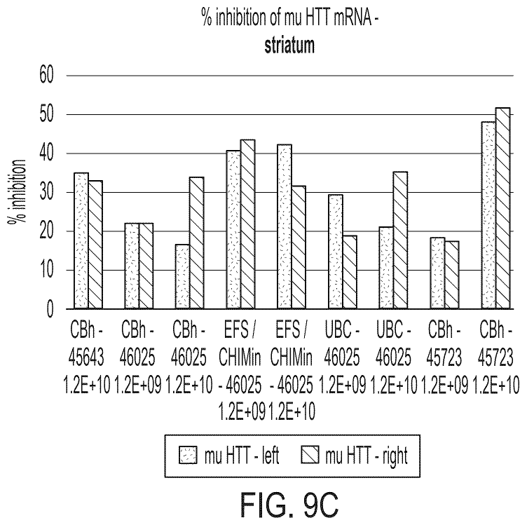

In this example, in vivo activity of two ZFPs (46025 and 47523) with four different promoters CBh, UBC, EFS (EF1alpha promoter short form)/CHIMin, PGK/CHIMin were packaged into AAV9 and in Q175 HD mouse model (Menalled L B et al., 2012, PLOS One 7: e49838). Bilateral stereotactic striatal injections of AAV9 containing the ZFPSs were done at 11 weeks of age. Two AAV doses, 1.2e9 vg and 1.2e10 vg were administered in 2 ul volume per injection site. Stereotaxic coordinates were anterior-posterior [AP], +0.8 mm; medial-lateral [ML], ±1.8 mm; dorsal-ventral [DV], −3.0 mm. Eight weeks after AAV administration mice were sacrificed, and brains were collected for analysis. Vector genome quantification and RT-PCR was carried out using standard methods and under conditions described in the preceding examples. Vector genome (Vg) DNA (FIG. 9A), transgene transcript (FIG. 9B) and effect on mHTT RNA lowering (FIG. 9C) were analyzed.

All promoters tested achieved a mHTT RNA lowering of greater than 30% at the high dose.

### Example 8: Evaluation of PGK and UBC Promoters in the Q175 HD Mouse Model after IV Administration

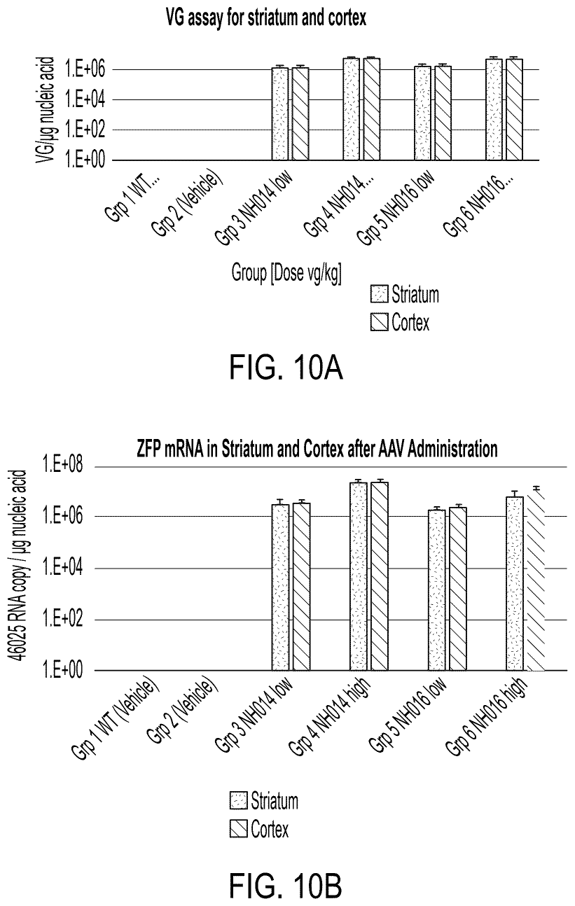

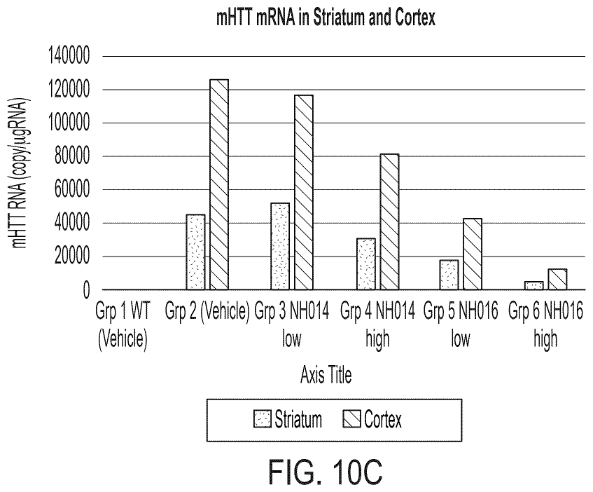

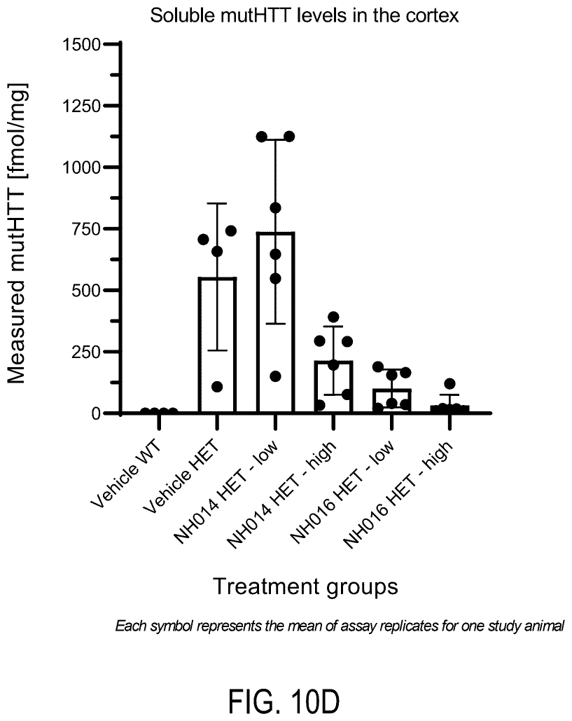

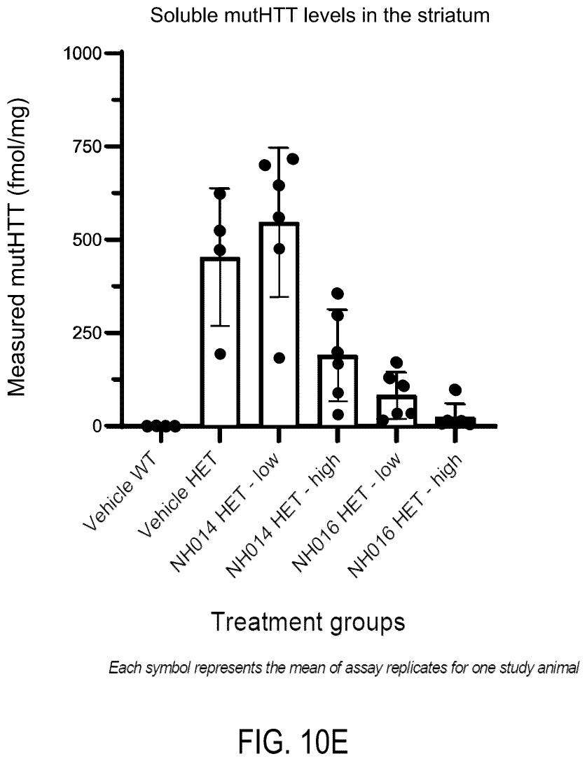

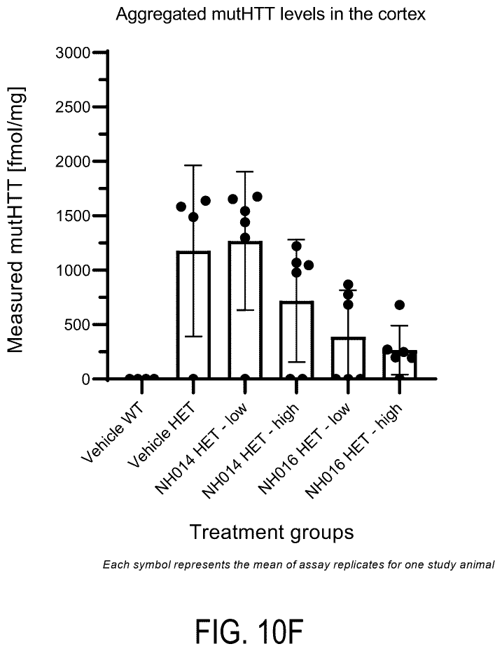

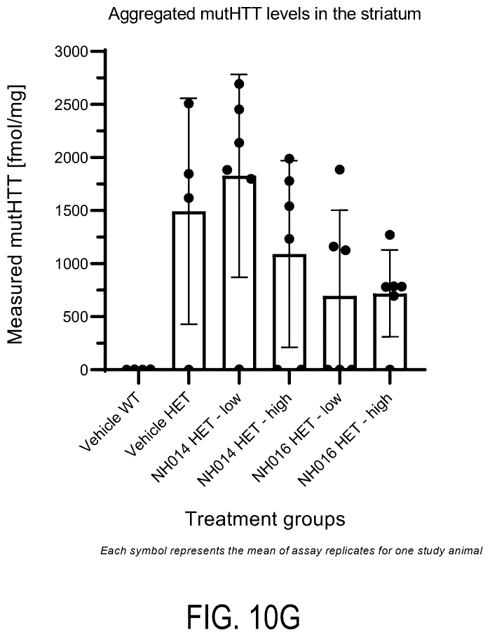

Three-month-old Q175 HD heterozygous mice received intravenous (IV) tail vein administration of buffer, or AAV-PHP.eB-NH014 (UBC-46025) or AAV-PHP.eB-NH016 (PGK-46025) at two doses, 5e12 vg/kg or 4e13 vg/kg. Test articles were administered via a single IV tail vein injection (up to 200 ul). Briefly, the mouse was placed under a heat lamp (approximately 3-5 inches from top of cage) for 30 seconds to dilate lateral tail veins. The mouse was restrained using the mouse tail illuminator apparatus (Braintree Scientific) and the distal injection site on the tail was disinfected with 70% isopropyl alcohol. Test articles were delivered to a lateral tail vein with a 28G 0.5 cc ½ inch U-100 insulin syringe. For treatment groups, 4 males and 4 females were injected, for control groups, 3 males and 3 females. Three months after administration, brains were isolated and analyzed for AAV biodistribution by vector genome (VG) analysis, transgene expression and effect on mutant huntingtin (mHTT) RNA lowering. FIG. 10A shows dose dependent vector genome (DNA) distribution for cortex and striatum, showing a dose-dependent distribution. FIG. 10B shows a dose dependent transgene expression in cortex and striatum, and the results showed that both promoters achieved similar expression levels. The effect on mHTT is shown in FIG. 10C, where a dose-dependent repression of mHTT was detected for both vectors. The PGK promoter performs better than the UBC promoter.

Soluble and aggregated mutant HTT protein was analyzed by immunohistochemistry (IHC) as well as quantified by Meso Scale Discovery (MSD) immunoassays in cortex and striatum of brain. Positive IHC staining was observed in the brain against ZFP in mice administered a high dose (4e13 vg/kg) of AAV-PHP.eB-NH014 (UBC-46025) and at both doses (5e12 vg/kg and 4e13 vg/kg) of AAV-PHP.eB-NH016 (PGK-46025). In the high dose AAV-PHP.eB-NH016 group, ZFP expression was most prominent in the nuclei of neuronal cells.

In mice dosed with AAV-PHP.eB-NH016, the staining was extensive throughout the brain with intensity and distribution increasing with dose. Positive staining against ZFP was negatively correlated with mHTT staining in the both groups (low and high dose) for PHP.eB-NH016. In the high dose group for AAV-PHP.eB-NH016, mHTT was not detectable.

FIG. 10D shows a graph quantifying levels of soluble mHTT in brain cortex as determined using a MSD immunoassay. FIG. 10E shows a graph quantifying levels of soluble mHTT in brain striatum as determined using a MSD immunoassay. FIG. 10F shows a graph quantifying levels of aggregated mHTT in brain cortex as determined using a MSD immunoassay. FIG. 10G shows a graph quantifying levels of aggregated mHTT in striatum as determined using a MSD immunoassay.

The results from FIG. 10D-FIG. 10G showed that AAV-PHP.eB-NH016 (PGK-46025) effectively lowered mHTT soluble and aggregated protein in a dose dependent manner, and that AAV-PHP.eB-NH014 (UBC-46025) lowered soluble aggregated mHTT at the high dose.

Overall, an exemplary ZFP-TF driven by PGK and UBC promoters delivered to mouse by exemplary BBB penetrating AAV vectors, showed dose-dependent repression of mHTT in vivo in a mouse model of Huntington's disease.

TABLE 11
Summary of Immunohistochemistry Scoring

| | | | | | Low dose | | High dose | | Low dose | | High dose | |
|---|---|---|---|---|---|---|---|---|---|---|---|---|
| | | | Q175 | | NH014 | | NH014 | | NH016 | | NH016 | |
| | WT Control | | Control | | (5e12 vg/kg) | | (4e13 vg/kg) | | (5e12 vg/kg) | | (4e13 vg/kg) | |
| | WT | | HET | | HET | | HET | | HET | | HET | |
| | M | F | M | F | M | F | M | F | M | F | M | F |
| Tissue/Diagnosis | 5 | 3 | 8 | 10 | 20 | 13 | 27 | 22 | 31 | 34 | 41 | 40 |
| Brain | N | N | N | N | N | N | N | N | N | N | N | N |
| Brain-ZFP | Neg | Neg | Neg | Neg | Neg | Neg | | | | | | |
| Positive, caudate putamen | | | | | | | | | 1; 2* | | 3; 3 | 3; 2 |
| Positive, thalamus | | | | | | | | | 3; 3 | 3; 3 | 2; 2 | 3; 3 |
| Positive, globus palladus | | | | | | | | | NP | | 2; 3 | 1; 2 |
| Positive, cerebral cortex | | | | | | | | | 2; 3 | 2; 3 | 3; 3 | 3; 3 |
| Positive, septal nuclei | | | | | | | | | 2; 3 | 2; 2 | 2; 3 | 4; 3 |
| Positive, hippocampus | | | | | | | | | 2; 2 | 2; 2 | 3; 3 | 2; 3 |
| Positive, amygdala | | | | | | | | | | 1; 2 | 4; 3 | 2; 2 |
| Positive, midbrain | | | | | | | | | 2; 2 | 2; 2 | 2; 2 | 2; 2 |
| Positive, olfactory bulbs | | | | | | | 2; 3 | 2; 3 | | 1; 3 | 1; 3 | 1; 3 |
| Brain-mHTT | Neg | Neg | | | | | | Neg | | | Neg | Neg |
| Positive, caudate putamen | | | 4; 3 | 4; 2 | 4; 2 | 4; 3 | 4; 3 | | 3; 1 | 3; 1 | | |
| Positive, thalamus | | | 0 | 0 | 2; 1 | 2; 1 | 1; 1 | | 0 | 0 | | |
| Positive, globus palladus | | | NP | NP | NP | NP | 0 | | NP | 0 | | |
| Positive, cerebral cortex | | | 3; 2 | 3; 2 | 3; 2 | 3, 2 | 3; 2 | | 2; 1 | 2; 1 | | |
| Positive, septal nuclei | | | 0 | NP | NP | 3; 2 | 2; 1 | | 0 | 0 | | |
| Positive, hippocampus | | | 3; 2 | 3; 2 | 2; 1 | 4; 2 | 2; 2 | | 2; 1 | 3; 1 | | |
| Positive, amydala | | | 1; 1 | 1; 1 | 1; 1 | 4; 2 | 1; 1 | | 0 | 2; 1 | | |
| Positive, midbrain | | | 0 | 2; 1 | 2; 1 | 2; 1 | 1; 1 | | 0 | 1; 1 | | |
| Positive, olfactory bulbs | | | NP | 2; 1 | 0 | 2; 1 | 0 | | 1; 1 | 1; 1 | | |

Key: N=Normal; Neg-Negative; NP=Not Present in section; * First value represents % positivity of cells, second value is intensity score; % positive cells: 0=0%; 1=<1-5%; 2=6-25%; 3=26-50%; 4=51-75%; 5-76-100%. Intensity score: 1=Weak; 2=Moderate; 3=Strong; 4=Intense.

All patents, patent applications and publications mentioned herein are hereby incorporated by reference for all purposes in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

### NUMBERED EMBODIMENTS

1. A gene therapy construct encoding a non-naturally occurring transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK) or a ubiquitin C (UBC) promoter.
2. The gene therapy construct of numbered embodiment 1, wherein the ZFP comprises a recognition helix region designated ZFP46025 or ZFP45723.
3 The gene therapy construct of numbered embodiment 1 or 2, wherein the ZFP is codon-optimized.
4. The gene therapy construct of any one of the preceding numbered embodiments, wherein the ZFP comprises a nucleotide sequence having at least 60% identity to any one of SEQ ID NO: 10-29.
5. The gene therapy construct of numbered embodiment 3, wherein the ZFP comprises a nucleotide sequence having at least 65%, 70%, 75%, 80%, 85%, 90%, 95% or greater identity to any one of SEQ ID NO: 10-29.
6. The gene therapy construct of numbered embodiment 4, wherein the ZFP-TF comprises a nucleotide sequence having 100% identity to any one SEQ ID NO: 10-29.
7. A gene therapy construct comprising a non-naturally occurring codon optimized transcription factor (ZFP-TF) comprising a zinc-finger protein (ZFP) sequence and a sequence encoding a transcriptional repression domain,
   wherein the ZFP comprises a recognition helix region designated ZFP46025 or ZFP45723, and
   wherein the ZFP binds to a target site in a mutant HTT (mHTT) gene.
8. A gene therapy construct comprising a non-naturally occurring codon-optimized transcription factor (ZFP-TF) comprising a zinc-finger protein (ZFP) sequence and a sequence encoding a transcriptional repression domain, wherein the ZFP-TF comprises a nucleotide sequence having at least 85% identity to any one of SEQ ID NO: 11-22 or SEQ ID NO: 24-29, and wherein the ZFP binds to a target site in a mutant HTT (mHTT) gene.
9. The ZFP-TF of numbered embodiment 7 or 8, comprising a nucleotide sequence having 90%, 95% or greater identity to any one of SEQ ID NO: 11-22 or SEQ ID NO: 24-29.
10. The ZFP-TF of numbered embodiment 9, comprising 100% identity to any one of SEQ ID NO: 11-22 or SEQ ID NO: 24-29.
11. The ZFP-TF of any one of numbered embodiments 7-10, wherein the ZFP-TF expression is driven by a phosphoglycerate kinase 1 (PGK), a ubiquitin C (UBC), an EFS or an EF1alpha promoter.
12. The ZFP-TF of any one of the preceding numbered embodiments, wherein the recognition helix region comprises the amino acid sequence of one of SEQ ID NO: 1-5 or SEQ ID NO: 7-9.
13. The ZFP-TF of any one of the preceding numbered embodiments, wherein the target site comprises a CAG repeats domain of the mHTT gene.
14. The ZFP-TF of numbered embodiment 12, wherein the target site recognizes a sequence comprising 70%, 75%, 80%, 85%, 90%, 95% or greater identity to SEQ ID NO: 6.
15. The ZFP-TF of numbered embodiment 13, wherein the target site recognizes a sequence comprising 100% identity to SEQ ID NO: 6.
16. The ZFP-TF of any one of the preceding numbered embodiments, further comprising a sequence encoding a nuclear localization sequence (NLS).
17. The ZFP-TF of numbered embodiment 16, wherein the NLS is SV40.
18. The ZFP-TF of any one of the preceding numbered embodiments, further comprising inverted terminal repeats (ITRs) flanking the promoter.
19. The ZFP-TF of any one of the preceding numbered embodiments, further comprising a human growth hormone (hGH) poly adenylation signal.
20. The gene therapy construct of any one of the preceding numbered embodiments, wherein the gene therapy construct is delivered using a viral vector.
21. The gene therapy construct of numbered embodiment 20, wherein the viral vector is adeno-associated virus (AAV) or a virus-like particle (VLP).
22. The gene therapy construct of any one of the preceding numbered embodiments, wherein the gene therapy construct is delivered using a lipid nanoparticle (LNP) or liposome.
23. A recombinant rAAV vector comprising a gene therapy construct encoding a non-naturally occurring transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK), a ubiquitin C (UBC), an EFS or an EF1 alpha promoter.
24. An rAAV vector comprising a gene therapy construct comprising a non-naturally occurring codon optimized transcription factor (ZFP-TF) comprising a zinc-finger protein (ZFP) sequence and a sequence encoding a transcriptional repression domain, wherein the ZFP comprises a recognition helix region designated ZFP46025 or ZFP45723, and wherein the ZFP binds to a target site in a mutant HTT (mHTT) gene.
25. An rAAV vector comprising a gene therapy construct comprising a non-naturally occurring codon-optimized transcription factor (ZFP-TF) comprising a zinc-finger protein (ZFP) sequence and a sequence encoding a transcriptional repression domain, wherein the ZFP-TF comprises a nucleotide sequence having at least 85% identity to any one of SEQ ID NO: 11-22 or SEQ ID NO: 24-29, and wherein the ZFP binds to a target site in a mutant HTT (mHTT) gene.
26. The rAAV vector of any one of numbered embodiments 23-25, wherein the rAAV vector is AAV1, AAV2, AAV5, AAV7, AAV9, or AAVrh10.
27. The rAAV vector of any one of numbered embodiments 23-25, wherein the rAAV vector comprises a capsid protein that penetrates a blood brain barrier (BBB).
28. The rAAV vector of numbered embodiment 27, wherein the rAAV vector is VCAP-101, VCAP-102, 9P801, VCAP-100, VCAP-103, PAL1A, PAL1B, PAL1C, PAL2, CereAAV, Dyno bCAP1, AAV.CAP-B10, AAV.CAP-B20, AAV2-BRIN, AAV2-BR1, STAC-BBB®, or AAV-TT, or AAV-BI-hTFR1.
29. A lipid nanoparticle comprising the gene therapy construct of any one of the preceding numbered embodiments.
30. A pharmaceutical composition comprising the rAAV vector or lipid nanoparticle of any one of the preceding numbered embodiments.
31. A method of modulating expression of a mutant Huntington's Disease (mHTT) allele comprising administering the pharmaceutical composition of numbered embodiment 30.
32. A method of modulating expression of a mutant Huntington's Disease HTT (mHTT) allele comprising
   administering an rAAV or a lipid nanoparticle comprising one or more gene therapy constructs encoding a non-naturally occurring transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK), a ubiquitin C (UBC), an EFS, or an EF1alpha promoter, wherein the ZFP binds to a target site in a mutant HTT (mHTT) gene, and wherein upon administration, expression of a mutant (mHTT) allele is reduced.
33. A method of treating Huntington's Disease comprising
   administering to a subject in need thereof, an rAAV or a lipid nanoparticle comprising one or more gene therapy constructs encoding a non-naturally occurring transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK), a ubiquitin C (UBC), an EFS or an EF1alpha promoter, wherein the ZFP binds to a target site in a mutant HTT (mHTT) gene, and wherein upon administration, one or more symptoms associated with Huntington's disease is reduced or relieved.
34. The method of any one of the preceding numbered embodiments, wherein the ZFP comprises a recognition helix region designated ZFP46025 or ZFP45723.
35. The method of any one of the preceding numbered embodiments, wherein the ZFP is codon-optimized.
36. The method of any one of the preceding numbered embodiments, wherein the ZFP-TF comprises a nucleotide sequence having at least 85% identity to any one of SEQ ID NO: 11-22 or SEQ ID NO: 24-29.
37. A method of treating Huntington's disease comprising administering to a subject in need thereof a therapeutically effective amount of a pharmaceutical composition of numbered embodiment 30.
38. The method of numbered embodiment 33, wherein the one or more symptoms is cell death.
39. The method of numbered embodiment 33, wherein the one or more symptoms is apoptosis.
40 The method of numbered embodiment 33, wherein the one or more symptoms is motor deficiency.
41. The method of any one of numbered embodiments 33-40, wherein the administration is intrathecal, intracerebroventricular, intranasal or intravenous.
42. The method of any one of numbered embodiments 33-40, wherein the administration is via focused ultrasound.
43. The method of any one of the preceding numbered embodiments, wherein the administration is to the central nervous system (CNS).
44. The method of any one of the preceding numbered embodiments, wherein the administration to the brain is to one or more of striatum, cortex, caudate, putamen, thalamus, or globus pallidus regions.
45. The method of any one of the preceding numbered embodiments, wherein the administration is systemic.
46. The method of any one of the preceding numbered embodiments, wherein the administration is to nerve cells.
47. A method of treating Huntington's Disease comprising administering to a subject in need thereof, an rAAV or a lipid nanoparticle comprising one or more gene therapy constructs of any one of the preceding claims, wherein the rAAV is a BBB-penetrant rAAV, wherein the administration is intravenous, and wherein upon administration, one or more symptoms associated with Huntington's disease is reduced or relieved.

1. A gene therapy construct comprising a non-naturally occurring codon optimized transcription factor (ZFP-TF) comprising a zinc-finger protein (ZFP) sequence and a sequence encoding a transcriptional repression domain,
wherein the ZFP comprises a recognition helix region designated ZFP46025 or ZFP45723, and
wherein the ZFP binds to a target site in a mutant HTT (mHTT) gene.

2. A gene therapy construct comprising a non-naturally occurring codon-optimized transcription factor (ZFP-TF) comprising a zinc-finger protein (ZFP) sequence and a sequence encoding a transcriptional repression domain, wherein the ZFP-TF comprises a nucleotide sequence having at least 85% identity to any one of SEQ ID NO: 11-22 or SEQ ID NO: 24-29, and wherein the ZFP binds to a target site in a mutant HTT (mHTT) gene.

3. The ZFP-TF of claim 1 or 2, comprising a nucleotide sequence having 90%, 95% or greater identity to any one of SEQ ID NO: 11-22 or SEQ ID NO: 24-29.

4. The ZFP-TF of claim 3, comprising 100% identity to any one of SEQ ID NO: 11-22 or SEQ ID NO: 24-29.

5. The ZFP-TF of any one of claims 1-4, wherein the ZFP-TF expression is driven by a phosphoglycerate kinase 1 (PGK), ubiquitin C (UBC), EFS, or EF1alpha promoter.

6. The ZFP-TF of any one of the preceding claims, wherein the recognition helix region comprises the amino acid sequence of one of SEQ ID NO: 1-5 or SEQ ID NO: 7-9.

7. The ZFP-TF of any one of the preceding claims, wherein the target site comprises a CAG repeats domain of the mHTT gene.

8. The ZFP-TF of claim 6, wherein the target site recognizes a sequence comprising 70%, 75%, 80%, 85%, 90%, 95% or greater identity to SEQ ID NO: 6.

9. The ZFP-TF of claim 7, wherein the target site recognizes a sequence comprising 100% identity to SEQ ID NO: 6.

10. The ZFP-TF of any one of the preceding claims, further comprising a sequence encoding a nuclear localization sequence (NLS).

11. The ZFP-TF of claim 10, wherein the NLS is SV40.

12. The ZFP-TF of any one of the preceding claims, further comprising inverted terminal repeats (ITRs) flanking the promoter.

13. The ZFP-TF of any one of the preceding claims, further comprising a human growth hormone (hGH) poly adenylation signal.

14. The gene therapy construct of any one of the preceding claims, wherein the gene therapy construct is delivered using a viral vector.

15. The gene therapy construct of claim 14, wherein the viral vector is adeno-associated virus (AAV), lentivirus, adenovirus or a virus-like particle (VLP).

16. The gene therapy construct of any one of the preceding claims, wherein the gene therapy construct is delivered using a lipid nanoparticle (LNP) or liposome.

17. A recombinant rAAV vector comprising a gene therapy construct encoding a non-naturally occurring transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK), a ubiquitin C (UBC), an EFS, or an EF1alpha promoter.

18. An rAAV vector comprising a gene therapy construct comprising a non-naturally occurring codon optimized transcription factor (ZFP-TF) comprising a zinc-finger protein (ZFP) sequence and a sequence encoding a transcriptional repression domain, wherein the ZFP comprises a recognition helix region designated ZFP46025 or ZFP45723, and wherein the ZFP binds to a target site in a mutant HTT (mHTT) gene.

19. An rAAV vector comprising a gene therapy construct comprising a non-naturally occurring codon-optimized transcription factor (ZFP-TF) comprising a zinc-finger protein (ZFP) sequence and a sequence encoding a transcriptional repression domain, wherein the ZFP-TF comprises a nucleotide sequence having at least 85% identity to any one of SEQ ID NO: 11-22 or SEQ ID NO: 24-29, and wherein the ZFP binds to a target site in a mutant HTT (mHTT) gene.

20. The rAAV vector of any one of claims 17-19, wherein the rAAV vector is AAV1, AAV2, AAV5, AAV7, AAV9, or AAVrh10.

21. The rAAV vector of any one of claims 17-19, wherein the rAAV vector comprises a capsid protein that penetrates a blood brain barrier (BBB).

22. The rAAV vector of claim 21, wherein the rAAV vector is VCAP-101, VCAP-102, 9P801, VCAP-100, VCAP-103, PAL1A, PAL1B, PAL1C, PAL2, CereAAV, Dyno bCAP1, AAV.CAP-B10, AAV.CAP-B20, AAV2-BRIN, AAV2-BR1, STAC-BBB, or AAV-TT, or AAV-BI-hTFR1.

23. A lipid nanoparticle comprising the gene therapy construct of any one of the preceding claims.

24. A pharmaceutical composition comprising the rAAV vector or lipid nanoparticle of any one of the preceding claims.

25. A method of modulating expression of a mutant Huntington's Disease (mHTT) allele comprising administering the pharmaceutical composition of claim 24.

26. A method of modulating expression of a mutant Huntington's Disease HTT (mHTT) allele comprising administering an rAAV or a lipid nanoparticle comprising one or more gene therapy constructs encoding a non-naturally occurring transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK), a ubiquitin C (UBC), an EFS, or an EF1alpha promoter, wherein the ZFP binds to a target site in a mutant HTT (mHTT) gene, and wherein upon administration, expression of a mutant (mHTT) allele is reduced.

27. A method of treating Huntington's Disease comprising administering to a subject in need thereof, an rAAV or a lipid nanoparticle comprising one or more gene therapy constructs encoding a non-naturally occurring transcription factor (ZFP-TF) comprising a zinc-finger protein (ZFP) sequence and a sequence encoding a transcriptional repression domain, wherein the ZFP-TF expression is driven by a phosphoglycerate kinase 1 (PGK), a ubiquitin C (UBC), an EFS, or an EF1alpha promoter, wherein the ZFP binds to a target site in a mutant HTT (mHTT) gene, and wherein upon administration, one or more symptoms associated with Huntington's disease is reduced or relieved.

28. The method of any one of the preceding claims, wherein the ZFP comprises a recognition helix region designated ZFP46025 or ZFP45723.

29. The method of any one of the preceding claims, wherein the ZFP is codon-optimized.

30. The method of any one of the preceding claims, wherein the ZFP-TF comprises a nucleotide sequence having at least 85% identity to any one of SEQ ID NO: 11-22 or SEQ ID NO: 24-29.

31. A method of treating Huntington's disease comprising administering to a subject in need thereof a therapeutically effective amount of a pharmaceutical composition of claim 24.

32. The method of claim 27, wherein the one or more symptoms is cell death.

33. The method of claim 27, wherein the one or more symptoms is apoptosis.

34. The method of claim 27, wherein the one or more symptoms is motor deficiency.

35. The method of any one of claims 27-34, wherein the administration is intrathecal, intracerebroventricular, intranasal or intravenous.

36. The method of any one of claims 27-34, wherein the administration is via focused ultrasound.

37. The method of any one of the preceding claims, wherein the administration is to the brain.

38. The method of any one of the preceding claims, wherein the administration to the brain is to one or more of striatum, cortex, caudate, putamen, thalamus, and globus pallidus regions.

39. The method of any one of the preceding claims, wherein the administration is systemic.

40. The method of any one of the preceding claims, wherein the administration is to the central nervous system (CNS).

41. A method of treating Huntington's Disease comprising administering to a subject in need thereof, an rAAV or a lipid nanoparticle comprising one or more gene therapy constructs of any one of the preceding claims, wherein the rAAV is a BBB-penetrant rAAV, wherein the administration is intravenous, and wherein upon administration, one or more symptoms associated with Huntington's disease is reduced or relieved.
